(19) United States
(12) Patent Application Publication
(10) Pub. No.: US 2006/0094693 A1
(43) Pub. Date: May 4, 2006

(54) Methods and compositions for treating glucose-associated conditions, metabolic syndrome, dyslipidemias and other conditions

(75) Inventors: Nazneen Aziz, Lexington, MA (US); Michael I. Jesson, Hopedale, MA (US); Paul A. McLean, Cambridge, MA (US); Glenn T. Miller, Merrimac, MA (US); Barry Jones, Cambridge, MA (US)

Correspondence Address: WOLF GREENFIELD & SACKS, PC;FEDERAL RESERVE PLAZA, 600 ATLANTIC AVENUE, BOSTON, MA, 02210-2206, US

(73) Assignee: Point Therapeutics, Inc., Boston, MA (US)

(21) Appl. No.: 11/233,532

(22) Filed: Sep. 21, 2005

Related U.S. Application Data
(60) Provisional application No. 60/704,157, filed on Jul. 29, 2005.
(60) Provisional application No. 60/700,871, filed on Jul. 19, 2005.
(60) Provisional application No. 60/622,466, filed on Oct. 27, 2004.
(60) Provisional application No. 60/612,069, filed on Sep. 21, 2004.

(51) Int. Cl. A61K 31/69

(52) U.S. Cl. 514/64

(57) ABSTRACT

The invention relates, in part, to of Glu-boroPro containing compounds and methods of use thereof in the prevention or management of conditions that are associated with impaired glucose tolerance such as diabetes. The invention also relates to compositions of Glu-boroPro containing compounds and methods of use thereof in the prevention or management of conditions such as metabolic syndrome, dyslipidemias, inflammation, cardiovascular disorders such as hypertension and atherosclerosis, and to reduce body weight or prevent weight gain. The compounds of the invention are also useful in lowering levels of triglycerides, free fatty acids, C-reactive protein (CRP), HbA₁C, total glycosylated hemoglobin (TGHb), in increasing insulin sensitivity index and in stimulating insulin release.

### RELATED APPLICATIONS

This application claims priority under 35 U.S.C. § 119(e) from U.S. provisional application Ser. No. 60/704,157, filed Jul. 29, 2005, and from U.S. provisional application Ser. No. 60/700,871, filed Jul. 19, 2005, and from U.S. provisional application Ser. No. 60/622,466, filed Oct. 27, 2004, and from U.S. provisional application Ser. No. 60/612,069, filed Sep. 21, 2004.

### FIELD OF THE INVENTION

The invention relates, in part, to compositions of glutamic acid boroproline containing compounds and uses thereof for the treatment and prevention of conditions that are associated with impaired glucose tolerance, such as type 2 diabetes and other conditions such as metabolic syndrome, dyslipidemias, cardiovascular disorders, inflammation and obesity.

### BACKGROUND OF THE INVENTION

Type 2 diabetes accounts for 90-95 per cent of all diabetes and results from insulin resistance in muscle and impaired function of the pancreatic beta (β)-cells that produce insulin in response to dietary sugar (1). In advanced stages of the disease, β-cell function can degenerate to a point where insulin therapy is required.

One potential approach to treatment is to enhance the incretin effect whereby insulin secretion in response to orally ingested glucose is amplified by small peptide hormones. Two gut-derived hormones, glucagon-like peptide-1 (GLP-1) and gastric inhibitory protein (GIP) act through cognate G-protein-coupled receptors on β-cells to potentiate the stimulation of insulin secretion in response to dietary glucose (3).

The incretin effect of both hormones is limited in vivo, however, because they are rapidly inactivated by the serine protease DPP-IV. DPP-IV is a ubiquitously expressed serine protease that can cleave dipeptides from the N-termini of polypeptides in which proline or alanine occupies the penultimate position at the N-terminus (5). A soluble form of DPP-IV is present in blood, and the enzyme is expressed as a 220 kDa type-II integral-membrane protein on the surface of various cell types, including epithelial, endothelial and lymphoid cells (6).

Adequate control of hyperglycemia in patients with type 2 diabetes can attenuate the development of complications such as retinopathy and nephropathy and cardiovascular complications (2). Ideally, the goal of treatment should be to intervene when impaired glucose tolerance is initially detected.

### SUMMARY OF THE INVENTION

The invention relates in part to the use of glutamic acid boroproline (Glu-boroPro) containing compounds in the treatment (and prevention) of glucose-associated conditions such as type 2 diabetes.

The invention also relates to the use of Glu-boroPro containing compounds in the treatment (and prevention) of conditions that are not necessarily glucose-associated such as metabolic syndrome, dyslipidemias, and cardiovascular disorders. The compounds can also be used to prevent weight gain or to reduce body weight in subjects who may or may not be obese.

The compounds of the invention are also useful in lowering the levels of triglycerides, free fatty acids, C-reactive protein (CRP), HbA₁C, and/or total glycosylated hemoglobin (TGHb) in a subject. The compounds of the invention are also useful in increasing the insulin sensitivity index in a subject, stimulating insulin release from the pancreas, and reducing food intake by a subject.

The invention is premised in part on the finding that Glu-boroPro, is far superior to other compounds including other boroproline containing compounds in the treatment and prevention of such conditions. This is surprising because of the structural similarity of the compounds tested and their relative equivalence in other assays (e.g. in vivo DPP-IV inhibition). Glu-boroPro does not appreciably induce cytokine and/or chemokine production and does not stimulate the immune response. The ability of Glu-boroPro to inhibit DPP-IV without inducing cytokine and/or chemokine production and/or without stimulating the immune response are desirable features in treating the conditions encompassed by the invention.

The invention embraces Glu-boroPro containing compounds and these include Glu-boroPro or compounds that are converted to Glu-boroPro (e.g., prodrugs of Glu-bororPro which when acted upon in vivo release Glu-boroPro). For convenience and brevity, the specification refers to Glu-boroPro containing compounds, however, it is to be understood that the invention intends to embrace compounds in which the boronic acid reactive group is replaced with a different reactive group (as described in greater detail herein) such as but not limited to fluoralkylketones, alphaketo amides, alphaketo esters, alphaketo acids, phosphonates, cyanopyrrolidines and thiazolides.

The invention thus provides, interalia, compositions comprising Glu-boroPro containing compounds and methods of use thereof for treating and preventing glucose-associated conditions. These conditions include but are not limited to type 1 diabetes (insulin dependent diabetes mellitus or IDDM), type 2 diabetes (non-insulin dependent diabetes mellitus or NIDDM), gestational diabetes, diabetic ketoacidosis (DKA), insulin resistance, impaired glucose tolerance, some forms of obesity, hyperglycemia (elevated blood glucose concentration), hyperinsulinemia, hyperlipidemia, hyperlipoproteinemia, and various metabolic disorders. The invention also intends to embrace treatment of conditions which would benefit from pancreatic β cell preservation, reduced glucagon levels, or increased insulin availability.

Thus, in one aspect, the invention provides a method for treating a subject having or at risk of developing a glucose-associated condition (such as type 2 diabetes) comprising administering to a subject in need thereof an agent having a structure 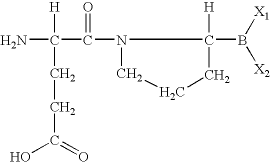
or a prodrug thereof in an effective amount to treat the subject, wherein each X₁ and X₂ is, independently, a hydroxyl group or a group capable of being hydrolyzed to a hydroxyl group in aqueous solution at physiological pH.

In one embodiment the subject is obese or has impaired oral glucose tolerance. The agent may be administered orally, although other routes of administration include, for example, subcutaneous and intravenous administration.

In one embodiment the agent is administered within 30 minutes of a meal, while in other embodiments the agent is administered at a time that is independent of food or beverage intake.

The method may further comprise administering a second agent to the subject. The nature of the second agent will depend on which of the glucose-associated conditions the subject has or is at risk of developing. In one embodiment the second agent is a second anti-diabetic agent. The agent and the second anti-diabetic agent may be administered in an alternating manner.

In yet another aspect, the invention provides a method for reducing blood glucose comprising orally administering to a subject in need thereof prior to a glucose challenge Glu-boroPro having a structure 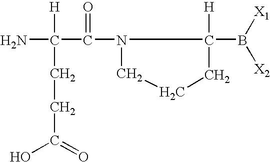
or a prodrug thereof in an effective amount to reduce the blood glucose level wherein each X₁ and X₂ is, independently, a hydroxyl group or a group capable of being hydrolyzed to a hydroxyl group in aqueous solution at physiological pH.

In one embodiment Glu-boroPro or the prodrug is administered 15 minutes prior to a glucose challenge. In one embodiment the glucose challenge is food or beverage intake. In another embodiment the blood glucose level is reduced for an extended period of time such as, but not limited to, 6 hours, 12 hours, 24 hours, 36 hours or 48 hours. In one embodiment the subject has or is at risk of developing type 2 diabetes. In another embodiment the subject is obese or has impaired oral glucose tolerance.

In one embodiment the effective amount is an amount that reduces blood glucose at least 40% relative to an untreated subject.

In yet another aspect, the invention provides a method for treating a subject having type 2 diabetes comprising orally administering to a subject in need thereof 15 minutes prior to glucose challenge, an agent having a structure of 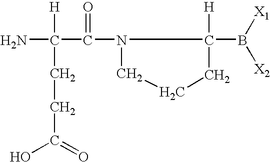
wherein each X₁ and X₂ is a hydroxyl group, in an amount effective to reduce blood glucose level, after glucose challenge, by at least 40% relative to an untreated subject (i.e., an untreated subject having type 2 diabetes).

According to another aspect of the invention, a composition is provided that comprises an agent having a structure 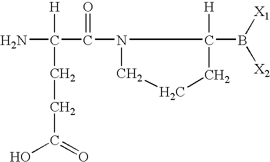
or a prodrug thereof and a second agent, such as, but not limited to, an anti-diabetic agent wherein each X₁ and X₂ is, independently, a hydroxyl group or a group capable of being hydrolyzed to a hydroxyl group in aqueous solution at physiological pH.

In one embodiment the composition is a pharmaceutical preparation and it comprises a pharmaceutically-acceptable carrier. In another embodiment the agent is present in a unit dosage of between 750 μg to 9000 μg. In yet another embodiment the unit dosage is an amount less than that required to stimulate cytokine or chemokine induction.

In yet another aspect, the invention provides a pharmaceutical preparation comprising an agent having a structure 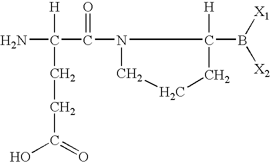
or a prodrug thereof in a pharmaceutically-acceptable carrier and in a unit dosage that is effective for reducing blood glucose wherein each X₁ and X₂ is, independently, a hydroxyl group or a group capable of being hydrolyzed to a hydroxyl group in aqueous solution at physiological pH.

In one embodiment the unit dosage is a one a day unit dosage. In a related embodiment the one a day unit dosage is 750 to 9,000 μg per day. In another embodiment the unit dosage is an amount that reduces blood glucose by at least 40% as compared to an untreated subject. In another embodiment the unit dosage is an amount that reduces blood glucose to a level that is +/−10% of blood glucose level in a non-diabetic subject.

In yet another aspect, the invention provides a kit comprising any of the foregoing compositions and agents formulated for oral administration and a daily dispenser. In one embodiment the composition or agent is formulated as a tablet, pill, capsule or caplet.

In another embodiment the kit contains a one month supply of the composition. In another embodiment the daily dispenser is a blister-pack dispenser or a dial dispenser.

According to another aspect of the invention, a method for treating a subject having or at risk of developing a dyslipidemia is provided. The method comprises administering to a subject in need thereof an agent having a structure 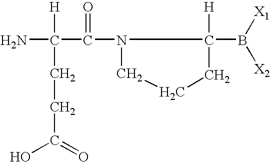
or a prodrug thereof in an effective amount to treat the subject wherein each X₁ and X₂ is, independently, a hydroxyl group or a group capable of being hydrolyzed to a hydroxyl group in aqueous solution at physiological pH.

In some embodiments the subject has Familial Hypertriglyceridemia, Familial Apoprotein CII deficiency, Hepatic Lipase Deficiency, Familial Combined Hyperlipidemia, Dysbetalipoproteinemia, or Familial Lipoprotein Lipase Deficiency.

In some embodiments the subject has Hyperlipidemia Type I, II, III, IV or V (also called Hyperlipoproteinemia Type I, II, III, IV, and V respectively). In some important embodiments the subject has Hyperlipidemia Type IV or V. In some embodiments the subject is at risk for pancreatitis. The subject may or may not respond to diet. In some embodiments the subject is being treated with fibric acid derivative(s) (fibrates). In other embodiments the subject is not and has not been treated with fibric acid derivative(s).

In some embodiments the dyslipidemia comprises an elevated triglyceride level. In some preferred embodiments the elevated triglyceride level is above about 150 mg/dl. In some embodiments the dyslipidemia comprises an elevated total cholesterol level. In some preferred embodiments the elevated total cholesterol level is above about 200 mg/dl. In some embodiments the dyslipidemia comprises an elevated level of low density lipoprotein (LDL). In some preferred embodiments the elevated LDL level is above about 130 mg/dl. In some embodiments the dyslipidemia comprises an elevated low density lipoprotein (LDL) to high density lipoprotein (HDL) ratio. In some preferred embodiments the elevated LDL to HDL ratio is above about 1. In some embodiments the dyslipidemia comprises an elevated total cholesterol to HDL ratio. In some preferred embodiments the elevated total cholesterol to HDL ratio is above about 2. In some embodiments the dyslipidemia comprises an elevated apolipoprotein B (apo-B) level. In other embodiments the dyslipidemia comprises an elevated Lp(a) level. In some embodiments the dyslipidemia comprises a reduced level of high density lipoprotein (HDL). In some preferred embodiments the reduced HDL level is below about 40 mg/dl. In some embodiments the dyslipidemia comprises a reduced apolipoprotein A1 (apo A-1) level.

In some embodiments the method further comprises administering a second agent to the subject. In some preferred embodiments the second agent is an anti-hyperlipidemia agent.

In some embodiments the subject may be obese.

According to another aspect of the invention, a method for treating a subject having or at risk of developing metabolic syndrome is provided. The method comprises administering to a subject in need thereof an agent having a structure 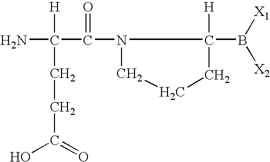
or a prodrug thereof in an effective amount to treat the subject wherein each X₁ and X₂ is, independently, a hydroxyl group or a group capable of being hydrolyzed to a hydroxyl group in aqueous solution at physiological pH.

In some embodiments the subject is not diabetic. In other embodiments the subject does not have glucose intolerance. In some embodiments the subject has a fasting blood sugar below about 120 mg/dl. In other embodiments the subject does not have elevated blood pressure. In some embodiments the subject is not dyslipidemic. In other embodiments the subject is not obese.

The method may comprise administering a second agent to the subject. The second agent may be an anti-diabetic agent, examples of which are provided herein.

The second agent may be an anti-hyperlipidemia agent. The agent and the anti-hyperlipidemia agent may be administered in an alternating manner.

The second agent may be an anti-hypertension agent. The agent and the anti-hypertension agent may be administered in an alternating manner.

In yet another aspect of the invention, a method for treating a subject having or at risk of developing a cardiovascular disorder is provided. The method comprises administering to a subject in need thereof an agent having a structure 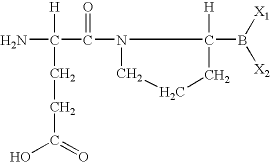
or a prodrug thereof in an effective amount to treat the subject wherein each X₁ and X₂ is, independently, a hydroxyl group or a group capable of being hydrolyzed to a hydroxyl group in aqueous solution at physiological pH.

In some embodiments the cardiovascular disorder comprises an elevated blood pressure (hypertension). The elevated blood pressure may be an elevated systolic blood pressure, an elevated diastolic pressure, or a combination of an elevated systolic and diastolic pressure. In some embodiments the cardiovascular disorder comprises pre-hypertension.

In some embodiments the systolic blood pressure is above about 120 mm Hg. In some embodiments the diastolic blood pressure is above about 80 mm Hg.

The method may comprise administering a second agent to the subject. The second agent may be an anti-hypertension agent.

In some embodiments the cardiovascular disorder comprises atherosclerosis.

In some embodiments the subject is obese. In other embodiments the subject is not obese.

According to still another aspect of the invention, a method for controlling body weight of a subject is provided. The method comprises administering to a subject in need thereof an agent having a structure 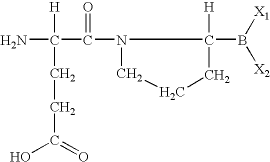
or a prodrug thereof in an effective amount to control the body weight of the subject wherein each X₁ and X₂ is, independently, a hydroxyl group or a group capable of being hydrolyzed to a hydroxyl group in aqueous solution at physiological pH.

In some embodiments controlling body weight of a subject comprises reducing the body weight of the subject. In other embodiments, controlling body weight comprises preventing body weight gain in the subject.

The subject may or may not be obese. In some embodiments the subject is not diabetic. In other embodiments the subject does not have glucose intolerance. In some embodiments the subject does not have elevated blood pressure. In other embodiments the subject is not dyslipidemic.

The method may comprise administering a second agent to the subject. The second agent may be an anti-diabetic agent, an anti-hyperlipidemia agent, an anti-hypertension agent, or an anti-obesity agent.

In yet another aspect of the invention, a method for increasing the insulin sensitivity index in a subject is provided. The method comprises administering to a subject in need thereof an agent having a structure 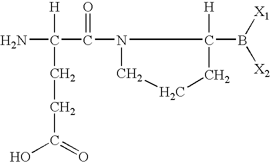
or a prodrug thereof in an effective amount to increase the insulin sensitivity in the subject wherein each X₁ and X₂ is, independently, a hydroxyl group or a group capable of being hydrolyzed to a hydroxyl group in aqueous solution at physiological pH.

In some embodiments the subject has a glucose-associated condition. The glucose-associated condition may be glucose intolerance or diabetes. In some embodiments the subject has metabolic syndrome and may or may not be diabetic.

The method may further comprise administering a second agent to the subject. The second agent may be an anti-diabetic agent, anti-hyperglycemia agent, an anti-hypertension agent, or an anti-hyperlipidemia agent. The agent and the second agent may be administered in an alternating manner.

In some embodiments the subject is obese. In other embodiments the subject is not obese.

According to still another aspect of the invention, a method for lowering free fatty acid levels in a subject is provided. The method comprises administering to a subject in need thereof an agent having a structure 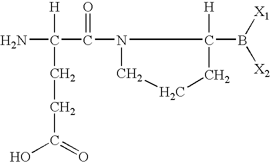
or a prodrug thereof in an effective amount to lower free fatty acid levels in the subject wherein each X₁ and X₂ is, independently, a hydroxyl group or a group capable of being hydrolyzed to a hydroxyl group in aqueous solution at physiological pH.

In some embodiments the subject has a glucose-associated condition. The glucose-associated condition may be glucose intolerance or diabetes. In some embodiments the subject is not diabetic. In some embodiments the subject has metabolic syndrome. The subject may or may not be diabetic. The subject may or may not be obese.

The method may further comprise administering a second agent to the subject. The second agent may be an anti-diabetic agent, an anti-hyperglycemia agent, an anti-hypertension agent, or an anti-hyperlipidemia agent. The agent and the second agent may be administered in an alternating manner.

According to yet another aspect of the invention, a method for lowering HbA₁C in a subject is provided. The method comprises administering to a subject in need thereof an agent having a structure 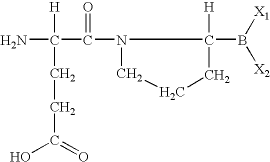
or a prodrug thereof in an effective amount to lower the HbA₁C in the subject wherein each X₁ and X₂ is, independently, a hydroxyl group or a group capable of being hydrolyzed to a hydroxyl group in aqueous solution at physiological pH.

In some embodiments the subject has a glucose-associated condition. The glucose-associated condition may be glucose intolerance or diabetes.

In some embodiments the subject has metabolic syndrome. The subject may or may not be obese.

The method may further comprise administering a second agent to the subject. The second agent may be an anti-diabetic agent, an anti-hyperglycemia agent, an anti-hypertension agent, or an anti-hyperlipidemia agent. The agent and the second agent may be administered in an alternating manner.

In some important embodiments the HbA₁C level is lowered by least about 1% of the total pre-treatment HbA₁C level.

According to still another aspect of the invention, a method for lowering an elevated C-reactive protein (CRP) level in a subject is provided. The method comprises administering to a subject in need thereof an agent having a structure 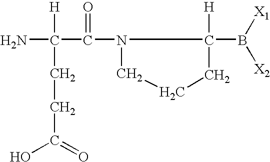
or a prodrug thereof in an effective amount to lower the CRP level in the subject wherein each X₁ and X₂ is, independently, a hydroxyl group or a group capable of being hydrolyzed to a hydroxyl group in aqueous solution at physiological pH.

In some preferred embodiments the elevated CRP level is above about 1 mg/L. In other preferred embodiments the elevated CRP level is above about 1.5 mg/L. In still other preferred embodiments the elevated CRP level is above about 2 mg/L.

The method may further comprise administering a second agent to the subject. The second agent may be an anti-hyperlipidemia agent. The agent and the second agent may be administered in an alternating manner.

In some preferred embodiments the agent is administered independent of food intake.

According to yet another aspect of the invention, a method for treating a subject having or at risk of developing inflammation or an inflammatory disorder is provided. The method comprises administering to a subject in need thereof an agent having a structure: 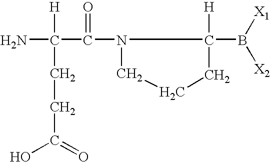
or a prodrug thereof in an effective amount to treat the subject wherein each X₁ and X₂ is, independently, a hydroxyl group or a group capable of being hydrolyzed to a hydroxyl group in aqueous solution at physiological pH.

In some embodiments the subject is not diabetic. In other embodiments does not have glucose intolerance. In some other embodiments the subject has a fasting blood sugar below about 120 mg/dl.

Inflammatory disorders encompassed by the invention are those affecting various organs and tissues. Examples of tissues and organs affected by inflammatory disorders include, for example, the blood vessels, the heart, the joints, the skin, the lung, the eye, the gastrointestinal tract, the kidney, the thyroid, the adrenal, the pancreas, the liver, and the muscles. Examples of inflammatory disorders encompassed by the invention include, but are not limited to, arthritis, rheumatoid arthritis, asthma, inflammatory bowel diseases (e.g., Crohn's disease and ulcerative colitis), chronic obstructive pulmonary disease (COPD), allergic rhinitis, vasculitis (e.g. polyarteritis nodosa, temporal arteritis, Behcet syndrome), psoriasis, systemic lupus erythematosis (SLE), chronic thyroiditis, Hashimoto's thyroiditis, and Addison s disease.

The method may further comprise administering a second agent to the subject. The second agent may be an anti-inflammatory agent. Examples of anti-inflammatory agents that may be used are listed below. The agent and the anti-inflammatory agent may be administered in an alternating manner.

In another aspect of the invention, a method of treating a subject having metabolic syndrome is provided. The method comprises administering to a subject in need thereof a compound(s) (or agent(s), as used interchangeably herein) having a structure:
PR (Formula I)
wherein P is a targeting group which binds to the reactive site of post proline-cleaving enzyme, and wherein R is a reactive group capable of reacting with a functional group in a post proline cleaving enzyme, preferably in the reactive site of the post proline cleaving enzyme. P may be a peptide or a peptidomimetic. The reactive compound may be selected from the group consisting of organo boronates, organo phosphonates, fluoroalkylketones, alphaketos, N-peptiolyl-O-acylhydroxylamines, azapeptides, azetidines, fluoroolefins dipeptide isoesteres, peptidyl (alpha-aminoalkyl) phosphonate esters, aminoacyl pyrrolidine-2-nitriles and 4-cyanothiazolidides. In some important embodiments the compounds of the invention are Glu-boro-proline containing compounds.

Formula I compounds include compounds having a structure: (Formula II) 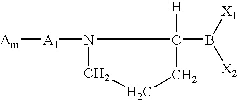
wherein m is an integer between 0 and 10, inclusive; A and A₁ may be a naturally or non-naturally occurring amino acid or a peptide or peptidomemetic such that when A is an amino acid residue each A in Aₘ (i.e., where m>1) may be a different amino acid residue from every other A in Aₘ and when A is a peptide or peptidomimetic m is 1; the C bonded to B is preferably in the R-configuration; and each X₁ and X₂ is, independently, a hydroxyl group or a group capable of being hydrolyzed to a hydroxyl group in aqueous solution at physiological pH.

In some embodiments A and A₁ are independently valine, proline or alanine residues.

In some embodiments Al is alanine, valine, leucine, isoleucine, phenylalanine, tyrosine, tryptophan, serine, threonine, aspartate, glutamate, asparagine, glutamine, lysine, arginine, histidine, cysteine, methionine, or proline.

Thus, in some embodiments the agent is L-Ala-L-boroPro, L-Asp-L-boroPro, L-Glu-L-boroPro, L-Asn-L-boroPro, L-Gln-L-boroPro, L-Lys-L-boroPro, L-Arg-L-boroPro, L-His-L-boroPro, L-Pro-L-boroPro, L-Thr-L-boroPro, L-Ser-L-boroPro, L-Cys-L-boroPro, L-Gly-L-boroPro, L-Tyr-L-boroPro, L-Trp-L-boroPro, L-Phe-L-boroPro, L-Leu-L-boroPro, L-Ile-L-boroPro, L-Met-L-boroPro, or L-Val-L-boroPro. In some embodiments the agent is L-Ile-L-boroPro, L-Met-L-boroPro, or L-Val-L-boroPro. In some preferred embodiments the agent is L-Val-L-boroPro.

In some embodiments m is 0. In some embodiments X₁ and X₂ are hydroxyl groups.

In addition to agents of Formula II, other agents useful in the invention include those in which the proline residue in Formula II is replaced with another amino acid residue such as, for example, lysine, alanine or glycine. As well, derivatives of Formula II in which the boronate group is replaced with a reactive group as described above are also useful in the invention.

Formula I compounds also include compounds having a structure: 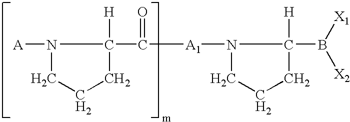
wherein m is an integer between 0 and 10, inclusive; A and A₁ are naturally or non-naturally occurring amino acids or peptides or peptidomimetics; wherein if A is an amino acid residue, it can be a different amino acid residue in each repeating bracketed unit; the C bonded to B is in the R-configuration; and each X₁ and X₂ is, independently, a hydroxyl group or a group capable of being hydrolyzed to a hydroxyl group in aqueous solution at physiological pH.

According to another aspect of the invention, a method for stimulating insulin release from a pancreatic cell is provided. The method comprises contacting a pancreatic cell with a Glu-boroPro containing compound in an effective amount to stimulate insulin release from the pancreatic cell and increase insulin level.

The contacting may be in vivo or ex vivo. When the contacting is in vivo, the agent is administered to the subject.

In some important embodiments, the insulin release is glucose-stimulated. In some preferred embodiments, the Glu-boroPro has the structure 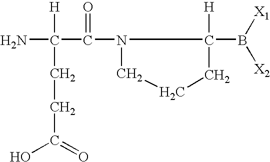
wherein each X₁ and X₂ is, independently, a hydroxyl group or a group capable of being hydrolyzed to a hydroxyl group in a solution at physiological pH.

In some important embodiments, the Glu-boroPro containing compound is cyclic Glu-boroPro.

In some important embodiments, the Glu-boroPro containing compound increases insulin levels in the subject within about 1, 2, 4, 6, 8, 10 12, 14, 16, 18, 20, 25, 30, 35, 40, 45, 50, 55, or 60 minutes following the administration of the Glu-boroPro containing compound. In some embodiments, the Glu-boroPro containing compound lowers the blood glucose level in the subject within about 5, 10, 15, 20, 25, 30, 35, 40, 45, 50, 55, or 60 minutes following administration of the Glu-boroPro containing compound.

The Glu-boroPro containing compound may be administered orally or parenterally. In some important embodiments the agent is administered intravenously. In other important embodiments the agent is administered subcutaneously.

In some important embodiments the subject has or is at risk of developing diabetes. In some preferred embodiments, the diabetes is type 2 diabetes. In some embodiments the subject has diabetic ketoacidosis. In some important embodiments the subject is obese and/or has impaired oral glucose tolerance.

According to still another aspect of the invention a method for increasing the level of adiponectin in a subject is provided. The method comprises administering to a subject in need thereof an agent having a structure 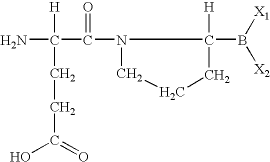
or a prodrug thereof in an effective amount to increase the adiponectin level in the subject wherein each X₁ and X₂ is, independently, a hydroxyl group or a group capable of being hydrolyzed to a hydroxyl group in aqueous solution at physiological pH.

In some important embodiments the plasma adiponectin level is below about 10 μg/mL. In other important embodiments the plasma adiponectin level is below about 5 μg/mL.

The method may further comprise administering a second agent to the subject. The second agent may be an anti-diabetic agent and/or an anti-inflammatory agent. In some important embodiments, the anti-diabetic agent may be a thiazolidinedione.

In some important embodiments the subject is obese.

The following embodiments apply equally to the various aspects of the invention set forth herein unless indicated otherwise.

The agent may comprise a glutamic acid residue bonded to a pyrrolidine ring in an S-configuration.

In some embodiments the agent comprises the carbon of the pyrrolidine ring bonded to the boron in the R-configuration.

The agent may further comprise a mixture of R- and S-enantiomers of boron substituted pyrrolidine. In a related embodiment the mixture of R- and S-enantiomers of boron substituted pyrrolidine contains at least 95%, at least 96%, at least 97%, at least 98%, or at least 99% of the R-enantiomer of boron substituted pyrrolidine.

In some important embodiments the agent is 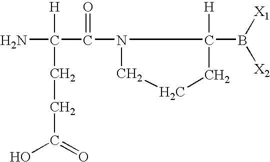
wherein each X₁ and X₂ is, independently, a hydroxyl group or a group capable of being hydrolyzed to a hydroxyl group in aqueous solution at physiological pH.

In some embodiments the agent is a prodrug of Glu-boroPro. Alternatively, the agent may be a cyclic version of Glu-boroPro, an ester of Glu-boroPro, a boroxine derivative of Glu-boroPro, or an alcohol precursor of Glu-boroPro.

In some embodiments the agent has a structure 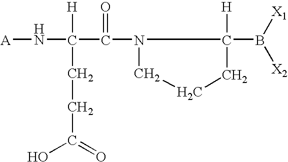
wherein A is a peptide or a peptidomimetic; each X₁ and X₂ is, independently, a hydroxyl group or a group capable of being hydrolyzed to a hydroxyl group in aqueous solution at physiological pH, optionally wherein the C bonded to the B is in the R configuration, and further optionally wherein the bond between the glutamic acid residue and pyrrolidine (i.e., C to N) is in the S configuration.

The agent may have a structure 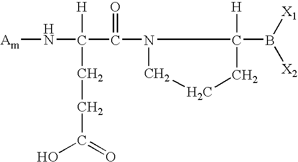
wherein A is any naturally or non-naturally occurring amino acid bonded in either an S- or an R-configuration; m is an integer from 0-100 or more, such that when m is greater than one, each A in Aₘ may be a different amino acid residue from every other A in Aₘ; each X₁ and X₂ is, independently, a hydroxyl group or a group capable of being hydrolyzed to a hydroxyl group in aqueous solution at physiological pH, optionally wherein the C bonded to the B is in the R configuration, and further optionally wherein the bond between the glutamic acid residue and pyrrolidine is in the S configuration.

In some embodiments the agent has a structure 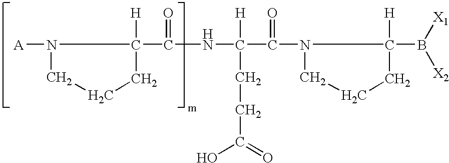
wherein A is any naturally or non-naturally occurring amino acid bonded in an S- or an R-configuration; m is an integer from 0-100 or more, provided that when m is greater than one, A in each repeating bracketed unit can be a different amino acid residue; each X₁ and X₂ is, independently, a hydroxyl group or a group capable of being hydrolyzed to a hydroxyl group in aqueous solution at physiological pH, optionally wherein the C bonded to the B is in the R configuration, and further optionally wherein the bond between the glutamic acid residue and pyrrolidine is in the S configuration.

The agent may be administered at fixed intervals, such as but not limited to every 12 hours, every 24 hours, every 36 hours or every 48 hours. In some preferred embodiments the agent is administered once a day.

The agent may be administered in an effective amount that is less than 5 mg/kg/day, less than 1 mg/kg/day, less than 500 μg/kg/day, less than 250 μg/kg/day, less than 100 μg/kg/day, less than 50 μg/kg/day, less than 25 μg/kg/day or less than 10 μg/kg/day. Alternatively, it may be in the range of 1 μg/kg/day to 200 μg/kg/day.

In some important embodiments the agent is administered orally. In some embodiments the agent is administered parenterally. In some important embodiments the agent is administered intravenously. In other important embodiments the agent is administered subcutaneously.

In some important embodiments the effective amount is an amount that does not stimulate cytokine or chemokine induction by the active agent.

The second anti-diabetic agent may be an insulin, peroxisome proliferator-activated receptor-gamma (PPAR-gamma (y)) agonist, an inhibitor of hepatic glucose production, a stimulator of insulin release from pancreas, a glucosidase inhibitor, an incretin or incretin analogue.

In some embodiments the second anti-diabetic agent is an insulin. The insulin may be a rapid-acting insulin, an intermediate-acting insulin or a long-acting insulin. The rapid-acting insulin may be HUMALOG(®, HUMALOG®& Mix 75/25-Pen, HUMULIN® R, HUMULIN® 50/50, HUMULIN® 70/30, NOVOLIN® R, NOVOLIN® 70/30, NOVOLIN® 70/30 PenFill, NOVOLIN® Innolet, NOVOLOG Mix 70/30, VELOSULIN®, VELOSULIN® BR, ILETIN® I or ILETIN® II. The intermediate-acting insulin may be LENTE® ILETIN® I, LENTE® ILETIN® II, HUMULIN® L, HUMULIN® N, HUMULIN® N pen, NOVOLIN® L, NOVOLIN® N, NOVOLIN® N PenFill, NPH ILETIN® I, NPH ILETIN ® II or NPH-N. The long-acting insulin may be ULTRALENTE®, HUMULIN® U, or Lantus Injection.

In another embodiment the second anti-diabetic agent is a PPARγ agonist. The PPAR-gamma (PPAR-γ) agonist may be a thiazolidinedione such as but not limited to Avandamet (combination of rosiglitazone and metformin), rosiglitazone (Avandia), pioglitazone (Actos), troglitazone (Rezulin), (S)-((3,4-dihydro-2-(phenyl-methyl)-2H-1-benzopyran-6-yl)methyl-thiazolid-ine-2,4-dione (englitazone), 5-{[4-(3-(5-methyl-2-phenyl-4-oxazolyl)-1-oxo- propyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189),5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,-4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-y-1)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione,5-{[4-(2-(methyl-2-pyridinyl-amino)-ethoxy)phenyl]methyl}-thiazolidine-2,-4-dione (rosiglitazone), 5-{[4-(2-(5-ethyl-2-pyridyl)ethoxy)phenyl]-methyl-}thiazolidine-2,4-dione (pioglitazone), 5-{[4-((3,4-dihydro-6-hydroxy-2,5,- 7,8-tetramethyl-2H-1-benzopyran-2-yl)methoxy)-phenyl]-methyl}-thiazolidine-2,4-dione (troglitazone), 5-[6-(2-fluoro-benzyloxy)-naphthalen-2-ylmethyl-]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297). The PPAR-gamma agonist may also be a natural prostaglandin D(2) (PGD(2)) metabolite, 15-deoxy-Delta(12, 14)-prostaglandin J(2) (15d-PGJ(2)).

In another embodiment the second anti-diabetic agent is an inhibitor of hepatic glucose production. The inhibitor of hepatic glucose production may be a biguanide such as but not limited to metformin (GLUCOPHAGE), Avandamet, Glucovance, or Metaglip.

In yet another embodiment the second anti-diabetic agent is a stimulator of insulin release from pancreas such as but not limited to a sulfonylurea or a meglitinide. The sulfonylurea may be acetohexamide (DYMELOR), chlorpropamide (DIABINESE), tolbutamide (ORINASE, RASTINON), glipizide (GLUCOTROL, GLUCOTROL XL), glyburide (DIABETA; MICRONASE; GLYNASE), glimepiride (AMARYL), glisoxepid (PRO-DIABAN), glibenclamide (AZUGLUCON), glibomuride (GLUBORID), tolazamide, carbutamide, gliquidone (GLURENORM), glyhexamide, phenbutamide, tolcyclamide or gliclazide (DIAMICRON). The meglitinide may be Repaglinide (PRANDIN) or nateglinide (STARLIX).

In a further embodiment the second anti-diabetic agent is a glucosidase inhibitor such as but not limited to acarbose (PRECOSE, GLUCOBAY), miglitol (GLYSET, DIASTABOL) or voglibose.

In yet another embodiment the second anti-diabetic agent is an incretin or incretin analogue. The incretin or incretin analogue may be GLP-1, GIP, EXENATIDE (BYETTA) or EXENATIDE LAR.

In still another embodiment the second anti-diabetic agent is a DPP-IV inhibitor selected from the group consisting of alanyl pyrrolidine, isoleucyl thiazolidine, and O-benzoyl hydroxylamine.

These and other aspects of the invention, as well as various advantages and utilities, will be more apparent with reference to the detailed description of the invention. Each aspect of the invention can encompass various embodiments as will be understood.

### BRIEF DESCRIPTION OF THE DRAWINGS

FIG. 1A is a graph showing the level of DPP-IV activity in vitro as a function of concentration of the indicated amino acid boroPro compounds.

FIG. 1B is a graph showing the level of DPP-IV activity in vitro as a function of time after exposure of DPP-IV to the indicated amino acid boroPro compounds.

FIG. 2A is a graph showing the level of DPP-IV activity in vivo as a function of dose of Glu-boroPro.

FIG. 2B is a graph showing the level of DPP-IV activity in vivo as a function of time after exposure to Glu-boroPro.

FIG. 3 is a graph showing the level of G-CSF produced following in vitro exposure of human bone marrow stromal cells to the indicated amino acid boroPro compounds.

FIG. 4A is a graph showing the level of serum DPP-IV activity in vivo at 2 hours after administration of the indicated doses of various amino acid boroPro compounds.

FIG. 4B is a graph showing the level of serum KC in vivo at 2 hours after administration of the indicated doses of various amino acid boroPro compounds.

FIG. 5A is a histogram showing the level of DPP-8 activity in vitro following exposure to Val-boroPro and Glu-boroPro.

FIG. 5B is a graph showing the level of DPP-8 activity in vitro as a function of time after exposure to the indicated amino acid boroPro compounds.

FIG. 6A is a graph showing the level of blood glucose in vivo as a function of time following administration of Glu-boroPro and an oral glucose challenge.

FIG. 6B is a histogram showing the level of area under the curve (AUC) following in vivo exposure to Glu-boroPro.

FIG. 7A is a graph showing the level of DPP-IV activity in vivo as a function of time immediately following administration of Glu-boroPro and an oral glucose challenge.

FIG. 7B is a graph showing the level of DPP-IV activity in vivo as a function of time (longer time interval) following administration of Glu-boroPro and an oral glucose challenge.

FIG. 7C is a graph showing the level of blood glucose in vivo as a function of time following administration of Glu-boroPro and an oral glucose challenge.

FIG. 7D is a graph showing the level of insulin in vivo as a function of time following administration of Glu-boroPro and an oral glucose challenge.

FIG. 7E is a graph showing the level of GLP-1 (1-36) in vivo as a function of time following Glu-boroPro and an oral glucose challenge.

FIG. 8 is a schematic showing the study design of the effect of Glu-boroPro on glycemic control in male Zucker diabetic (ZDF) rats.

FIG. 9A is a graph showing the level of HbA₁C in ZDF rats as a function of time following administration of vehicle (control), NVPLAF237 (an anti-diabetic agent), and Glu-boroPro.

FIG. 9B is a histogram showing the level of HbA₁C in ZDF rats at day 46 following administration of vehicle (control), NVPLAF237 (an anti-diabetic agent), and Glu-boroPro.

FIG. 10 is a graph showing the amount of food intake as a function of time in ZDF rats following administration of vehicle (control), NVPLAF237 (an anti-diabetic agent), and Glu-boroPro.

FIG. 11A is a graph showing the 24-hour glucose profile as a function of time in ZDF rats following administration of vehicle (control), NVP-LAF237 (an anti-diabetic agent), and Glu-boroPro.

FIG. 11B is a histogram showing the AUC (area under the curve) 24-hour glucose profile on day 14-15 in ZDF rats following administration of vehicle (control), NVPLAF237 (an anti-diabetic agent), and Glu-boroPro.

FIG. 12 is a graph showing the OGTT (oral glucose tolerance test) glucose profile as a function of time in ZDF rats at day 45 following administration of vehicle (control), NVPLAF237 (an anti-diabetic agent), and Glu-boroPro.

FIG. 13 is a histogram showing the insulin sensitivity index in ZDF rats following administration of vehicle (control), NVPLAF237 (an anti-diabetic agent), Metformin, and Glu-boropro.

FIG. 14 is a set of histograms showing changes in baseline glucose, free fatty acids (FFA), cholesterol, and triglycerides (TG) in ZDF rats following administration of vehicle (control), NVPLAF237 (an anti-diabetic agent), Metformin, and Glu-boroPro.

FIG. 15 is a graph showing the insulin (A) and the GLP-1 (B) responses as a function of time in ZDF rats following administration of vehicle (control), NVPLAF237 (an anti-diabetic agent), and Glu-boroPro.

FIG. 16A is a graph showing the OGTT (oral glucose tolerance test) plasma insulin profile as a function of time in DIO rats following administration of vehicle (control), NVPLAF237 (an anti-diabetic agent), and Glu-boroPro.

FIG. 16B is a histogram showing the AUC (area under the curve) plasma insulin in DIO rats following administration of vehicle (control), NVPLAF237 (an anti-diabetic agent), and Glu-boroPro.

FIG. 17A is a graph showing the OGTT (oral glucose tolerance test) plasma insulin profile as a function of time in ZDF rats 17 hours following administration of vehicle (control), NVP-LAF237 (an anti-diabetic agent), and Glu-boroPro.

FIG. 17B is a histogram showing the AUC (area under the curve) plasma insulin in ZDF rats 17 hours following administration of vehicle (control), NVPLAF237 (an anti-diabetic agent), and Glu-boroPro.

It is to be understood that the drawings are not required for enablement of the invention.

### DETAILED DESCRIPTION OF THE INVENTION

The invention relates, in part, to the treatment and prevention of conditions that are associated with abnormal glucose metabolism, such as abnormal glucose tolerance, absorption, metabolism, utilization and the like. These conditions are referred to as glucose-associated conditions.

The invention also relates to the treatment and prevention of conditions that are not necessarily associated with abnormal glucose metabolism (i.e., conditions that are not necessarily glucose-associated). A condition that is not necessarily a glucose-associated condition is a condition that may develop in the presence of normal glucose metabolism (or conversely in the absence of abnormal glucose metabolism). Such conditions include, for example, metabolic syndrome, dyslipidemias, cardiovascular disorders, and some forms of obesity. The compounds of the invention are also useful in lowering triglyceride levels, free fatty acids, C- reactive protein (CRP), HbA₁C, total glycosylated hemoglobin (TGHb or HbA,), in increasing insulin sensitivity index, and stimulating insulin release.

Glucose-associated conditions include but are not limited to type 1 diabetes (insulin dependent diabetes mellitus or IDDM), type 2 diabetes (non-insulin dependent diabetes mellitus or NIDDM), gestational diabetes, diabetic complications such as metabolic acidoses (e.g., diabetic ketoacidosis (DKA)), carbohydrate and lipid metabolism abnormalities, glucosuria, micro- and macrovascular disease, polyneuropathy and diabetic retinopathy, diabetic nephropathy (e.g., albuminuria), insulin resistance, impaired glucose tolerance (or glucose intolerance), obesity, hyperglycemia (elevated blood glucose concentration), hyperinsulinemia, hyperlipidemia, hyperlipoproteinemia, atherosclerosis and hypertension (high blood pressure) related thereto, and various metabolic disorders. Metabolic disorders include digestive tract diseases such as ulceric or inflammatory disease; congenital or acquired digestion and absorption disorder including malabsorption syndrome; disease caused by loss of a mucosal barrier function of the gut; and protein-losing gastroenteropathy. Ulceric diseases include gastric ulcer, duodenal ulcer, small intestinal ulcer, colonic ulcer and rectal ulcer. Inflammatory diseases include esophagitis, gastritis, duodenitis, enteritis, colitis, Crohn's disease, proctitis, gastrointestinal Behcet, radiation enteritis, radiation colitis, radiation proctitis, enteritis and medicamentosa. Malabsorption syndrome includes essential malabsorption syndromes such as disaccharide-decomposing enzyme deficiency, glucose-galactose malabsorption, fructose malabsorption; secondary malabsorption syndrome, short gut syndrome, cul-de-sac syndrome; and indigestible malabsorption syndromes such as syndromes associated with resection of the stomach, e.g., dumping syndrome. Other conditions associated with above-normal blood glucose concentration either in an acute or chronic form are also embraced by the invention. The invention also intends to embrace treatment of conditions which would benefit from beta cell preservation, reduced glucagon levels or increased insulin availability.

Diabetes is generally a disease in which the body is not able to produce or does not adequately utilize insulin. Insulin is a hormone that facilitates entry of sugars, starches, and the like into cells, thereby allowing their conversion into useable energy for the body. In diabetes, therefore, there is a buildup of glucose in the blood due to the inefficient or nonexistent cellular uptake of sugar, starches and the like. Type 2 diabetes is also characterized by progressive beta-cell failure. Type 2 diabetes is also referred to as adult onset diabetes or non-insulin-dependent diabetes (NIDDM).

It was found according to the invention that Glu-boroPro exhibited a combination of potency and duration of DPP-IV inhibition that was significantly better than that of other known amino boronic dipeptides. This difference in activity between the amino boronic dipeptides tested was surprising because the compounds are structurally similar and behave relatively equivalently in other assays (e.g., DPP-IV inhibition). The significance of Glu-boroPro in the treatment of type 2 diabetes and other glucose-associated conditions was indicated in rodent models in which the compound was shown to control blood glucose levels and stimulate insulin and GLP-1 [7-36] levels following oral glucose challenge. These assays provide surrogate readouts that parallel anti-diabetic activity in vivo. Glu-boroPro also demonstrated suitable pharmacological properties and specificity of action, making it even more appropriate for in vivo use in the management of glucose-associated conditions such as type 2 diabetes.

The potential of Glu-boroPro to treat conditions that are not necessarily glucose-associated conditions was indicated in in vivo rodent experiments in which the compound was shown to lower triglycerides, free fatty acids, and HbA₁C, and total glycosylated hemoglobin (TGHb), to improve 24-hour glucose profiles, to increase insulin sensitivity index, to stimulate insulin release, and to reduce food intake.

Although not intending to be bound by any particular mechanism or theory, DPP-IV is presumed to be the target of Glu-boroPro containing compounds. DPP-IV is responsible for the rapid N-terminal degradation of GIP and GLP-1 (t_{1/2}˜1 min) in vivo (4). DPP-IV is therefore a molecular target for compounds designed to amplify the biological activity of GLP-1 and GIP (4). Because resistance to the activity of GIP appears to develop in conditions such as glucose-associated conditions (e.g., type 2 diabetes), it is currently thought that inhibition of DPP-IV will mainly impact the activity of GLP-1. Because GLP-1 is an incretin that stimulates insulin production by pancreatic β-cells in response to the oral intake of glucose (7), DPP-IV plays a physiological role in the regulation of blood glucose levels. This has been validated by the absence of N-terminal degradation of GLP-1 and enhanced insulin secretion in response to oral glucose challenge in DPP-IV-null mice generated by homologous recombination (8). GLP-1 also inhibits glucagon synthesis and gastric emptying, promotes the growth of pancreatic islets and β-cells, and may have an anorexic effect by acting on the hypothalamus. DPP-IV inhibitors may amplify these other biological activities of GLP-1. As a result, the invention embraces methods for inducing weight loss or preventing weight gain, particularly in obese subjects regardless of whether such subjects are diabetic or not.

The invention provides compositions and methods of use of Glu-boroPro containing compounds. Glu-boroPro containing compounds, as used herein, include Glu-boroPro or compounds that are converted (via enzymatic, chemical, metabolic, or any other means, in vivo or ex vivo) to Glu-boroPro. Glu-boroPro containing compounds include prodrugs which, as described further below, are compounds that when acted upon in vivo release Glu-boroPro.

Glu-boroPro is a compound that contains a glutamic acid residue bound via a carboxy (C) terminal bond to a pyrrolidine which in turn is bound to a boronic acid or a boronic ester. For the sake of convenience and brevity, various aspects and embodiments of the invention refer to Glu-boroPro but it is to be understood that other Glu-boroPro containing compounds (e.g., prodrugs) are also embraced by the invention and can be equivalently used in the aspects and embodiments described.

Glu-boroPro has a structure as follows: 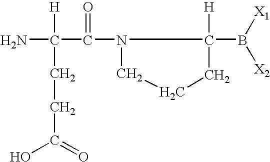
wherein each X₁ and X₂ is, independently, a hydroxyl group or a group capable of being hydrolyzed to a hydroxyl group in aqueous solution at physiological pH. The bond between the carbon in the pyrrolidine ring and the boron can be in an S-configuration, but it is preferably in the R-configuration. The peptide bond between the glutamic acid residue and the pyrrolidine can be in the R-configuration, but in some embodiments it is preferably in the S-configuration. In some embodiments X₁ and X₂ are hydroxyl groups.

Accordingly, the compound can have the following structure showing an S-R configuration (i.e., the glutamic acid residue to pyrrolidine bond is in the S-configuration and the carbon to boron bond is in the R-configuration):

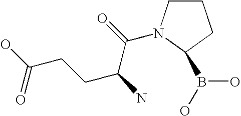

Glu-boroPro therefore includes L-Glu-R-boroPro, D-Glu-R-boroPro, L-Glu-S-boroPro and D-Glu-S-boroPro.

Glu-boroPro can also be provided in cyclic form, which is then converted into a linear form upon in vivo administration, particularly once exposed to an acidic environment such as the stomach. Cyclic amino boronic acids are described in greater detail in U.S. Pat. No. 6,355,614 B1, issued Mar. 12, 2002, the entire contents of which are incorporated by reference herein. The linear and cyclic forms of Glu-boroPro are provided in solution or dry form. Linear and cyclic forms of Glu-boroPro may be in equilibrium. A cyclic Glu-boroPro can have the following structure:

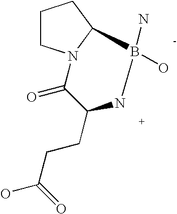

Another class of Glu-boroPro containing compounds comprises Glu-boroPro bound to additional amino (N) terminal naturally or non-naturally occurring amino acid residues, peptides or peptidomimetics. A general formula for this class of compounds is 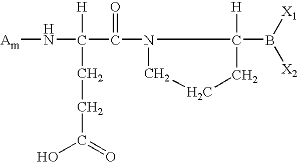
wherein A is any naturally or non-naturally occurring amino acid, peptide or peptidomimetic bonded in either an S- or an R-configuration, m is an integer from 0-100 or more, such that when m is greater than one and A is an amino acid residue, each A in Aₘ may be a different amino acid residue from every other A in Aₘ; wherein when A is a peptide or a peptidomimetic m=1; and each X₁ and X₂ is, independently, a hydroxyl group or a group capable of being hydrolyzed to a hydroxyl group in aqueous solution at physiological pH. The C bonded to B can be an S-configuration but preferably it is an R-configuration. In some important embodiments the peptide bonds between amino acids are in the S-configuration. If such peptide bonds include serine or cysteine, then such bond may be in the R-configuration. In some embodiments X₁ and X₂ are hydroxyl groups.

Depending on the embodiment, m is an integer from 0-30, or an integer from 0-20, or an integer from 0-10. In some important embodiments m is 1, 2, 3, 4, 5, 6, 7, 8, 9, 10, or it is a multiple of two (e.g., 2, 4, 6, 8, 10, etc.), such as a repeating dipeptide having a proline residue at the C terminal (e.g., A-Pro). In some preferred embodiments the general formula for such compounds is 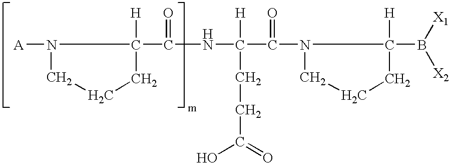
wherein A is any naturally or non-naturally occurring amino acid in an S- or an R-configuration; m is an integer from 0-100 or more, provided that A in each repeating bracketed unit can be a different amino acid residue; and each X₁ and X₂ is, independently, a hydroxyl group or a group capable of being hydrolyzed to a hydroxyl group in aqueous solution at physiological pH. In some embodiments the glutamic acid chiral center is in the S-configuration. Glu-boroPro can also be attached to 3, 5, 6, 7, 9, 12 etc. amino acid residues.

Glu-boroPro containing compounds (including Glu-boroPro) in some instances may be substantially optically pure. That is, at least 90%, 92%, 94%, 95%, 96%, 97%, 98% or 99% of the carbon atoms bearing boron are of the R-configuration in some embodiments.

A synthesis scheme for making the enantiomers of the invention is as follows:

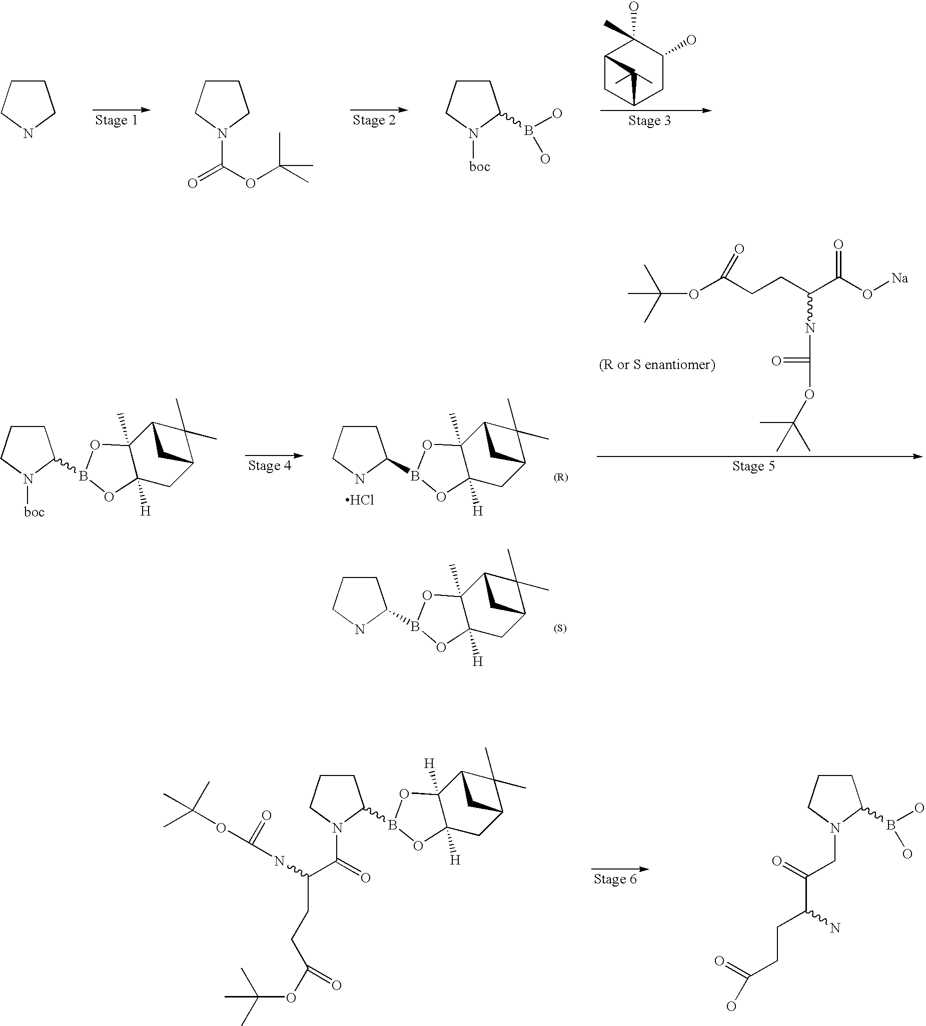

Further methods for synthesizing of these agents are disclosed in Coutts et al. J. Med. Chem., 1996, 39:2087-2094 and in published PCT application W093/10127, published May 27, 1993 and in published PCT application WO 93/08259. As will be understood to those of ordinary skill in the art, the compounds of the invention can be synthesized using D- and preferably L- isomers of glutamic acid and proline.

Glu-boroPro containing compounds also embrace prodrugs of Glu-boroPro. A prodrug of Glu-boroPro as used herein is a compound that is metabolized in vivo to Glu-boroPro or disintegrates (e.g., upon contact with stomach acid) to form Glu-boroPro. Some prodrugs are converted into Glu-boroPro via hydrolysis or oxidation in vivo. These include alcohol precursors of Glu-boroPro that are oxidized in vivo (e.g., in the liver) and that have the following structures: 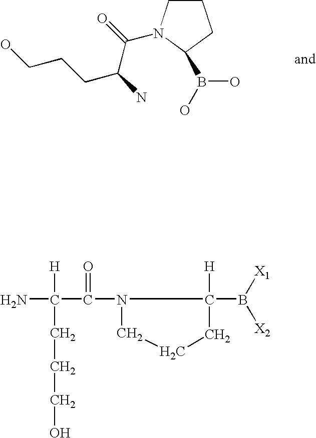
and a boroxine derivative of Glu-boroPro having the following structure: where 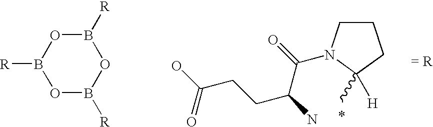
as well as esters of Glu-boroPro and related compounds. Prodrugs of Glu-boroPro also include cyclized versions of the molecule, as discussed above.

Another category of prodrugs includes compounds that are converted to Glu-boroPro by enzymes such as post-prolyl cleaving enzymes (e.g., DPP-IV). However, the invention is not so limited and other prodrugs are also contemplated including those converted to Glu-boroPro by non-post-prolyl cleaving enzymes. Examples of suitable prodrug moieties are disclosed in U.S. Pat. Nos. 5,462,928 issued Oct. 31, 1995; and 6,100,234 issued Aug. 8, 2000; and published PCT applications WO 91/16339 published Oct. 31, 1991; WO 93/08259 published Apr. 29, 1993; and WO 03/092605, published Nov. 13, 2003, among others.

The length of such prodrug compounds may be 4, 6, 8, 10, 12, 14, 16, 18, 20, 30, 50, 100 or more residues in length (whereby the length includes the glutamic acid and proline residues). Multiples of 3 are also contemplated.

The amino acid residues may be amino acid in nature (including naturally and non-naturally occurring amino acids). Examples of naturally occurring amino acids are glycine (Gly), and the L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartic acid (Asp), glutamic acid (Glu), asparagine (Asn), glutamine (Gln) and proline (Pro). Non-naturally occurring amino acids include the D-forms of Ala, Val, Leu, Ile, Phe, Tyr, Trp, Cys, Met, Ser, Thr, Lys, Arg, His, Asp, Glu, Asn, Gln, and Pro.

Examples of non-naturally occurring amino acids include but are not limited to 4-hydroxy-proline (Hyp), 5-hydroxy-lysine, norleucine (Nle), 5-hydroxynorleucine (Hyn), 6-hydroxynorleucine, ornithine, cyclohexylglycine (Chg), N-Methylglycine (N-MeGly), N-Methylalanine (N-MeAla), N-Methylvaline (N-MeVal), N-Methylleucine (N-MeLeu), N-Methylisoleucine (N-MeIle), N-Methylnorleucine (N-MeNle), N-Methyl-2-aminobutyric acid (N-MeAbu) and N-Methyl-2-aminopentanoic acid (N-MeNva), methylthreonine, nitroglutamine, norleucine (Nle), norvaline, ornithine, phosphoserine, pipecolic acid, sarcosine, taurine, tert-leucine, thiazolidine carboxylic acid, thyroxine, trans-4-hydroxyproline, and trans-3-methylproline.

Non-naturally occurring amino acids also include, for example, beta-amino acids. Non-naturally occurring amino acids also include alpha-amino acids wherein the side chains are replaced with synthetic derivatives. Representative side chains of naturally occurring and non-naturally occurring a-amino acids are shown below in Table 1.

TABLE 1

CH₃—
HO—CH₂—
C₆H₅—CH₂—

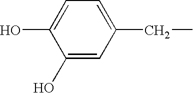

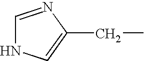

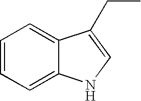

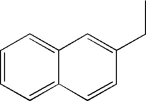

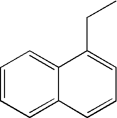

HS—CH₂—
HO₂C—CH(NH₂)—CH₂—S—S—CH₂—
CH₃—CH₂—
CH₃—S—CH₂—CH₂—
CH₃—CH₂—S—CH₂—CH₂—
HO—CH₂—CH₂—

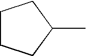

CH₃—CH(OH)—
HO₂C—CH₂—NHC(═O)—CH₂—
HO₂C—CH₂—CH₂—
NH₂C(═O)—CH₂—CH₂—
(CH₃)₂—CH—
(CH₃)₂—CH—CH₂—
CH₃—CH₂—CH₂—
H₂N—CH₂—CH₂—CH₂—
H₂N—C(═NH)—NH—CH₂—CH₂—CH₂—
H₂N—C(═O)—NH—CH₂—CH₂—CH₂—
CH₃—CH₂—CH(CH₃)—
CH₃—CH₂—CH₂—CH₂—
H₂N—CH₂—CH₂—CH₂—CH₂—

Both D, L, and racemic configurations of hydrophobic amino acids can be employed. Suitable hydrophobic amino acids can also include amino acid analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C I-C8 hydrocarbons which are completely saturated, which contain one or two heteroatoms such as nitrogen, oxygen or sulfur and/or which contain one or more units of desaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F) —O (aliphatic, substituted aliphatic, benzyl, substituted benzyl, aryl or substituted aryl group), —CN, —NO₂, —COOH, —NH₂, —NH (aliphatic group, substituted aliphatic, benzyl, substituted benzyl, aryl or substituted aryl group), —N (aliphatic group, substituted aliphatic, benzyl, substituted benzyl, aryl or substituted aryl group)₂, —COO (aliphatic group, substituted aliphatic, benzyl, substituted benzyl, aryl or substituted aryl group), —CONH₂, —CONH (aliphatic, substituted aliphatic grou p, benzyl, substituted benzyl, aryl or substituted aryl group)), —SH, —S (aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lypophilicity or hydrophobicity of natural amino acids which are hydrophilic.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

As mentioned above, the specification focuses on Glu-boroPro containing compounds as exemplary agents to be used in the invention. It is to be understood however that other reactive moieties can be used in place of the boronic acid reactive group. These include but are not limited to phosphonates such as organo phosphonates and peptidyl (alpha-arninoalkyl) phosphonate esters, fluoroalkylketones, alphaketo amides, alphaketo esters, alphaketo acids, N-peptiolyl-O-acylhydroxylamines, azapeptides, azetidines, fluoroolefins dipeptide isoesters, cyanopyrrolidines, aminoacyl pyrrolidine-2-nitriles and thiazolides such as 4-cyanothiazolidides.

In addition or in place of amino acids, the agents of the invention may also be comprised of saccharides, fatty acids, sterols, isoprenoids, purines, pyrimidines, derivatives or structural analogs of the above, peptoids, random bio-oligomers (U.S. Pat. No. 5,650,489), benzodiazepines, diversomeres such as dydantoins, nonpeptidyl peptidomimetics with a beta-D-glucose scaffolding, oligocarbamates, or combinations thereof and the like. Many, if not all, of these compounds can be synthesized using recombinant or chemical library approaches. A vast array of compounds can be generated from libraries of synthetic or natural compounds.

The methods provided herein embrace treatment methods. As used herein, the term “treatment” refers to the administration of one or more therapeutic agent to a subject for the purpose of achieving a medically desirable benefit. Accordingly, “treatment” intends to embrace both “prophylactic” and “therapeutic” treatment methods. Prophylactic treatment methods refer to treatment administered to a subject at risk of developing a condition. For example, a prophylactic treatment for a glucose-associated condition such as type 2 diabetes refers to treatment of a subject at risk of developing type 2 diabetes (e.g., a prediabetic subject). Therapeutic treatment refer to treatment of a subject after the diagnosis of such a condition.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops), provided that it would benefit from the methods provided herein. Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. In all embodiments human subjects are preferred. Human subjects can be subjects at any age, including adults, juveniles, infants and fetuses in utero. Pregnant subjects such as pregnant human subjects are also contemplated.

One category of subjects to be treated according to the invention are those with impaired glucose tolerance (or glucose intolerance), such as but not limited to subjects having or at risk of developing type 2 diabetes. These subjects generally demonstrate an inability to control glucose levels upon eating, as would a non-diabetic or non-prediabetic “normal” subject. Subjects at risk of developing type 2 diabetes who demonstrate impaired glucose tolerance are considered to be in a prediabetic state. Glucose tolerance can be measured using glucose challenge tests. There are at least two such tests currently available: the Fasting Plasma Glucose Test (FPG) and the Oral Glucose Tolerance Test (OGTT). In human subjects, a FPG blood glucose level between 100-126 mg/dl of blood is indicative of a prediabetic state and an FPG blood glucose level equal to or greater than 126 mg/dl of blood is indicative of diabetes. OGTT measures blood glucose level two hours after ingestion of a glucose-rich drink (which itself occurs after a fasting period). An OGTT blood glucose level between 140-199 mg/dl is indicative of prediabetes, and a level equal to or greater than 200 mg/dl is indicative of diabetes. The presence of glycosylated hemoglobin at levels equal to or greater than 7.0% is also considered an early indicator of the onset of diabetes.

Risk factors for type 2 diabetes include obesity, family history of diabetes, prior history of gestational diabetes, impaired glucose tolerance (as discussed above), physical inactivity, and race/ethnicity.

Subjects at risk of developing diabetes also may be overweight to the point of being obese. The state of being overweight or obese is defined in terms of the medically recognized body mass index (BMI). BMI equal to a person's body weight (kg) divided by the square of his or her height in meters (i.e., wt/(ht)²). A subject having a BMI of 25 to 29.9 is considered overweight. A subject having a BMI of 30 or more is considered obese.

Symptoms associated with diabetes include but are not limited to frequent urination, excessive thirst, extreme hunger, unusual weight loss, increased fatigue, irritability and blurred vision.

Diabetes is associated with other conditions, many of which result from a diabetic state. These include acute metabolic complications such as diabetic ketoacidosis and hyperosmolar coma, and late complications such as circulatory abnormalities, retinopathy, nephropathy, neuropathy and foot ulcers. A more detailed description of the foregoing terms can be obtained from a number of sources known in the art (see, e.g., Harrison's Principles of Internal Medicine, 15^{th} Edition, McGraw-Hill, Inc., N.Y.). Thus, the methods of the invention also embrace ameliorating or resolving diabetes-associated conditions such as but not limited to those recited above.

Another category of subjects to be treated according to the invention are subjects with metabolic syndrome. Metabolic syndrome (also referred to as syndrome X) is a cluster of risk factors that is responsible for increased cardiovascular morbidity and mortality. The National Cholesterol Education Program—Adult Treatment panel (NECP-ATP III) identified metabolic syndrome as an independent risk factor for cardiovascular disease. (National Institutes of Health: Third Report of the National Cholesterol Education Program Expert Panel on Detection, Evaluation, and Treatment of High Blood Cholesterol in Adults (Adult Treatment Panel III). Executive publication no. 01-3670). As used herein, metabolic syndrome is defined as the co-occurrence of any three of the abnormalities in Table 2.

TABLE 2
Diagnostic Criteria for Metabolic Syndrome

| | Diagnostic criteria (three of the |
|---|---|
| Component | following) |
| Abdominal/central obesity | Waist circumference: >102 cm (40 in.) |
| | in men, >88 cm (35 in.) in |
| | women |
| Elevated triglycerides | > or =150 mg per dL |
| Low HDL cholesterol | <40 mg per dL (<1.036 mmol per L) |
| | for men, <50 mg per dL (<1.295 mmol |
| | per L) for women |
| High blood pressure | > or =130/85 mm Hg or documented |
| | use of antihypertensive therapy |
| High fasting glucose | > or =110 mg per dL (> or =6.1 mmol |
| | per L) |

Thus, a subject may have metabolic syndrome without diabetes or an abnormal glucose metabolism.

Another category of subjects to be treated according to the invention are subjects with cardiovascular disorders. “Cardiovascular disorder”, as used herein, includes elevated blood pressure, atherosclerosis, heart failure or a cardiovascular event such as acute coronary syndrome, myocardial infarction, myocardial ischemia, chronic stable angina pectoris, unstable angina pectoris, angioplasty, stroke, transient ischemic attack, claudication(s), or vascular occlusion(s).

Risk factors for a cardiovascular disorder include dyslipidemia, obesity, diabetes mellitus, pre-hypertension, elevated level(s) of a marker of systemic inflammation, age, a family history of cardiovascular disorders, and cigarette smoking. The degree of risk of a cardiovascular disorder or a cardiovascular event depends on the multitude and the severity or the magnitude of the risk factors demonstrated by the subject. Risk charts and prediction algorithms are available for assessing the risk of cardiovascular disorders and cardiovascular events in a human subject based on the presence and severity of risk factors. One such example is the Framingham Heart Study risk prediction score. For example, the human subject is at an elevated risk of having a cardiovascular event if the subject's 10-year calculated Framingham Heart Study risk score is greater than 5%, 6%, 7%, 8%, 9%, 10%, 11%, 12%, 13%, 14%, 15%, 16%, 17%, 18%, 19%, or 20%.

Another method for assessing the risk of a cardiovascular event in a human subject is a global risk score that incorporates a measurement of a level of another marker of systemic inflammation (e.g. CRP) or a risk factor into the Framingham Heart Study risk prediction score. Other methods of assessing the risk of a cardiovascular disorder or cardiovascular event in a human subject include coronary calcium scanning, cardiac magnetic resonance imaging, and magnetic resonance angiography.

Another category of subjects to be treated according to the invention are subjects with dyslipidemias. As used herein, dyslipidemia is an abnormal serum, plasma, or blood lipid profile in a subject. An abnormal lipid profile may be characterized by total cholesterol, low density lipoprotein (LDL)-cholesterol, triglyceride, apolipoprotein (apo)-B or Lp(a) levels above the 90^{th} percentile for the general population or high density lipoprotein (HDL)-cholesterol or apo A-1 levels below the 10^{th} percentile for the general population. Dyslipidemia can include hypercholesterolemia and/or hypertriglyceridemia. Hypercholesterolemic human subjects and hypertriglyceridemic human subjects are associated with increased incidence of cardiovascular disorders. A hypercholesterolemic human subject is one who fits the current criteria established for a hypercholesterolemic human subject. A hypercholesterolemic subjwect has an LDL cholesterol level of >160 mg/dL, or >130 mg/dL and at least two risk factors selected from the group consisting of male gender, family history of premature coronary heart disease, cigarette smoking, hypertension, low HDL (<35 mg/dL), diabetes mellitus, hyperinsulinemia, abdominal obesity, high lipoprotein, and personal history of a cardiovascular event. A hypertriglyceridemic human subject is one who fits the current criteria established for a hypertriglyceridemic subject. A hypertriglyceridemic human subject has a triglyceride (TG) level of >200 mg/dL.

Dyslipidemias encompassed by this invention include dyslipidemias caused by single gene defects, dyslipidemias that are multifactorial or polygenic in origin, as well as dyslipidemias that are secondary to other disease states or secondary to pharmacological agents. Examples of genetic dyslipidemias include Familial Hypercholesterolemia, Familial Defective Apo B 100, Familial Hypertriglyceridemia, Familial Apoprotein CII deficiency, Hepatic Lipase Deficiency, Familial Combined Hyperlipidemia, Dysbetalipoproteinemia, and Familial Lipoprotein Lipase Deficiency.

One example of multifactorial or polygenic dyslipidemia is Polygenic hypercholesterolemia.

Other examples of dyslipidemias include Hyperlipidemia Type I, II, III, IV, and V (also called Hyperlipoproteinemia Type I, II, III, IV, and V respectively).

Dyslipidemias that are secondary to other disease states include dyslipidemias secondary to diabetes mellitus, hypothyroidism, renal disease (such as for example, nephrotic syndrome and renal failure), liver disease (such as, for example, primary biliary cirrohsis, and extrahepatic obstruction) and Acquired Immunodeficiency Syndrome (AIDS).

Also encompassed by the invention are subjects with Hyperlipidemia Type I, II, III, IV and/or V (preferrably adults with Hyperlipidemia Type IV and/or V) who are at risk for pancreatitis.

A subject at risk for pancreatitis is a subject who has risk factors for pancreatitis. Examples of risk factors for pancreatitis include hypertriglyceridemia, hypercalcemia, gallstones, alcohol use, alcoholism, viral infection(s) (for example, mumps, hepatitis, and Epstein-Barr virus), bacterial infection(s), mycoplasma infections, campylobacter infections, structural abnormalities of the pancreas or the common bile duct, abdominal trauma, surgery (e.g. abdominal surgery, pancreatic cancer, penetrating peptic ulcer, renal failure, inherited diseases (for example, cystic fibrosis), genetic abnormalities, vascular diseases such as vasculitis, and intake of certain drugs (such as, for example, corticosteroids, non-steroidal anti-inflammatory disorders, thiazides, furosemide, azathioprine, angiotensin-converting enzyme inhibitors, estrogens, pentamidine, valproic acid, and antibiotics (such as, for example, tetracyclines and sulfonamides)). Risk factors for pancreatitis are known to those of ordinary skill in the art and are described in medical textbooks such as Harrison's Principles of Internal Medicine, 15^{th} Edition, McGraw-Hill, Inc., N.Y.

The subject with dyslipidemia may or may not respond to diet. As used herein, a response to diet is a response to a change or modification in eating habit(s) or behavior(s) without the use of pharmacological agent(s). A response to diet in a subject is a decrease in the level of total cholesterol, low density lipoprotein (LDL)-cholesterol, triglyceride, apolipoprotein (apo)-B and/or Lp(a) by at least 5%, 10%, 15%, 20%, 25%, 30%, 35%, 40%, 45%, 50%, 55%, 60%, 65%, 70%, 75%, or more from the level before the start of the diet. In the alternative, a response to diet is an increase in the level of high density lipoprotein (HDL)-cholesterol and/or apo A-1 levels by at least 5%, 10%, 15%, 20%, 25%, 30%, 35%, 40%, 45%, 50%, 55%, 60%, 65%, 70%, 75%, or more, from the level before the start of the diet. Also, in the alternative, a response to diet is a decrease in the triglyceride level in the blood to at least 250 mg/dl, 245 mg/dl, 240 mg/dl, 235 mg/dl, 230 mg/dl, 225 mg/dl, 220 mg/dl, 215 mg/dl, 210 mg/dl, 205 mg/dl, 200 mg/dl, 195 mg/dl, 190 mg/dl, 185 mg/dl, 180 mg/dl, 175 mg/dl, 170 mg/dl, 165 mg/dl, 160 mg/dl, 155 mg/dl, 150 mg/dl, 145 mg/dl, 140 mg/dl, or less.

In some embodiements, the dyslipidemia is caused by a pharmacological agent(s). Examples of pharmacological agents that cause dyslipidemias include, but are not limited to, ethanol, progestogens, estrogens, isotretinoin, glucocorticoids, bile acid-bonding resins, thiazides, protease inhibitors cyclosporine, thiazides, beta-blockers, and anabolic steroids.

Subjects at risk of developing a dyslipidemia are also encompassed by this invention. Such subjects include subjects with Familial Hypertriglyceridemia, Familial Apoprotein CII deficiency, Hepatic Lipase Deficiency, Familial Combined Hyperlipidemia, Dysbetalipoproteinemia, and Familial Lipoprotein Lipase Deficiency. Subjects having or at risk of developing a dyslipidemia also include subjects who suffer from alcohol abuse or dependence, pancreatitis, glucose-6-phosphatase deficiency, hepatitis, Systemic Lupus Erythematosus (SLE), monoclonal gammopathies (such as, for example, multiple myeloma and lymphomas), and Acquired Immunodeficiency Syndrome (AIDS).

Subjects with elevated blood pressure (hypertension) as well as subjects with pre-hypertension are also encompassed by the invention. Elevated blood pressure or hypertension is defined as a systolic blood pressure >120 mm Hg, or a diastolic pressure >80 mm Hg or an elevation of both (i.e., systolic blood pressure >120 mm Hg and a diastolic pressure >80 mm Hg). Pre-hypertension is defined as systolic blood pressure between 115 and 120 mm Hg, and/or a diastolic pressure between 75 and 80 mm Hg.

Subjects with elevated levels of C-reactive protein (CRP) may be treated according to this invention. Elevated levels of CRP, a marker of systemic inflammation, have been described among human subjects with acute ischemia or myocardial infarction, and predict episodes of recurrent ischemic cardiovascular events. Elevated levels of CRP have also been associated with risk of myocardial infarction among human subjects. Elevated levels of CRP were determined to be predictive of future cardiovascular events in otherwise healthy human subjects. The predictive capacity of CRP was also determined to be independent of the predictive capacity of lipids such as cholesterol. Recent studies suggest that lowering CRP to a target level leads to a lowering of the risk of future cardiovascular events. A desired target level for CRP to lower the risk of a future cardiovascular event(s) in a human subject is <1 mg/L. A CRP level of >1 mg/L is considered a risk factor for a future cardiovascular event(s).

Another category of subjects to be treated according to the invention are subjects with inflammation and inflammatory disorders. Inflammation and inflammatory disorders are characterized by activation of the immune system in a tissue or an organ to abnormal levels that may lead to abnormal function and/or disease in the tissue or organ. Several markers of inflammation are available to measure activation of the immune system. Examples of markers of inflammation include CRP, soluble intercellular adhesion molecule (sICAM-1), ICAM 3, BL-CAM, LFA-2, VCAM-1, NCAM, PECAM, fibrinogen, serum amyloid A (SAA), lipoprotein associated phospholipase A2 (LpP1A2), sCD40 ligand (sCD40L), myeloperoxidase, Interleukin-6 (IL-6), and Interleukin-8 (IL-8).

Subjects with low levels of adiponectin may be treated according to this invention. Adiponectin is a protein hormone produced and secreted by adipocytes (fat cells). Adiponectin influences the body's response to insulin and regulates the metabolism of lipids and glucose. Adiponectin also has anti-inflammatory effects on the cells lining the walls of blood vessels. High blood levels of adiponectin are associated with a reduced risk of heart attack. A desired target plasma level for adiponectin in a human subject is >5 μg/mL. A more desired target plasma level for adiponectin in a human subject is >10 μg/mL.

Presently there are commercial sources which produce reagents for assays for adiponectin. These include, but are not limited to, Phoenix Pharmaceuticals (Belmont, Calif.) Panomic (Redwood, Calif.), and Linco Research (St. Lous, Mo.).

The compounds of the invention are administered in effective amounts. An effective amount is a dosage of the therapeutic agent sufficient to provide a medically desirable result. The effective amount may vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. The dosage may be adjusted by the individual physician in the event of any complication.

An effective amount typically will vary from about 0.001 μg/kg to about 1000 μg/kg, from about 0.01 μg/kg to about 750 μg/kg, from about 0.1 mg/kg to about 500 μg/kg, from about 1.0 μg/kg to about 250 μg/kg, from about 10.0 μg/kg to about 150 μg/kg in one or more dose administrations daily, for one or several days (depending of course of the mode of administration and the factors discussed above). Other suitable dose ranges include 1 μg to 10000 μg per day, 100 μg to 10000 μg per day, 500 μg to 10000 μg per day, and 500 μg to 1000 μg per day. In some particular embodiments the amount is less than 10,000 μtg per day with a range of 750 μg to 9000 μg per day. In one embodiment the effective amount is an amount that does not stimulate cytokine or chemokine induction by the active agent. Although not intending to be bound by any particular theory, the dose of Glu-boroPro containing compound required to stimulate cytokine or chemokine induction may be on the order of 100-fold more than the dose required for treatment according to the methods of the present invention.

As described in greater detail in the Examples, administration of Glu-boroPro leads to, inter alia, inhibition of DPP-IV and to changes in glucose excursion following food intake. The amount of Glu-boroPro containing compound required for treatment according to the invention therefore can also be described in terms of the amount of DPP-IV inhibition. For example, the amount of Glu-boroPro containing compound required to treat glucose-associated conditions such as diabetes may also be the amount that inhibits at least and preferably more than 40%, 50%, 60%, 70%, 80% or 90% of serum DPP-IV, as measured by standard DPP-IV activity assays. The amount of Glu-boroPro containing compound required to treat glucose-associated conditions such as diabetes may also be the amount that reduces a glucose excursion “area under the curve” by about 40-50% relative to a control or untreated subject profile. The “area under the curve” measurement is demonstrated in the Examples and Figures and is a composite measure of the peak and breadth of the glucose profile in a subject, for example, after food intake. Administration of a Glu-boroPro containing compound (e.g., Glu-boroPro) can effect a reduction in the glucose peak and/or in the length of time necessary to recover to a normal level of glucose, for example, after food intake.

A subject having dyslipidemia and treated according to the invention may experience a decrease in the level of total cholesterol, low density lipoprotein (LDL)-cholesterol, triglyceride, apolipoprotein (apo)-B and/or Lp(a) by at least 5%, 10%, 15%, 20%, 25%, 30%, 35%, 40%, 45%, 50%, 55%, 60%, 65%, 70%, 75%, or more of the pre-treatment level. In the alternative, a subject having dyslipidemia and treated according to the invention may experience an increase in the level of high density lipoprotein (HDL)-cholesterol and/or apo A-1 levels by at least 5%, 10%, 15%, 20%, 25%, 30%, 35%, 40%, 45%, 50%, 55%, 60%, 65%, 70%, 75%, or more of the pre-treatment level.

A subject having dyslipidemia and treated according to the invention may experience a change in his/her lipid profile to the “normal ranges” corresponding to 90% of the general population, although treatment is not so limited. Normal ranges of lipid profiles are described in medical textbooks and are known to those of ordinary skill in the art.

A subject having metabolic syndrome and treated according to the invention may, in some embodiments experience a decrease in the levels of the abnormalities of metabolic syndrome from the pre-treatment levels of the abnormalities. In some embodiments the subject may experience a correction of one or more of the abnormalities of metabolic syndrome. In other embodiments the subject may experience a correction of two or more of the abnormalities of metabolic syndrome. In still other embodiments the subject may experience a correction of 3, 4 or all of the abnormalities of metabolic syndrome. Correction of an abnormality associated with metabolic syndrome is achieved when the target of a treatment is reached. Targets of treatment of individual abnormalities of metabolic syndrome are summarized in Table 3.

TABLE 3

| Targeted area | Target |
|---|---|
| Waist circumference | <102 cm (40 in) in men, |
| | <88 cm (35 in) in women |
| Triglycerides | <150 mg per dL |
| HDL cholesterol | >40 mg per dL (<1.036 mmol per L) |
| | for men, |
| | >50 mg per dL (<1.295 mmol per L) |
| | for women |
| Blood pressure | <130/85 mm Hg |
| Fasting glucose | <110 mg per dL (>=6.1 mmol per L) |

In some embodiments the correction of the abnormalities of metabolic syndrome may not involve an improvement or a correction of a glucose abnormality.

The amount of Glu-boroPro containing compound required to treat a cardiovascular disorder in a subject is the amount that will reduce the risk of a future adverse cardiovascular event. Glu-boroPro containing compounds of the invention may be used to prevent cardiovascular disorders or an adverse cardiovascular event (i.e., they can be used prophylactically in human subjects at risk). Thus, an effective amount is that amount which can lower the risk of, slow, or perhaps prevent the development of a cardiovascular disorder or cardiovascular event. It will be recognized that when the Glu-boroPro containing compound is used in acute circumstances, it is used to prevent a medically undesirable result(s) that flow from such adverse events. For example, in the case of myocardial infarction (MI), the Glu-boroPro containing compound may be used to limit injury to the heart as a result of the MI. Targets for treatment for various cardiovascular disorders are described in medical textbooks such as Harrison's Principles of Internal Medicine (15^{th} Edition, McGraw-Hill, Inc., N.Y.)

The amount of Glu-boroPro containing compound required to treat a cardiovascular disorder in a subject is the amount that will reduce the risk of a future adverse cardiovascular event. It should be understood that the Glu-boroPro containing compounds of the invention can be used to prevent cardiovascular disorders (i.e., they can be used prophylactically in human subjects at risk of developing a cardiovascular disorder or an adverse cardiovascular event). Thus, an effective amount is that amount which can lower the risk of, slow or perhaps prevent altogether the development of a cardiovascular disorder or cardiovascular event. It will be recognized that when the Glu-boroPro containing compound is used in acute circumstances, it is used to prevent one or more medically undesirable results that typically flow from such adverse events. For example, in the case of myocardial infarction, the Glu-boroPro containing compound may be used to limit injury to the cardiovascular tissue which develops as a result of the myocardial infarction.

An subject treated according to the invention for weight control or regulation may experience a decrease in his/her weight of at least 5%, 6%, 7%, 8%, 9%, 10 %, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, or more from the pre-treatment weight.

In some embodiments a subject treated according to the invention may experience a decrease in weight of at least 5 lbs, 10 lbs, 15 lbs, 20 lbs, 25 lbs, 30 lbs, 35 lbs, 40 lbs, 45 lbs, 50 lbs, 55 lbs, 60 lbs, 65 lbs, 70 lbs, 75 lbs, 80 lbs, 85 lbs, 90 lbs, 95 lbs, 100 lbs, 105 lbs, 110 lbs, 115 lbs, 120 lbs, 125 lbs, 135 lbs, 135 lbs, 140 lbs, 145 lbs, 150 lbs, or more.

In some embodiments a subject treated according to the invention may experience a decrease in BMI of at least 1, 2, 3, 4, 5, 6, 7, 8, 9, 10, 11, 12, 13, 14, 15, 16, 17, 18, 19, 20, 21, 22, 23, 24, 25 index points, or more.

The subject may also or alternatively experience no weight gain if that is the intended purpose.

The amount of Glu-boroPro containing compound required to increase the insulin sensitivity index in a subject is the amount that will increase insulin sensitivity index in the subject of at least 0.0002, 0.0003, 0.0004, 0.0005, 0.0006, 0.0007, 0.0008, 0.0009, 0.0010, 0.0011, 0.0012, 0.0013, 0.0014, 0.0015, 0.0016, 0.0017, 0.0018, 0.0019, 0.0020, or more index points. The insulin sensitivity index provides a reasonable approximation of whole-body insulin from the OGTT and correlates with the rate of whole-body glucose disposal during the euglycemic insulin clamp. The insulin sensitivity index is: 10,000/square root of [fasting glucose X fasting insulin]×[mean glucose×mean insulin during OGTT]. The insulin sensitivity index is described in more detail by Matsudo and DeFronzo (Diabetes Care. 1999 Sep. 22(9):1462-70).

A subject being treated according to the invention to lower free fatty acid levels may experience a decrease in the level of free fatty acids of at least 5%, 6%, 7%, 8%, 9%, 10%, 11%, 12%, 13%, 14%, 15%, 16%, 17%, 18%, 19%,20%,21%,22%,23%,24%,25%,26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, or more from the pre-treatment level.

A subject being treated according to the invention to lower HbA₁C levels may experience a decrease in the HbA₁C of at least 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, or more from the pre-treatment level of the total HbA₁C. For example, a 1% decrease in the HbA₁C level in a subject with a total HbA₁C level of 7% before the start of treatment would result in a HbA₁C level of 6%.

A subject having or at risk of developing an elevated triglyceride level being treated according to the invention may experience a decrease in the triglyceride level in the blood of the subject of at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, or more from the pre-treatment level.

A subject having or at risk of developing an elevated triglyceride level being treated according to the invention may experience a decrease in the triglyceride level in the blood of the subject of at least 5 mg/dl, 10 mg/dl, 15 mg/dl, 20 mg/dl, 25 mg/dl, 30 mg/dl, 35 mg/dl, 40 mg/dl, 45 mg/dl, 50 mg/dl, 55 mg/dl, 60 mg/dl, 65 mg/dl, 70 mg/dl, 75 mg/dl, 80 mg/dl, 85 mg/dl, 90 mg/dl, 95 mg/dl, 100 mg/dl, 105 mg/dl, 110 mg/dl, 115 mg/dl, 120 mg/dl, 125 mg/dl, 130 mg/dl, 135 mg/dl, 140 mg/dl, 145 mg/dl, 150 mg/dl, or more.

In some embodiments a subject having or at risk of developing an elevated triglyceride level being treated according to the invention may experience a decrease in the triglyceride level in the blood to at least 250 mg/dl, 245 mg/dl, 240 mg/dl, 235 mg/dl, 230 mg/dl, 225 mg/dl, 220 mg/dl, 215 mg/dl, 210 mg/dl, 205 mg/dl, 200 mg/dl, 195 mg/dl, 190 mg/dl, 185 mg/dl, 180 mg/dl, 175 mg/dl, 170 mg/dl, 165 mg/dl, 160 mg/dl, 155 mg/dl, 150 mg/dl, 145 mg/dl, 140 mg/dl, or less.

A subject being treated according to the invention to lower an elevated CRP level may experience a decrease in the CRP level in the blood of the subject of at least 0.2 mg/L, 0.4 mg/L, 0.6 mg/L, 0.8 mg/L, 1.0 mg/L, 1.2 mg/L, 1.4 mg/L,1.6 mg/L, 1.8 mg/L, 2.0 mg/L, 2.2 mg/L, 2.4 mg/L, 2.6 mg/L, 2.8 mg/L, 3.0 mg/L, 3.2 mg/L, 3.4 mg/L, 3.6 mg/L, 3.8 mg/L, 4.0 mg/L, or more.

In some embodiments a subject being treated according to the invention to lower an elevated CRP level to reduce the risk of a future cardiovascular event(s) or to treat inflammation or an inflammatory disorder may experience a decrease in the CRP level in the blood of the subject to at least 3 mg/L, 2.8 mg/L, 2.6 mg/L, 2.4 mg/L, 2.2 mg/L, 2.0 mg/L, 1.8 mg/L, 1.6 mg/L, 1.4 mg/L, 1.2 mg/L, 1.0 mg/L, or less.

In some embodiments a subject being treated according to the invention to lower an elevated CRP level to reduce the risk of a future cardiovascular event(s) or to treat inflammation or an inflammatory disorder may experience a decrease in the CRP level in the blood of the subject of at least 10%, 20%, 30%, 40%, 50%, 60%, 70%, 80%, 90%, 100%, 110%, 120%, 125%, 130%, 135%, 140%, 145%, 150%, 155%, 160%, 170%, 175%, 180%, 185%,190%, 200%, 205%, 210%, 215%, 220%, 225%, 230%, 235%, 240%,245%, 250%, or more from the pre-treatment level.

Presently there are commercial sources which produce reagents for assays for CRP. These include, but are not limited to, Dade-Behring (Deerfield, Illinois), Abbott Pharmaceuticals (Abbott Park, Ill.), CalBiochem (San Diego, Calif.) and Behringwerke (Marburg, Germany).

In some embodiments a subject being treated according to the invention to increase the insulin level in the subject by at least 10%, 20%, 30%, 40%, 50%, 60%, 70%, 80%, 90%, 100%, 110%, 120%, 125%, 130%, 135%, 140%, 145%, 150%, 155%, 160%, 170%, 175%, 180%, 185%, 190%, 200%, 205%, 210%, 215%, 220%, 225%, 230%, 235%, 240%, 245%, 250%, or more from the pre-treatment level.

Unit dosages (i.e., the amount of Glu-boroPro containing compound present in a single dose such as a tablet, pill, capsule and the like) preferably are comparable to the effective amounts shown above. Unit dosages will depend upon how often the agent is administered, whether it is administered together with a second agent, and the route of administration, among other things. As an example, if Glu-boroPro is orally administered to a subject once a day in the absence of a second anti-diabetic agent, then the unit dosage can be approximately 100 μg, approximately 200 μg, approximately 300 μg, approximately 400 μg, approximately 500 μg, approximately 600 μg, approximately 700 μg, approximately 800 μg, approximately 900 μg, or approximately 1000 μg. As used herein, approximately means +/−5%. Alternatively, the unit dosage can be in the range of 100-10000 μg, 500-5000 μg, or 500-1000 μg. In some embodiments the dosage is less than 1000 μg. In other embodiments the unit dosage range is 750-9000 μg. A unit dosage corresponds to the amount of Glu-boroPro containing compound being administered. If Glu-boroPro containing compound is provided as a prodrug, then the amount of total compound administered will be in excess of the unit dosage.

As described in greater detail herein, the invention contemplates administration of Glu-boroPro containing compound(s) and a second agent such as but not limited to an anti-diabetic agent(s). In these aspects and embodiments the dose of the Glu-boroPro containing compound, the second agent, or both the Glu-boroPro containing compound and second agent may be reduced over the dose required when either agent is administered alone. For example, the unit dosage of one or both may be reduced by a factor of 2, 3, 4, 5, 6, 7, 8, 9, 10, 15, 20, 25, 50, 100 or more relative to the unit dosage required when a single agent is administered. The above teaching similarly applies when the second agent is itself a combination of two or more agents (e.g., such as in the case of the anti-diabetic agent Avandamet).

Single or multiple doses of the compounds and/or agents are contemplated. Desired time intervals for delivery of multiple doses can be determined by one of ordinary skill in the art employing no more than routine experimentation. As an example, subjects may be administered two doses daily at approximately 12 hour intervals. Preferably, the compound and/or agent is administered once a day in order to facilitate patient compliance.

The compounds and/or agents may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, once a day, every two days, every three days, every four days, every five days, every six days, every week, every month, or any set number of days or weeks there between. Alternatively, the predetermined routine schedule may involve administration on a daily or twice daily basis for the first week or until a medical benefit is observed, followed by bi-weekly or weekly basis for several months, or longer etc. Any particular combination would be covered by the routine schedule as long as it is determined ahead of time that the appropriate schedule involves administration on a certain day.

Preferably, the compounds and/or agents are designed to be delivered with greatest ease to subjects. This may include for example a once a day oral administration, the timing of which is not dependent upon food intake. Thus, for example, the agent can be taken every morning and/or every evening, regardless of when the subject has eaten or will eat.

Glu-boroPro containing compounds may be administered together with other therapeutic agents, such as those discussed above. As used herein, a therapeutic agent is intended to embrace agents that work therapeutically and/or prophylactically. Depending on the timing and route of administration, the Glu-boroPro containing compound(s) and the second therapeutic agent(s) may be administered in the same administration vehicle (e.g., tablet, implant, injectable solution, etc.0, or alternatively,they may be separately dosed and administered.

Glu-boroPro containing compounds may be administered substantially simultaneously with the other therapeutic agents. By substantially simultaneously, it is meant that the Glu-boroPro containing compound is administered to a subject close enough in time with the administration of the other agent so that the compound and agent may exert an additive or even synergistic effect. The compounds of the invention may be administered or used together with non-drug therapies such as but not limited to non-drug anti-diabetic therapies such as carbohydrate reduced diets.

One therapeutic agent of interest is an anti-diabetic agent. An anti-diabetic agent is an agent that is used in the prevention and/or treatment of prediabetes or diabetes in order to regulate glucose. There are various categories of anti-diabetic agents and the varoius categories may have different mechanisma of cation. Anti-diabetic agents include insulin, peroxisome proliferator-activated receptor-γ (PPARγ) agonists, inhibitors of hepatic glucose production, stimulators of insulin release from pancreas, glucosidase inhibitors, and incretin and incretin analogues. Anti-diabetic agents include anti-hyperglycemia agents which are agents that lower blood sugar levels. Anti-hyperglycemia agents are known to those of ordinary skill in the art.

Insulin includes rapid-acting forms, intermediate-acting forms, and long-acting forms. Basal insulin, using long-acting insulins, can be injected once or twice a day. Bolus (or mealtime) insulin, using rapid-acting insulins, covers mealtime carbohydrates and corrects the current glucose level.

Rapid-acting forms of insulin include Insulin lispro rDNA origin: HUMALOG® (1.5 mL, 10 mL, Eli Lilly and Company, Indianapolis, Ind.), HUMALOG® Mix 75/25-Pen, Insulin Injection (Regular Insulin) form beef and pork (regular ILETIN® I, Eli Lilly], human: rDNA: HUMULIN® R (Eli Lilly), HUMULIN® 50/50, HUMULIN® 70/30, NOVOLIN® R (Novo Nordisk, New York, N.Y.), NOVOLIN® 70/30 Human Insulin, NOVOLIN® 70/30 PenFill, NOVOLIN® Innolet, Semisynthetic: VELOSULIN® Human (Novo Nordisk), rDNA Human, Buffered: VELOSULIN® BR, pork: regular Insulin (Novo Nordisk), purified pork: Pork Regular ILETIN® II (Eli Lilly), Regular Purified Pork Insulin (Novo Nordisk), and Regular (Concentrated) ILETIN® II U-500 (500 units/mL, Eli Lilly); NovoLog Mix 70/30.

Intermediate-acting forms of insulin include Insulin Zinc Suspension, beef and pork: LENTE® ILETIN® I (Eli Lilly), Human, rDNA: HUMULIN(® L (Eli Lilly), HUMULIN N, HUMULIN® N pen, NOVOLIN® L (Novo Nordisk), NOVOLIN N Human Insulin, NOVOLIN® N PenFill; purified pork: LENTE® ILETIN® II (Eli Lilly), Isophane Insulin Suspension (NPH): beef and pork: NPH ILETIN® I (Eli Lilly), Human, rDNA: HUMULIN® N (Eli Lilly), NOVOLIN® N (Novo Nordisk), purified pork: Pork NPH Iletin® II (Eli Lilly), NPH-N (Novo Nordisk).

Long-acting forms of insulin include Insulin zinc suspension, extended (ULTRALENTE®, Eli Lilly), human, rDNA: HUMULIN® U (Eli Lilly), Lantus Injection.

PPARγ agonists function as insulin-sensitizing agents that primarily enhance peripheral glucose utilization. PPARγ is a nuclear receptor that regulates transcription of insulin-responsive genes that in turn control glucose production, transport, and utilization and regulate fatty acid metabolism.

An example of PPARγ agonists is thiazolidinediones which include Avandamet (combination of rosiglitazone and metformin), rosiglitazone (Avandia), pioglitazone (Actos), troglitazone (Rezulin), (S)-((3,4-dihydro-2-(phenyl-methyl)-2H-1-benzopyran-6-yl)methyl-thiazolid-ine-2,4-dione (englitazone), 5- {[4-(3-(5-methyl-2-phenyl-4-oxazolyl)- 1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,-4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-y-1)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,-4-dione (rosiglitazone), 5-{[4-(2-(5-ethyl-2-pyridyl)ethoxy)phenyl]-methyl-}thiazolidine-2,4-dione (pioglitazone), 5-{[4-((3,4-dihydro-6-hydroxy-2,5,-7,8-tetramethyl-2H-1-benzopyran-2-yl)methoxy)-phenyl]-methyl}-thiazolidine-2,4-dione (troglitazone), 5-[6-(2-fluoro-benzyloxy)-naphthalen-2-ylmethyl-]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297).

Another example of a PPAR γ agonist is natural prostaglandin D(2) (PGD(2)) metabolite, 15-deoxy-Delta(12, 14)-prostaglandin J(2) (15d-PGJ(2)).

Other examples of PPAR γ agonists include GW-409544, GW-501516, and LY-510929.

Inhibitors of hepatic glucose production act primarily by decreasing hepatic glucose production, decreasing intestinal absorption of glucose and increasing peripheral glucose uptake and utilization. They can function as anti-hyperglycemic agents thereby lowering both basal and postprandial plasma glucose levels. An example of this category of agents is biguanides. Examples of biguanides include metformin (GLUCOPHAGE), Avandamet tablets (metformin combination tablet), Glucovance tablets, and Metaglip tablets.

Stimulators of insulin release from the pancreas act by a mechanism that is unclear, at least for long-term administration effect. When chronically administered, the blood glucose lowering effect of these agents persists despite a gradual decline in insulin secretory response. Extra-pancreatic effects may play a role in the mechanism of action. Examples of this category of agents are sulfonylureas and meglitinides. First-generation sulfonylureas include acetohexamide (DYMELOR), chlorpropamide (DIABINESE) and tolbutamide (ORINASE, RASTINON). Second-generation sulfonylureas include glipizide (GLUCOTROL, GLUCOTROL XL), glyburide (DIABETA; MICRONASE; GLYNASE) and glimepiride (AMARYL). Other sulfonylureas include glisoxepid (PRO-DIABAN), glibenclamide (AZUGLUCON), glibomuride (GLUBORID), tolazamide, carbutamide, gliquidone (GLURENORM), glyhexamide, phenbutamide, tolcyclamide, gliclazide (DIAMICRON).

Meglitinides close ATP-dependent K+channels in 0-cell membrane (selectively vs. heart and skeletal muscle), thereby depolarizing β-cells with consequent opening of Ca2+ channels. The resultant increased Ca²⁺ influx induces insulin secretion. Examples of meglitinides include Repaglinide (PRANDIN) and nateglinide (STARLIX).

Glucosidase inhibitors act by reversibly inhibiting membrane bound intestinal α-glucoside hydrolase enzymes. These enzymes hydrolyze oligosaccharides and disaccharides to glucose in the brush border of the small intestine. Pancreatic α-amylase, which hydrolyzes complex to oligosaccharides in lumen of small intestine, is also inhibited. The enzyme inhibition delays glucose absorption and lowers postprandial hyperglycemia. Examples of alpha-glucosidase inhibitors include Acarbose (PRECOSE, GLUCOBAY), Miglitol (GLYSET, DIASTABOL), and voglibose. Acarbose is 4″,6″-dideoxy-4″-[(1S)-(1,4,6/5)-4,5,6-trihydroxy-3-hydroxymethyl-2-cyclo- -hexenylamino}maltotriose (U.S. Pat. No. 4,062,950 and EP 0 226 121).

Incretins and incretin analogues can be used as anti-diabetic agents. These include GLP-1, GIP and their analogues. Analogues of glucagon like peptide-1 (GLP-1) include EXENATIDE (synthetic exendin-4) and EXENATIDE LAR (long acting release).

Other anti-diabetic agents include Buformin; Butoxamine Hydrochloride; Camiglibose; Ciglitazone; Englitazone Sodium; Darglitazone Sodium; Etoformin Hydrochloride; Gliamilide; Glicetanile Gliclazide Sodium; Gliflumide; Glucagon; Glymidine Sodium; Glyoctamide; Glyparamide; Linogliride; Linogliride Fumarate; Methyl Palmoxirate; Palmoxirate Sodium; Pirogliride Tartrate; Proinsulin Human; Seglitide Acetate; Tolpyrramide; Zopolrestat; and NVP-LAF237.

Further anti-diabetic agents are described in detail in U.S. Pat. Nos. 6,121,282, 6,057,343, 6,048,842, 6,037,359, 6,030,990, 5,990,139, 5,981,510, 5,980,902, 5,955,481, 5,929,055, 5,925,656, 5,925,647, 5,916,555, 5,900,240, 5,885,980, 5,849,989, 5,837,255, 5,830,873, 5,830,434, 5,817,634, 5,783,556, 5,756,513, 5,753,790, 5,747,527, 5,731,292, 5,728,720, 5,708,012, 5,691,386, 5,681,958, 5,677,342, 5,674,900, 5,545,672, 5,532,256, 5,531,991, 5,510,360, 5,480,896, 5,468,762, 5,444,086, 5,424,406, 5,420,146, RE34,878, 5,294,708, 5,268,373, 5,258,382, 5,019,580, 4,968,707, 4,845,231, 4,845,094, 4,816,484, 4,812,471, 4,740,521, 4,716,163, 4,695,634, 4,681,898, 4,622,406, 4,499,279, 4,467,681, 4,448,971, 4,430,337, 4,421,752, 4,419,353, 4,405,625, 4,374,148, 4,336,391, 4,336,379, 4,305,955, 4,262,018, 4,220,650, 4,207,330, 4,195,094, 4,172,835, 4,164,573, 4,163,745, 4,141,898, 4,129,567, 4,093,616, 4,073,910, 4,052,507, 4,044,015, 4,042,583, 4,008,245, 3,992,388, 3,987,172, 3,961,065, 3,954,784, 3,950,518, 3,933,830, the disclosures of which are incorporated herein by reference.

The invention also contemplates the use of a second agent that is also a DPP-IV inhibitor. These include but are not limited to alanyl pyrrolidine, isoleucyl thiazolidine and O-benzoyl hydroxylamine.

Anti-diabetic agents also include combinations of anti-diabetic agents, many of which are commercially available. These include ACTOS(R) (pioglitazone HCl) in combination with a sulfonylurea, metformin or insulin.

Table 4 shows a list of anti-diabetic agents used singly or in combination.

TABLE 4
Anti-diabetic drug categories

| | Proprietary drug | |
|---|---|---|
| Category | trade name | Anti-diabetic agents in drug |
| Biguanides and | Avandamet | Rosiglitazone maleate |
| combinations | | (thiazolidinedione) + |
| | | metformin HCl (biguanide) |
| | Glucovance | Glyburide (sulphonylurea) + |
| | | metformin HCl (biguanide) |
| | Metaglip | Glipizide (sulphonylureas) + |
| | | metformin HCl (biguanide) |
| Glucosidase | Glyset | Miglitol (oral α-glucosidase |
| inhibitors | | inhibitor) |
| | Precose | Ascarbose (oral α-glucosidase |
| | | inhibitor) |
| Meglitinides | Prandin | Repaglinide (oral meglitinide) |
| | Starlix | Nateglinide (oral meglitinide) |
| Sulfonylurea | Amaryl | Glimepiride (oral |
| | | sulfonylurea) |
| | Diaβeta | Glyburide (oral sulfonylurea) |
| | Diabinese | Chlorpropamide (oral |
| | | sulfonylurea) |
| | Glucotrol | Glipizide (oral sulfonylurea) |
| Thiazolidinediones | Actos | Pioglitazone HCl (oral |
| | | thiazolidinedione) |
| | Avandia | Rosiglitazone maleate (oral |
| | | thiazolidinedione) |

Anti-inflammatory agents are agents that reduce inflammation locally or systemically in a subject. Examples of anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Examples of second agents that are generally useful in this invention include but are not limited to anti-diabetic agents, anti-obesity agents, anti-atherosclerotic agents, anti-retinopathy agents, anti-hyperlipidemia agents, anti-acidosis agents, anti-neuropathy agents, anti-nephropathy agents, anti-hyperglycemia agents, anti-hyperinsulinemia agents, anti-hyperlipoproteinemia agents, anti-hypertension agents, anti-inflammatory agents, anti-ulcer agents, and the like. Those of ordinary skill in the art will be familiar with such agents, and in addition reference can be made to Harrison's Principles of Internal Medicine, 15^{th} Edition, McGraw-Hill, Inc., N.Y. or the Physician's Desk Reference (PDR).

Other therapeutic agents of interest that may be administered with Glu-boroPro include: anti-thrombotic agents, fibrinolytic agents, anti-platelet agents, direct thrombin inhibitors, glycoprotein II b/IIIa receptor inhibitors, agents that bind to cellular adhesion molecules and inhibit the ability of white blood cells to attach to such molecules (e.g. anti-cellular adhesion molecule antibodies), alpha-adrenergic blockers, beta-adrenergic blockers, cyclooxygenase-2 inhibitors, angiotensin system inhibitor, anti-arrhythmics, calcium channel blockers, diuretics, inotropic agents, vasodilators, vasopressors, and/or any combinations thereof.

Anti-hyperlipidemia agents are agents that reduce total cholesterol, LDLC, or triglycerides, or that increase HDLC. Anti-hyperlipidemia agents include statins and non-statin anti-hyperlipidemia agents, and/or combinations thereof. Anti-hyperlipidemia and anti-hyperlipoproteinemia are used interchangeably herein. Statins (also called HMG-CoA reductase inhibitors) are a class of medications that have been shown to be effective in lowering human total cholesterol, LDLC and triglyceride levels. Statins act at the step of cholesterol synthesis. By reducing the amount of cholesterol synthesized by the cell, through inhibition of the HMG-CoA reductase gene, statins initiate a cycle of events that culminates in the increase of LDLC uptake by liver cells. As LDLC uptake is increased, total cholesterol and LDLC levels in the blood decrease. Lower blood levels of both factors are associated with lower risk of atherosclerosis and heart disease, and the statins are widely used to reduce atherosclerotic morbidity and mortality.

Examples of statins include, but are not limited to, simvastatin (Zocor) (U.S. Pat. No. 4,444,784), lovastatin (Mevacor) (U.S. Pat. No. 4,231,938), pravastatin (Pravachol) (U.S. Pat. No. 4,346,227), fluvastatin (Lescol) (U.S. Pat. No. 4,739,073), atorvastatin (Lipitor) (U.S. Pat. No. 5,273,995), cerivastatin (Baycol), rosuvastatin (Crestor), pitivastatin and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402. Another statin is compound 3a (S-4522) described in Watanabe (1997) Bioorganic and Medicinal Chemistry 5:437-44. Examples of statins already approved for use in humans include atorvastatin, cerivastatin, fluvastatin, pravastatin, simvastatin and rosuvastatin.

Further information on HMG-CoA reductase inhibitors can be found in Drugs and Therapy Perspectives (May 12, 1997), 9: 1-6; Chong (1997) Pharmacotherapy 17:1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368.

Non-statin anti-hyperlipidemia agents include but are not limited to fibric acid derivatives (i.e., fibrates), bile acid sequestrants or resins, nicotinic acid agents, cholesterol absorption inhibitors, acyl-coenzyme A: cholesterol acyl transferase (ACAT) inhibitors, cholesteryl ester transfer protein (CETP) inhibitors, LDL receptor antagonists, farnesoid X receptor (FXR) antagonists, sterol regulatory binding protein cleavage activating protein (SCAP) activators, microsomal triglyceride transfer protein (MTP) inhibitors, squalene synthase inhibitors, and peroxisome proliferation activated receptor (PPAR) agonists.

Examples of fibric acid derivatives include but are not limited to gemfibrozil (Lopid), fenofibrate (Tricor), clofibrate (Atromid), and bezafibrate.

Examples of bile acid sequestrants or resins include but are not limited to colesevelam (WelChol), cholestyramine (Questran or Prevalite) and colestipol (Colestid), DMD-504, GT-102279, HBS-107, and S-8921.

Examples of nicotinic acid agents include but are not limited to niacin and probucol.

Examples of cholesterol absorption inhibitors include but are not limited to ezetimibe (Zetia).

Examples of ACAT inhibitors include but are not limited to Avasimibe, CI-976 (Parke Davis), CP- 113818 (Pfizer), PD-138142-15 (Parke Davis), F1394, and numerous others described in U.S. Pat. Nos. 6,204,278, 6,165,984, 6,127,403, 6,063,806, 6,040,339, 5,880,147, 5,621,010, 5,597,835, 5,576,335, 5,321,031, 5,238,935, 5,180,717, 5,149,709, and 5,124,337.

Examples of CETP inhibitors include but are not limited to Torcetrapib, CP-529414, CETi-1, JTT-705, and numerous others described in U.S. Pat. Nos. 6,727,277, 6,723,753, 6,723,752, 6,710,089, 6,699,898, 6,696,472, 6,696,435, 6,683,099, 6,677,382, 6,677,380, 6,677,379, 6,677,375, 6,677,353, 6,677,341, 6,605,624, 6,586,448, 6,521,607, 6,482,862, 6,479,552, 6,476,075, 6,476,057, 6,462,092, 6,458,852, 6,458,851, 6,458,850, 6,458,849, 6,458,803, 6,455,519, 6,451,830, 6,451,823, 6,448,295, and 5,512,548.

One example of an FXR antagonist is Guggulsterone. One example of a SCAP activator is GW532 (GlaxoSmithKline).

Examples of MTP inhibitors include but are not limited to Implitapide and R-103757.

Examples of squalene synthase inhibitors include but are not limited to zaragozic acids.

Anti-thrombotic agents and/or fibrinolytic agents include Plasminogen (to plasmin via interactions of prekallikrein, kininogens, Factors XII, XIIIa, plasminogen proactivator, and tissue plasminogen activator TPA]) Streptokinase, Urokinase: Anisoylated Plasminogen-Streptokinase Activator Complex, Pro-Urokinase, (Pro-UK), rTPA (alteplase or activase, r denotes recombinant), rPro-UK, Abbokinase, Eminase, Sreptase Anagrelide Hydrochloride, Bivalirudin, Dalteparin Sodium, Danaparoid Sodium, Dazoxiben Hydrochloride, Efegatran Sulfate, Enoxaparin Sodium, Ifetroban, Ifetroban Sodium, Tinzaparin Sodium, retaplase, Trifenagrel, Warfarin, and Dextrans.

Anti-platelet agents include Clopridogrel, Sulfinpyrazone, Aspirin, Dipyridamole, Clofibrate, Pyridinol Carbamate, PGE, Glucagon, Antiserotonin drugs, Caffeine, Theophyllin Pentoxifyllin, Ticlopidine, and Anagrelide.

Direct thrombin inhibitors include hirudin, hirugen, hirulog, agatroban, PPACK, and thrombin aptamers.

Glycoprotein Ilb/IIIa receptor Inhibitors are both antibodies and non-antibodies, and include but are not limited to ReoPro (abcixamab), lamifiban, and tirofiban.

Agents that bind to cellular adhesion molecules and inhibit the ability of white blood cells to attach to such molecules include polypeptide agents. Such polypeptides include polyclonal and monoclonal antibodies, prepared according to conventional methodology. Such antibodies already are known in the art and include anti-ICAM 1 antibodies as well as other such antibodies.

Thus, the invention encompasses polypeptides of numerous size and type that bind specifically to cellular adhesion molecules. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Examples of alpha-adrenergic blockers include doxazocin, prazocin, tamsulosin, and tarazosin.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, beftnolol, betaxolol, bunitrolol, carteolol, celiprolol, hedroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, and 7-(2-hydroxy-3-t-butylaminpropoxy)phthalide. The above-identified compounds can be used as isomeric mixtures, or in their respective levorotating or dextrorotating form.

Cyclooxygenase-2 (COX-2) is a recently identified new form of a cyclooxygenase. Cyclooxygenase is an enzyme complex present in most tissues that produces various prostaglandins and thromboxanes from arachidonic acid. Non-steroidal, antiinflammatory drugs exert most of their antiinflammatory, analgesic and antipyretic activity and inhibit hormone-induced uterine contractions and certain types of cancer growth through inhibition of the cyclooxygenase (also known as prostaglandin G/H synthase and/or prostaglandin-endoperoxide synthase). Initially, only one form of cyclooxygenase was known, the “constitutive enzyme” or cyclooxygenase-1 (COX-1). It was originally identified in bovine seminal vesicles.

Cyclooxygenase-2 (COX-2) has been cloned, sequenced and characterized initially from chicken, murine and human sources (See, e.g., U.S. Pat. No 5,543,297, issued Aug. 6, 1996 to Cromlish, et al., and assigned to Merck Frosst Canada, Inc., Kirkland, Calif., entitled: “Human cyclooxygenase-2 cDNA and assays for evaluating cyclooxygenase-2 activity”). This enzyme is distinct from the COX-1. COX-2, is rapidly and readily inducible by a number of agents including mitogens, endotoxin, hormones, cytokines and growth factors. As prostaglandins have both physiological and pathological roles, it is believed that the constitutive enzyme, COX-1, is responsible, in large part, for endogenous basal release of prostaglandins and hence is important in their physiological fimctions such as the maintenance of gastrointestinal integrity and renal blood flow. By contrast, it is believed that the inducible form, COX-2, is mainly responsible for the pathological effects of prostaglandins where rapid induction of the enzyme would occur in response to such agents as inflammatory agents, hormones, growth factors, and cytokines. Therefore, it is believed that a selective inhibitor of COX-2 has similar antiinflammatory, antipyretic and analgesic properties to a conventional non-steroidal antiinflammatory drug, and in addition inhibits hormone-induced uterine contractions and also has potential anti-cancer effects, but with reduced side effects. In particular, such COX-2 inhibitors are believed to have a reduced potential for gastrointestinal toxicity, a reduced potential for renal side effects, a reduced effect on bleeding times and possibly a decreased potential to induce asthma attacks in aspirin-sensitive asthmatic subjects, and are therefore useful according to the present invention.

A number of selective COX-2 inhibitors are known in the art. These include, but are not limited to, COX-2 inhibitors described in U.S. Pat No. 5,474,995 “Phenyl heterocycles as COX-2 inhibitors”; U.S. Pat No. 5,521,213 “Diaryl bicyclic heterocycles as inhibitors of cyclooxygenase-2”; U.S. Pat No. 5,536,752 “Phenyl heterocycles as COX-2 inhibitors”; U.S. Pat. No. 5,550,142 “Phenyl heterocycles as COX-2 inhibitors”; U.S. Pat No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat No. 5,639,780 N-benzyl indol-3-yl butanoic acid derivatives as cyclooxygenase inhibitors”; U.S. Pat No. 5,677,318 Diphenyl-1,2-3-thiadiazoles as anti-inflammatory agents”; U.S. Pat No. 5,691,374 “Diaryl-5-oxygenated-2-(5H)-furanones as COX-2 inhibitors”; U.S. Pat. No. 5,698,584 “3,4-diaryl-2-hydroxy-2,5-dihydrofurans as prodrugs to COX-2 inhibitors”; U.S. Pat No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin I and angiotensin II are synthesized by the enzymatic renin-angiotensin pathway. The synthetic process is initiated when the enzyme renin acts on angiotensinogen, a pseudoglobulin in blood plasma, to produce the decapeptide angiotensin I. Angiotensin I is converted by angiotensin converting enzyme (ACE) to angiotensin II (angiotensin-[1-8] octapeptide). The latter is an active pressor substance which has been implicated as a causative agent in several forms of hypertension in various mammalian species, e.g., humans.

Angiotensin (renin-angiotensin) system inhibitors are compounds that act to interfere with the production of angiotensin II from angiotensinogen or angiotensin I or interfere with the activity of angiotensin II. Such inhibitors are known to those of ordinary skill in the art and include compounds that act to inhibit the enzymes involved in the ultimate production of angiotensin II, including renin and ACE. They also include compounds that interfere with the activity of angiotensin II, once produced. Examples of classes of such compounds include, but are not limited to, antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists. In a preferred embodiment of the invention, the renin-angiotensin system inhibitors are renin inhibitors, ACE inhibitors, and angiotensin II antagonists.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include, but are not limited to, peptidic compounds (e.g., saralasin, [(San¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1 -(2-chlorobenzile) imidazole-5-acetic acid (see Long et al., J. Pharmacol. Exp. Ther. 247(1), 1-7 (1988)); 4, 5, 6, 7-tetrahydro-1H-imidazo [4, 5-c] pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1, 3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl] 1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffinan LaRoche AG); A₂ agonists (Marion Merrill Dow) and certain non-peptide heterocycles (G.D.Searle and Company).

Angiotensin converting enzyme (ACE), is an enzyme which catalyzes the conversion of angiotensin I to angiotensin II. ACE inhibitors include amino acids and derivatives thereof, peptides, including di and tri peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents include urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos 5,071,837, 5,064,965, 5,063,207, 5,036,054, 5,036,053, 5,034,512, and 4,894,437).

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogenous group of drugs that prevent or slow the entry of calcium into cells by regulating cellular calcium channels. (Remington, The Science and Practice ofpharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11-2933), phenytoin, barbiturates, and the peptides dynorphin, omega-conotoxin, and omega-agatoxin, and the like and/or pharmaceutically acceptable salts thereof.

Diuretics include, but are not limited to, carbonic anhydrase inhibitors, loop diuretics, potassium-sparing diuretics, thiazides and related diuretics.

Vasodilators include, but are not limited to, coronary vasodilators and peripheral vasodilators.

Anti-obesity agents include, but are not limited to, catecholamines, sympathomimetics and lipase inhibitors. Examples of anti-obesity agents include amphetamine, metamphetamine, metamphetamine HCL (Desoxyn), phentermine, phentermine HCL (Adipex), phentermine resin (lonamin), benzphetamine, phendimetrazine, phenmetrazine, diethylpropion, mazindol, fenfluramine, phenylpropanolamine, sibutramine, sibutramine HCL monohydrate (Meridia), and Orlistat (Xenical).

Anti-retinopathy agents are agents used to treat retinopathy. Such agents are known to those of ordinary skill in the art.

Anti-neuropathy agents include, but are not limited to, acetaminophen, non-steroidal anti-inflammatory drugs (e.g., ibuprofen and naprosyn), antidepressants (e.g., amitriptyline (Elavil), and desipramine (Norpranin)), anticonvulsants (e.g., carbamazepine (Tegretol), phenytoin (Dilantin), and gabapentin (Neurontin)), baclofen (lioseral), mexiletine (Mexitil), prazocin (Minipress), and narcotics (e.g., hydrocodone, codeine and morphine).

Anti-nephropathy agents include, but are not limited to, angiotensin converting enzyme inhibitors (ACE inhibitors), angiotensin type II receptor blockers (ARBs), combinations of ACE inhibitors and ARBs, combination of ACE inhibitors and indapamide, combination of ACE inhibitors and aldosterone antagonists (e.g., spironolactone), combination of ACE inhibitors and endopeptidase inhibitor (e.g., omapatrilat).

Anti-acidosis agents are agents that correct acidosis in the blood and include, for example, bicarbonates. Anti-acidosis agents are known to those of ordinary skill in the art.

A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular or infusion. Intravenous or intramuscular routes are not particularly suitable for long-term therapy and prophylaxis. They could, however, be preferred in emergency situations. Oral administration is a generally preferred mode of administration because of the convenience to the patient.

Encompassed by the invention are compositions comprising a Glu-boroPro containing compound(s) and a second agent. Also encompassed by the invention are pharmaceutical compositions comprising a Glu-boroPro containing compound(s) and a second agent as described below.

When used in vivo, the agents are formulated as pharmaceutical compositions or preparations. A pharmaceutical preparation is a composition suitable for administration to a subject. Such preparations are usually sterile and prepared according to GMP standards, particularly if they are to be used in human subjects. In general, a pharmaceutical composition or preparation comprises the agent(s) and a pharmaceutically-acceptable carrier, wherein the agent(s) are generally provided in effective amounts As used herein, a pharmaceutically-acceptable carrier means a non-toxic material that does not interfere with the effectiveness of the biological activity of the agents of the invention.

Pharmaceutically-acceptable carriers include diluents, fillers, salts, buffers, stabilizers, solubilizers and other materials which are well-known in the art. Exemplary pharmaceutically-acceptable carriers for peptides in particular are described in U.S. Pat. No. 5,211,657. Such preparations may routinely contain salt, buffering agents, preservatives, compatible carriers, and optionally other therapeutic or prophylactic agents. When used in medicine, the salts should be pharmaceutically acceptable, but non-pharmaceutically-acceptable salts may conveniently be used to prepare pharmaceutically-acceptable salts thereof and are not excluded from the scope of the invention. Such pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compositions of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Preferably, at least the Glu-boroPro containing compounds are formulated for oral administration. Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

For oral administration, the agents can be formulated readily by combining the active compound(s) with pharmaceutically-acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

The compsitions may be administered directly to a tissue. Preferably, the tissue is one that is likely to respond beneficially to the compositions. Direct tissue administration may be achieved by direct injection. If the agents are administered multiple times, the compositions may be administered via different routes. For example, the first (or the first few) administrations may be made directly into the affected tissue while later administrations may be systemic.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Other delivery systems can include time-release, delayed release or sustained release delivery systems. Such systems can avoid repeated administrations of the agent, increasing convenience to the subject and the physician. Many types of release delivery systems are available and known to those of ordinary skill in the art. They include polymer based systems and non-polymer based systems such as lipids including sterols such as cholesterol, cholesterol esters and fatty acids or neutral fats such as mono-, di-, and tri-glycerides; hydrogel release systems; silastic systems; peptide based systems; wax coatings; compressed tablets using conventional binders and excipients; partially fuised implants; and the like. Specific examples include, but are not limited to: (a) erosional systems in which the agent is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for prophylactic treatment of subjects. Long-term release, as used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments the delivery vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used. The polymeric matrix composition can be selected to have both favorable degradation rates and also to be formed of a material which is bioadhesive, to fiuther increase the effectiveness of transfer. The matrix composition also can be selected not to degrade, but rather, to release by diffusion over an extended period of time.

Both non-biodegradable and biodegradable polymeric matrices can be used to deliver the compounds and/or agents to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the compounds and/or agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polymers of lactic acid and glycolic acid, polyanhydrides, poly(ortho)esters, polyurethanes, poly(butic acid), poly(valeric acid), and poly(lactide-cocaprolactone) and polyvinylpyrrolidone.

Examples of biodegradable polymers include natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The invention further provides kits that comprise the compounds and/or agents of the invention and optionally instructions of use thereof. The compounds and/or agents may be present in oral forms such as tablets, pills, capsules, caplets and the like. The compounds and/or agents may be provided in a one a day dispensing unit such as a blister pack or dial pack type dispenser, preferably with days of the week or day of the month (e.g., 1, 2, 3, 4, etc.) (and doses per day, where applicable) printed on the dispenser. For example, if the compounds and/or agents are to be administered every other day or twice (or more) a day, the dispensing unit can be modified accordingly, with no more than routine reconfiguration, known in the art. The kit may further contain a second agent such as a second anti-diabetic agent, either formulated together with the Glu-boroPro containing compound of the invention or formulated separately. The unit dosages provided in each form (e.g., tablet, pill, capsule, etc.) will depend upon whether the Glu-boroPro containing compound is used together with or in the absence of a second agent. The kit may optionally comprise a housing such as a box or bag. Instructions for use may be supplied separately from the dispensing unit or housing or they may be imprinted on one or both.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

This example illustrates the kinetics of in vitro DPP-IV inhibition by Glu-boroPro. The enzyme inhibitory activity of Glu-boroPro is compared with that of other amino boronic dipeptides in in vitro assays with isolated DPP-IV.

Materials and Methods

Production of soluble recombinant human DPP-IV. Based on information on the N-terminus of serum DPP-IV (15), a truncated DPP-IV was engineered in which a signal/leader sequence was joined to the residue in DPP-IV corresponding to the N-terminus of serum DPP-IV to allow secretion. The cDNA encoding the desired truncated human DPP-IV dimer enzyme was engineered into the mammalian secretion vector pSecTag2 (Cat# V900-20, InVitrogen Corporation). The vector, available in A, B or C versions, representing three possible phases for gene fusion, contained an immunoglobulin-kappa light chain secretion signal followed by a selection of restriction sites for gene insertion. The fusion required engineering a restriction site upstream of the chosen fusion amino acid in the 5′ end of the DPP-IV dimer enzyme nucleic acid in phase with the chosen restriction site (Sfi I) in the vector secretion sequence. The chosen fusion amino acid in the 5′ end of the DPP-IV (Ser39) was 3′ of the trans-membrane anchoring domain. The pSecTag2 version B and its Sfi I restriction site was chosen for the fusion because it minimizes the additional N-terminal, vector-encoded residues in the mature secreted protein.

Sequence of N-terminus of Serum hDPP-IV (15)

| | | |
|---|---|---|
| hDPP-IV: | MKTPWKVLLGLLGAAALVTIITVPVVLLNKGTDDATADSRKTYTLTDYLKN-- | (SEQ ID NO: 1) |
| Serum DPP-IV: | SRKTYTLTDYLKN-- | (SEQ ID NO: 2) |
| | RKTYTLTDYLKN-- | (SEQ ID NO: 3) |

Construction of the fusion was as follows. First, total RNA was isolated from the Caco-2 colorectal carcinoma cell line (ATCC HTB-37) by standard Trizol/phenol/chloroform methodology. The purified RNA (approx. 2.5 μg in a 20 μl reaction) was used to make cDNA using oligo-dT primer and a commercial reverse transcription (RT) kit (InVitrogen). An aliquot (2 μl) of the RT reaction was used to amplify by polymerase chain reaction (PCR), a truncated coding region of human DPP-IV dimer enzyme cDNA corresponding to nucleotide 225-2408 approximately of wild type DPP-IV dimer enzyme (GenBank Accession number NM_{—}001935). The Taq DNA polymerase-mediated PCR was performed with primers: Sfi-DPP-IV (5′ GTAGTCGGCC CAGCCGGCC AGTCGCAAAA CTTACACTCT AACTGATTAC TTAAAAAAT 3′, SEQ ID NO: 4) containing a Sfi I restriction site (underlined) and primer DPP4-R 5′ GTCGGAGCGG CCGCCTAAGG TAAAGAGAAA CATTGTTTTA TGAAGTG 3′ (SEQ ID NO: 5) containing a Not I site. The following thermal cycler program was used: 94° C. for 45 sec. initial denaturation, then 30 cycles of 94° C., 10 sec.; 48° C., 6 sec.; 60° C., 4 min; followed by 5-min. extension at 72° C. after cycling. The resultant PCR product was cleaved with restriction enzymes SfiI for 25 min at 50° C., then 1 hr with NotI at 37° C. The approx. 2.2 kb fragment was isolated from an agarose gel using standard procedures and ligated to pSecTag2-B vector (InVitrogen, Cat. # V900-20) fragments (5.6 kb) that had been similarly prepared using the same enzymes. After transformation into bacteria under standard conditions and screening of colonies, those with correct properties were sequenced to ensure the correct fusion junction and absence of PCR-induced mutations, giving a plasmid designated #135 which was designated wild-type DPP-IV. The resulting plasmid #135, contained DPP-IV truncated, as described above, and fused to a plasmid encoded immunoglobulin Kappa chain secretion sequence under control of the CMV promoter (U.S. Pat. Nos. 5,168,062 and 5,383,839) with a 3′ bovine growth hormone polyadenylation sequence (U.S. Pat. No. 5,122,458). The N-terminus of the final mature amino acid sequence of mature (cleaved) secreted product contains 6 amino acids having a sequence of DAAQPR (SEQ ID NO:6) or DAAQPA (SEQ ID NO:7), fused to the truncated DPP-IV sequence starting at Ser39, the first 13 amino acids of which are SRKTYTLTDYLKN (SEQ ID NO:2).

DNA from the plasmid encoding DPP-IV dimer enzyme was prepared on an approximately 400 μg scale from overnight 30 ml cultures in Luria broth with 100 μg ampicillin per ml using a commercial kit (Qiagen Maxiprep Kit). Ten (10) μg of DNA and 30 μl of Lipofectamine 2000 transfection reagent (InVitrogen Corporation) were used to transiently transfect 293T cells in 10 cm diameter tissue culture plates using the manufacturer's protocol. Cells were grown to >70% confluent in Freestyle 293 Expression Medium (InVitrogen Corporation) containing 2.5% fetal calf serum and standard antibiotics penicillin and streptomycin. Antibiotic-free medium was used for the initial 18-24 hours of transfection, after which serum-free medium with antibiotics was employed. Culture supernatant containing the secreted recombinant enzyme was harvested 6-18 hours later and again 24 hours after addition of fresh serum-free medium and was stored 4° C.

In vitro assay of enzmatic activity ofrecombinant soluble DPP-IV and inhibition by amino boronic dipeptides. The assay reaction mixture consisted of 135 μl 50 mM HEPES/Na buffer pH 7.6, 140 mM NaCl, 10-15 gl enzyme-containing culture supernatant, dipeptide substrate Ala-Pro-(7-amino-4-trifluoromethyl coumarin) (Ala-Pro-AFC; Enzyme System Products, Dublin, Calif.) at typically 0.1-1 mM added from a 100 or 400 mM stock in dimethyl formamide. The reaction mixture was incubated at room temperature, and production of the fluorescent AFC product was measured in a fluorometer (excitation, 400 nm; emission 505 nm), either by continuous monitoring or after termination with a one to one-tenth volume of 1 -M sodium acetate, pH 4.5. Fluorometric reading were made with a Molecular Dynamics Spectra Max GeminiXS capable of reading 96-well microtiter plates. The inhibitory activity of amino boronic dipeptides was investigated by preincubation of assay reaction mixtures with varying concentrations of each compound for 10 minutes before the addition of the substrate Ala-Pro-AFC. The completed reaction mixtures were then incubated for 3 minutes, 10 minutes, 78 minutes, or 16 hours and read fluorometrically.

Results

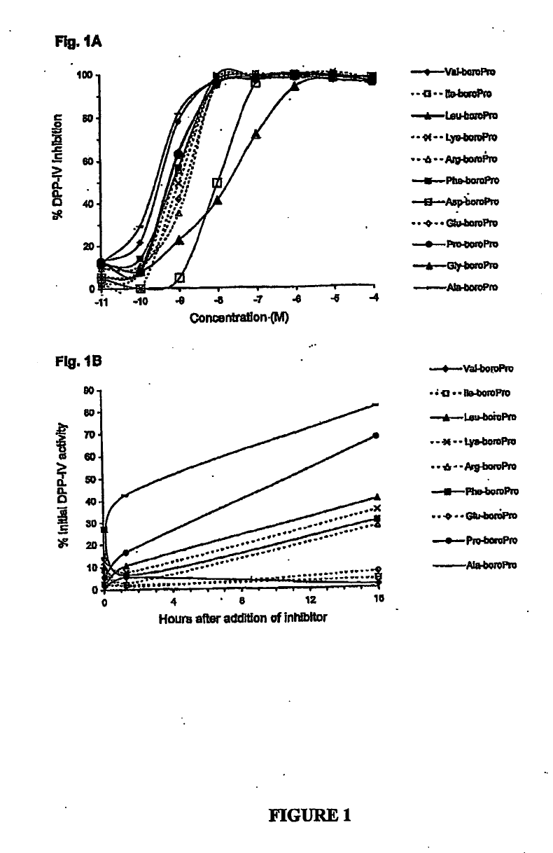

FIG. 1A illustrates an in vitro dose-response comparison of soluble recombinant DPP-IV enzymatic inhibition by Val-boroPro, Ile-boroPro, Leu-boroPro, Lys-boroPro, Arg-boroPro, Phe-boroPro, Asp-boroPro, Glu-boroPro, Pro-boroPro, Gly-boroPro, and Ala-boroPro. All the amino boronic dipeptides except Asp-boroPro and Gly-boroPro exhibited IC₅ (inhibitory concentration 50%, i.e., the concentration of compound required to inhibit enzymatic activity by 50% of control activity) values in the low to sub nanomolar range when DPP-IV was preincubated for 10 minutes with each amino boronic dipeptide before addition of the substrate, Ala-Pro-AFC, and fluorometric measurement after further incubation for 10 minutes. In a separate experiment, comparison of DPP-IV inhibition assayed at 3 minutes, 78 minutes and 16 hours after the addition of Ala-Pro-AFC revealed that preincubation with Glu-boroPro, Val-boroPro and Ile-boroPro resulted in sustained DPP-IV inhibition (>10% of initial DPP-IV activity) for up to 16 hours, whereas inhibition by Ala-boroPro, Pro-boroPro, Leu-boroPro, Lys-boroPro, Phe-boroPro, and Arg-boroPro appeared to be more rapidly reversible (FIG. 1 B).

### Example 2

This example illustrates the kinetics of serum DPP-IV inhibition by Glu-boroPro in vivo in mice. The enzyme inhibitory activity of Glu-boroPro is compared with that of other amino boronic dipeptides in in vitro assays with isolated DPP-IV.

Materials and Methods

Assay of serum DPP-IV inhibition in vivo. Varying doses (0.02, 0.2, 2.0, 20.0 μg/mouse) of Glu-boroPro dissolved in normal saline or the saline vehicle alone were administered to BALB/c mice by oral gavage. Each mouse received a single administration of Glu-boroPro or saline, and blood samples were withdrawn from mice 2 hours later. In studies of the duration of DPP-IV inhibition after administration of 5 or 10 μg/mouse of Glu-boroPro, blood samples were withdrawn at 1, 2, 4, 6, 11, 24, 26 and 48 hours after Glu-boroPro or saline administration. DPP-IV activity was determined by reaction of 10 μl serum with 90 μl of 0.11 mM Ala-Pro-AFC (Enzyme System Products, Dublin, Calif.) in 50 mM HEPES/Na buffer pH 7.6, 140 mM NaCl. Assays were incubated for 30 minutes, stopped and fluorometric measurements made as described in Example 1. Serum DPP-IV activity was expressed as a percentage of the baseline activity in control mice receiving saline, or the activity in mice prior to administration of Glu-boroPro.

Results

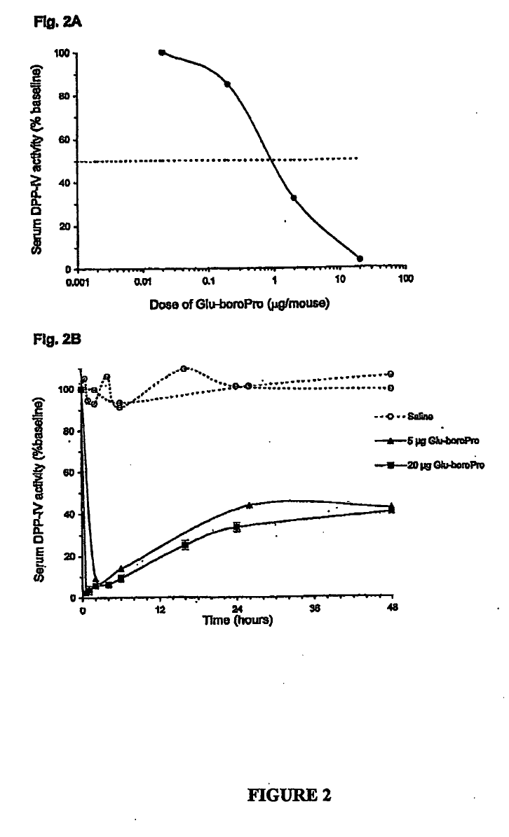

FIG. 2A illustrates a typical dose response for the inhibition of DPP-IV activity in the serum of BALB/c mice administered Glu-boroPro orally. In this experiment, the ID₅₀ (inhibitory dose 50%, i.e., the dose required to reduce serum DPP-IV activity by 50% of baseline in control animals) was determined to be a 1 μg dose of Glu-boroPro per mouse. The duration of serum DPP-IV inhibition after a single oral administration of 5 μg or 20 μg Glu-boroPro per mouse was determined in two experiments (FIG. 2B). The data indicate that greater than 80% of DPP-IV inhibition persisted until at least 6 hours after Glu-boroPro administration.

### Example 3

This example illustrates that, unlike the amino boronic peptides Val-boroPro, Ile-boroPro and Leu-boroPro, Glu-boroPro does not appear to stimulate cytokine production by cultured human bone marrow stromal cells in vitro, as indicated by measurement of the levels of granulocyte colony stimulating factor (G-CSF) in culture supernatants. G-CSF was assayed because it was previously shown to be an indicator of increased levels of cytokines in stromal cell cultures stimulated with Val-boroPro (16).

Materials and Methods

Human bone marrow stromal cell cultures. Samples of normal human bone marrow were purchased from Cambrex Bioproducts (Walkersville, Md.) and mononuclear cells were purified over Ficoll-Hypaque (Nycomed, Oslo, Norway). Human stromal layers were established by seeding 4×10⁷ mononuclear cells into T75 flasks (Corning) containing 20 ml MyeloCult medium (Stem Cell Technologies, Vancouver, BC) supplemented with 10⁻⁶ M hydrocortisone (Sigma) and incubation at 37° C. in 100% humidified 5% CO₂ in air. After one week, half the medium was exchanged, and the cultures incubated for approximately one week more, after which time, a semi-confluent cell layer was formed. Stromal cells were harvested by trypsinization using standard technique and 10⁵ cells/well were seeded in multi-well plates in 1 ml of fully supplemented DMEM (InVitrogen, Carlsbad, Calif.). Val-boroPro, Ile-boroPro, Leu-boroPro or Glu-boroPro were each added to triplicate multiwell cultures at concentrations of 1, 10, 100, 10³ and 10⁴ nM. Multiwell cultures without the addition of amino boronic dipeptides served as controls.

Assay of G-CSF supernatant levels in stromal cell cultures. After incubation of multi-well cultures for 24 hours, supernatants were harvested. Supernatant concentrations of human G-CSF were determined by Quantikine enzyme-linked immunosorbent assay (ELISA; R&D Systems, Minneapolis, Minn.) according to the manufacturer's instructions. ELISA was performed in duplicate for each sample. G-CSF concentrations were compared between cultures containing amino boronic dipeptide and control cultures. The effect of each amino boronic dipeptide on the level of supernatant G-CSF was determined by calculating a stimulation index (SI): SI=(mean G-CSF concentration in culture with amino boronic dipeptide)/(mean G-CSF concentration in control culture).

Results

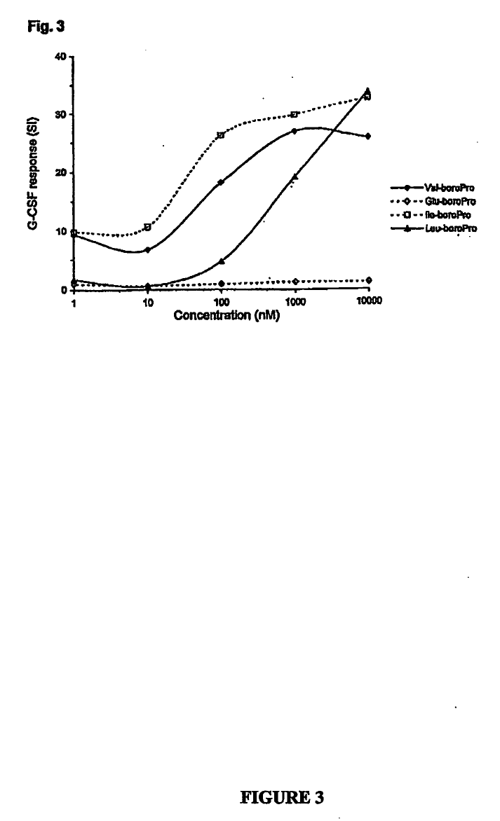

FIG. 3 illustrates the in vitro dose responses of human bone marrow stromal cell cultures to the addition of Val-boroPro, Ile-boroPro, Leu-boroPro or Glu-boroPro as determined by supernatant levels of G-CSF. The SI revealed that, unlike Val-boroPro, Ile-boroPro and Leu-boroPro, Glu-boroPro did not appear to stimulate increased levels of G-CSF in culture supernatants after incubation in vitro for 24 hours.

### Example 4

This example illustrates that, unlike the amino boronic peptides Val-boroPro, Ile-boroPro and Leu-boroPro, Glu-boroPro does not appear to stimulate increased levels of serum KC/CXCL1 in BALB/c mice in vivo at doses that optimally inhibit serum DPP-IV activity. Serum KC/CXCL 1 was assayed because it was previously shown to be an indicator of increased levels of cytokines and chemokines in the serum of mice administered Val-boroPro (16, 17).

Materials and Methods

Assay ofserum DPP-IV inhibition and KC/CXCL1 levels in vivo. Varying doses (0.2, 2.0, 20.0 and 200.0 μg/mouse) of Val-boroPro, Ile-boroPro, Leu-boroPro or Glu-boroPro dissolved in normal saline or the saline vehicle alone were administered to BALB/c mice by oral gavage. Each mouse received a single administration of each amino boronic dipeptide or saline, and blood samples were withdrawn from mice 2 hours later.

Serum DPP-IV activity was determined by reaction of a 10 μl volume of serum with 0.1 mM Ala-Pro-AFC (Enzyme System Products, Dublin, Calif.) in a 100 μl volume of 50 mM HEPES/Na buffer pH 7.6, 140 mM NaCl. Assays were incubated for 30 min, stopped with 1-M sodium acetate, and fluorometric measurements were made as described in Example 1. Serum DPP-IV activity was expressed as fluorescent units (FU).

Serum concentration of mouse KC/CXCL1 was determined by Quantikine enzyme-linked immunosorbent assay (ELISA; R&D Systems, Minneapolis, Minn.) according to the manufacturer's instructions. ELISA was performed in duplicate for each sample.

Results

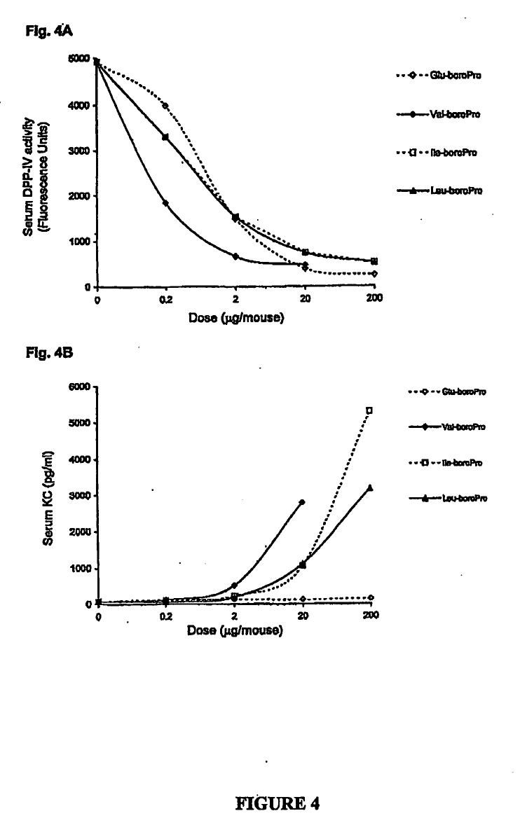

FIG. 4A illustrates typical in vivo dose responses for the inhibition of DPP-IV activity in the serum of BALB/c mice 2 hours following a single oral administration of Val-boroPro, Ile-boroPro, Leu-boroPro or Glu-boroPro. FIG. 4B illustrates the ability of Val-boroPro, Ile-boroPro and Leu-boroPro to stimulate increased serum levels of KC/CXCL1 in a dose-dependent manner. In marked contrast, Glu-boroPro failed to affect serum levels of KC/CXCL1 at any of the doses tested. The data of FIGS. 4A and 4B were obtained from the same serum samples collected from the mice after administration of the amino boronic dipeptides, thereby clearly demonstrating that the 20 and 200 μg doses of Glu-boroPro that maximally inhibited serum DPP-IV activity did not elicit a serum KC/CXCL1 response.

### Example 5

This example illustrates that among the amino boronic dipeptides shown to be potent inhibitors of DPP-IV in vitro, as indicated by IC₅₀ values in the low- to sub-nanomolar range (see Example 1), Glu-boroPro is distinguished by an ID₅₀ of 95 μg/kg and the lowest toxicity in Lewis rats [maximal tolerated dose (MTD) of 15 mg/kg administered as a single dose].

Materials and Methods

Serum DPP-IV inhibition and observation of acute toxicity of amino boronic dipeptides in Lewis rats. Groups of 2-3 rats were administered single escalating doses of Val-boroPro, Ile-boroPro, Met-boroPro, Leu-boroPro, Thr-boroPro, Gln-boroPro, Ala-boroPro, Lys-boroPro, Pro-boroPro, Arg-boroPro, Ser-boroPro or Glu-boroPro. Doses were initially increased in steps of 10 or 20 μg/kg in order to span a dose range of 10 to 200 μg/kg and in steps of 50 to 200 μg/kg for a higher dose range of 200-2000 μg/kg. Utilizing the DPP-IV assay described in example 2, serum DPP-IV activity was determined after 2 hours in rats administered Glu-boroPro and Val-boroPro. The health of the rats was monitored by visual inspection for a period of 5 days, thereby allowing the maximal tolerated dose (MTD) to be recorded for each amino boronic dipeptide as the dose level immediately beneath the dose that caused the rats to become moribund. After observations were completed or at the onset of a moribund state, humane euthanasia was performed by asphyxiation in 100% CO₂.

Results

The MTD obtained in acute toxicity studies in Lewis rats (Table 5) illustrate a range from 20 μg/kg for Val-boroPro to 15 mg/kg for Glu-boroPro. Interestingly, the dose responses for serum DPP-IV inhibition indicated ID₅₀ values of 9 μg/kg for Val-boroPro and 95 μg/kg for Glu-boroPro, following a single oral administration. Consequently, Glu-boroPro was only ˜10-fold less potent than Val-boroPro as an inhibitor of serum DPP-IV and yet was 750-fold less toxic.

TABLE 5
Maximum tolerated doses after acute (single dose)
administration of amino boronic dipeptides to Lewis rats

| Compound | MTD¹ (μg/kg) | ID₅₀² (μg/kg) |
|---|---|---|
| Val-boroPro | 20 | 9 |
| Ile-boroPro | 120 | NT³ |
| Met-boroPro | 160 | NT |
| Leu-boroPro | 200 | NT |
| Thr-boroPro | 800 | NT |
| Gln-boroPro | ≧800 | NT |
| Ala-boroPro | ≧2,000 | NT |
| Lys-boroPro | ≧2,000 | NT |
| Pro-boroPro | ≧2,000 | NT |
| Arg-boroPro | ≧2,000 | NT |
| Ser-boroPro | 4,000 | NT |
| Glu-boroPro | 15,000 | 95 |

¹Maximum tolerated dose
²Inhibitory dose 50%: i.e. dose causing a 50% reduction in serum DPP-IV activity from baseline in untreated animals
³Not tested

### Example 6

This example illustrates that mammalian cells are relatively impermeable to Glu-boroPro compared to another potent dipeptidyl peptidase inhibitor, Val-boroPro.

Materials and Methods

Intracellular expression of mvc-tagged dieptidyl peptidase-8 (DPP-8) in 293T cells. DPP-8 cDNA was amplified from cDNA prepared from RNA isolated by standard methods (as described in Example 1). The cDNA was prepared from 293T cells, but can be amplified from most cell types since DPP-8 is widely expressed (18). cDNA was cloned into a plasmid for preparation of 400 μg amounts for transfection experiments. Expression of the myc-tagged DPP-8 was achieved by transfection of the DPP-8-myc fusion plasmid into 293 T cells mediated by Lipofectamine 2000 transfection reagent as described in Example 1.

Post-extraction inhibition ofDPP-8 by amino boronic dipeptides. 293 T cells transfected with myc-DPP-8 were extracted with 1% Triton-X and 150 μl of extract incubated at room temperature with either Glu-boroPro or Val-boroPro at a concentration of 5.3 μM or without additions. After 15 minutes, 0.6 μg of anti-myc monoclonal antibody (mAb 9E10, Becton-Dickinson) was added and the mixture incubated for 3 hours on ice. Each reaction mixture was then split into 3 aliquots of 48 μl and each aliquot mixed with of protein G coupled beads (Sigma Chemical Co., St. Louis, Mo.) in 600 μl Triton lysis buffer and incubated for 1 hour at 4° C. The beads were washed twice in Triton lysis buffer and twice in assay buffer (140 mM NaCl, 50 mM HEPES pH 8.1), warmed to room temperature, mixed with 500 μM Ala-Pro-AFC in assay buffer and incubated for 4 min. The enzymatic reactions were stopped by addition of 1 M sodium acetate and measured fluorometrically as described in Example 1.

Pre-extraction inhibition of intracellular DPP-8 by amino boronic dipeptides. Viable 293 T cells transfected with myc-DPP-8 plasmid approximately 48 hours previously were released by trypsin treatment, spun down and resuspended in the same medium (Freestyle 293 Expression medium (InVitrogen Corporation) containing 5% Fetal Calf serum (HyClone)). The cell suspension was incubated approximately 35 minutes in a non-tissue culture treated petri dish at 37° C./ 5% CO₂ to allow recovery before centrifugation and resuspension in the same medium at 5×10⁶ cells per ml. Aliquots (150 microlitre) were incubated with either Glu-boroPro or Val-boroPro at a concentration of 10⁻⁴ M or without additions for 30 minutes at 37° C. The cells were then chilled on ice, washed 3 times to remove the inhibitors, and extracted with 0.8 ml 1% Triton-X lysis buffer as above. Myc-DPP-8 was immunoprecipitated and dipeptidyl peptidase activity assayed fluorometrically with Ala-Pro-AFC substrate as described above for the post-extraction protocol; but instead of stopping the reactions with 1 M sodium acetate, fluorescence was monitored continuously in the fluorometer for 15 minutes after the addition of substrate.

Results

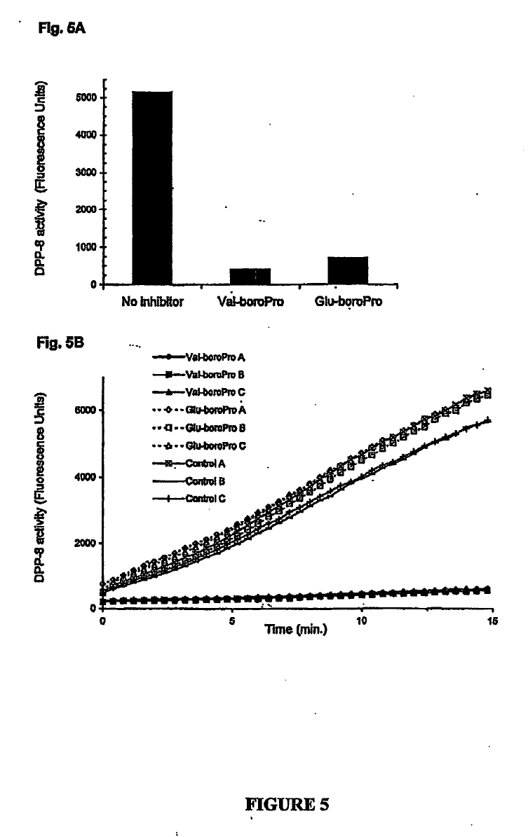

FIG. 5A illustrates the ability of 5.3-μM concentrations of both Val-boroPro and Glu-boroPro to inhibit the enzymatic activity of DPP-8 after extraction from myc-DPP-8 transfected 293 T cells. It should be noted that after incubation of cellular extracts with the amino boronic dipeptides, DPP-8 enzymatic activity remained inhibited after immunoprecipitation with anti-myc mAb. The relative stability of the complexes of DPP-8 and the amino boronic dipeptides demonstrated that intracellular DPP-8 could serve as an indicator of cell permeability to Val-boroPro and Glu-boroPro in the pre-extraction protocol. Utilizing this approach, in which intact, myc-DPP-8 transfected 293 T cells were incubated with the compounds before myc-DPP-8 was extracted, immunoprecipitated and assayed fluorometrically, it was found that 293 T cells were differentially permeable to Val-boroPro and Glu-boroPro. FIG. 5B illustrates that in triplicate samples (A, B and C) of myc-DPP-8 transfected 293 T cells incubated with 10⁻⁴ M concentrations of Val-boroPro or Glu-boroPro, only Val-boroPro appeared to enter the cells and inhibit intracellular DPP-8 activity.

### Example 7

This example illustrates that oral administration of Glu-boroPro to ob/ob mice 15 minutes prior to challenge by oral administration of glucose reduced the subsequent glucose excursion as indicated by determination of blood glucose levels.

Materials and Methods

Animals. Male, 10-week old ob/ob mice (background: C57BLKS/J) were obtained from Charles River Laboratories (USA) and kept in a 12/12 hour light-dark cycle with controlled temperature conditions (22-24° C). From time of arrival and throughout the experiment, mice were provided with standard rodent food (Altromin standard #1324 chow; C. Petersen, Ringsted, Denmark) and water ad libitum except were stated below.

Protocol for mouse oral glucose tolerance test (OGTT). The day of oral-glucose challenge was defined as day 0. On day −4, the mice were randomized (n=9 per group) to participate in one of the following drug-treatment groups: Groupl, vehicle (0.9% saline); Group 2, Glu-boroPro (1.0 μmol/kg). Agents were administered by oral gavage. Mice were restricted to a diet of 50% of their individual calculated food intake from day −1 onwards. On day 0, blood glucose was measured at t_{15 min.} immediately followed by drug administration. At time point 0, glucose was administered by oral gavage (1g/kg), and blood glucose was measured at time points 0, 30, 60, 120 and 240 minutes. Means±SE were calculated from the data of individual mice in drug-treatment groups 1 and 2. Statistical evaluation of the data was performed by one-way analysis of variance (ANOVA).

Results

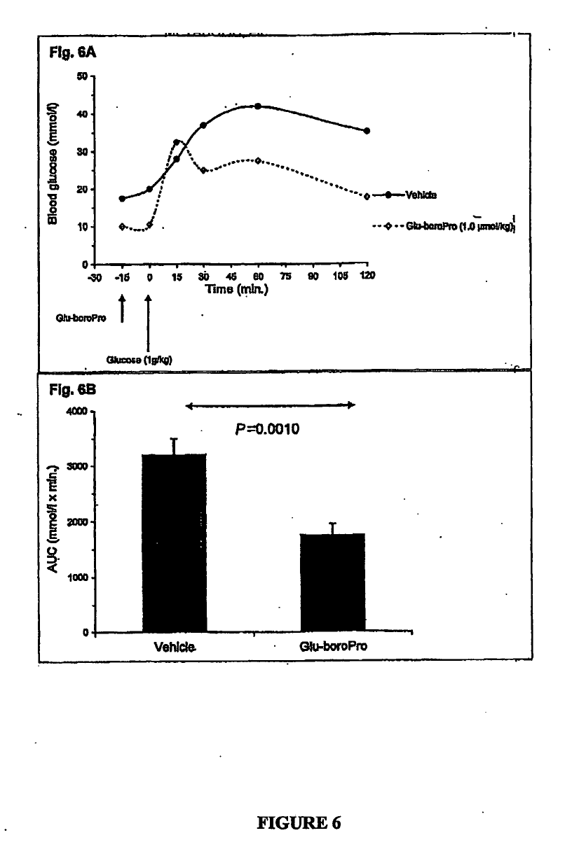

FIG. 6A illustrates the kinetic comparison of blood-glucose level between mice administered vehicle versus Glu-boroPro 15 minutes prior to oral glucose challenge. The glucose excursion post challenge was reduced by the Glu-boroPro treatment. Calculation of the area under the curves in FIG. 6B indicated that the anti-glycemic effect of the single 1.0 μmol/kg dose of Glu-boroPro was significant (P=0.0010).

### Example 8

This example illustrates that oral administration of Glu-boroPro to Zucker rats 15 minutes prior to challenge by oral administration of glucose reduced the subsequent glucose excursion, increased insulin and GLP-1 responses, and inhibited blood plasma DPP-IV activity, as indicated by the appropriate assays of blood levels.

Materials and Methods

Animals. 6-week old male Zucker fa/fa rats were obtained from Charles River Laboratories, USA) and housed in a 12/12 hour light-dark cycle with controlled temperature conditions (22-24° C.). From time of arrival and throughout the experiment, rats were provided with standard rodent food (Altromin standard #1324 chow; C. Petersen, Ringsted, Denmark) and water ad libitum except were stated below.

Protocol for rat oral glucose tolerance test (OG7T). The day of experimental oral-glucose challenge was defined as day 0. On days −11 to −8, rats were fitted with intra-arterial catheters under light isoflurane anesthesia. On day −1, the rats were stratified according to a randomization OGTT performed on day −6. Rats were randomized (n=6 per group) to participate in one of the following drug-treatment groups: Group1, vehicle (0.9% saline); Group 2, Glu-boroPro (10.0 μmol/kg). From 12:00 a.m. (noon) on day −5, rats were offered only 50% of their individual 24-hour food intake. On day 0, drugs were administered by oral gavage at time point t-₁₅ min relative to time point 0 when glucose was administered by oral gavage (2 g/kg). Blood was sampled for analysis according to the following schedule, according to Table 6.

TABLE 6
Blood analysis scheme

| | Blood sample volume (ml) | | |
|---|---|---|---|
| | collected for assay of: | | |
| | Glucose and | | |
| Time point | insulin | DPP-IV | GLP-1 |

| | | | | |
|---|---|---|---|---|
| −15 | min. | 0.3 | | 0.5 |
| −5 | min. | 0.3 | 0.2 | |
| 0 | | 0.3 | 0.2 | 0.5 |
| 5 | min. | 0.3 | 0.2 | 0.5 |
| 10 | min. | 0.3 | | 0.5 |
| 15 | min. | 0.3 | | 0.5 |
| 20 | min. | 0.3 | 0.2 | |
| 30 | min. | 0.3 | | |
| 45 | min. | 0.3 | | |
| 60 | min. | 0.3 | | |
| 90 | min. | 0.3 | 0.2 | |
| 120 | min. | 0.3 | 0.2 | |
| 240 | min. | 0.3 | 0.2 | |
| 24 | hours | 0.3 | 0.2 | |
| 48 | hours | 0.3 | 0.2 | |

Serum DPP-IV activity was assayed fluorometrically as in Example 2, except that the substrate Gly-boroPro was substituted for Ala-boroPro as described elsewhere (11). Blood-plasma glucose was assayed with an automated analyzer (Roche Diagnostics). Active GLP-1 levels were determined in duplicate from each blood sample by ELISA (Linco Research, St. Charles, Mo.) and, similarly, P-insulin was measured by ELISA (Diamyd, Sweden). Means±SE were calculated from the data of individual rats in drug treatment groups 1 and 2. Statistical evaluation of the data was performed by one-way analysis of variance (ANOVA).

Results

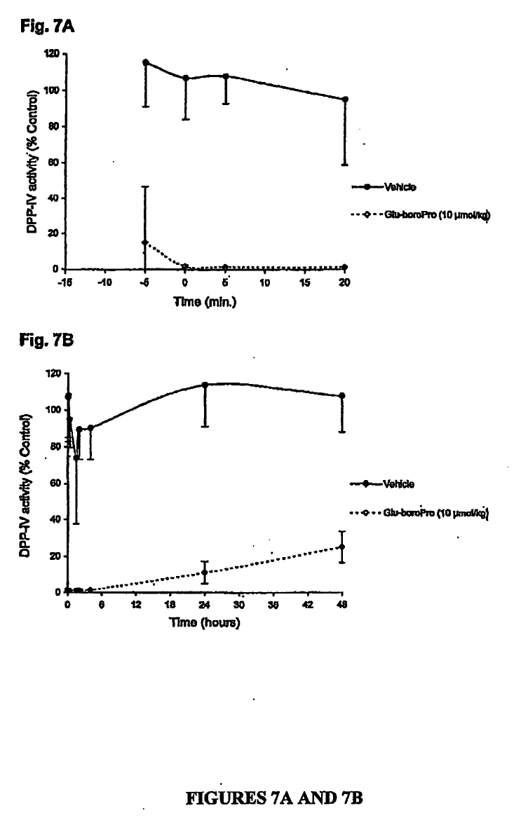

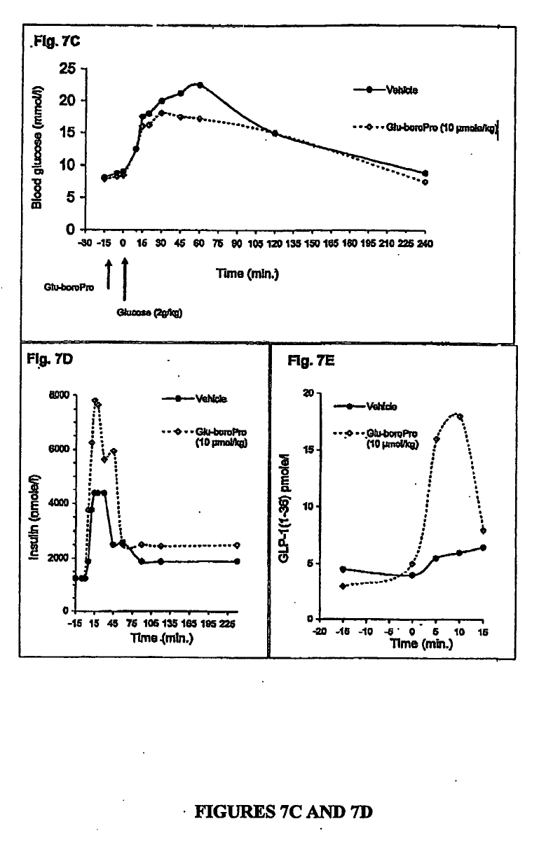

FIG. 7 illustrates the kinetics of serum DPP-IV inhibition following a single oral administration of a 10 μmol/kg dose of Glu-boroPro to Zucker rats at t₁₅ₘᵢₙ, relative to oral glucose challenge at time point 0. FIG. 7A illustrates DPP-IV activity at early time points: −5, 0, 5, and 20 minutes and FIG. 7B illustrates the complete kinetics up to the final measurement of DPP-IV activity at 48 hours. Marked inhibition of plasma DPP-IV activity was observed at t₅ₘᵢₙ and maximal inhibition was achieved by t₀ (FIG. 7A). Maximal inhibition of DPP-IV activity persisted until at least t₄ₕₒᵤᵣₛ (FIG. 7B). Plasma DPP-IV activity recovered to reach levels of 11% and 25% of control values at t₂₄ₕₒᵤᵣₛ and t₄₈ₕₒᵤᵣₛ, respectively.

FIG. 7C illustrates that blood glucose excursion was reduced by Glu-boroPro administration. DPP-IV inhibitors reduce blood glucose excursions in the OGTT by preventing the proteolytic degradation of GLP-1, which in turn results in an increased incretin effect on insulin secretion by pancreatic β-cells (4, 11-13, 19). In agreement with this mechanism of action, Glu-boroPro administration increased the blood plasma levels of both insulin and GLP-1 following oral glucose challenge in Zucker rats (FIGS. 7D and E). The inhibition of blood plasma DPP-IV activity observed after oral administration of the single dose of Glu-boroPro was clearly sufficiently rapid (FIG. 7A) to account for the increased levels of active GLP-1 (FIG. 7E).

### Example 9

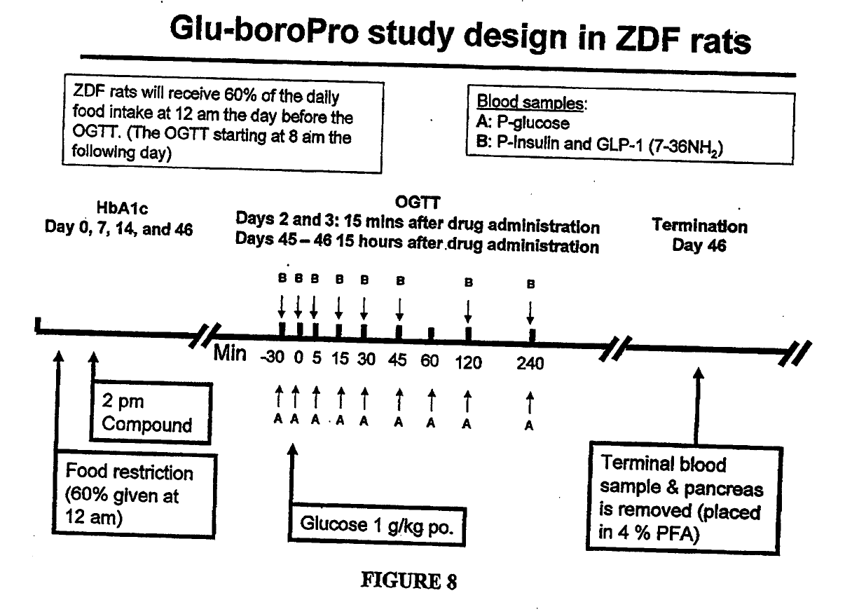

The design of the 6 week long study is depicted in FIG. 8. 60 male Zucker Diabetic Fatty (ZDF) rats (5 weeks of age) were obtained from Charles River Labs. The animals are housed singly and allowed to acclimatize for 1 week before the initiation of the study. 24-hour food intake and body weight was measured gravimetrically twice weekly from the start of housing. HbA₁C levels were measured at Day 0, 7, 14, 28 and 46. OGTT was administered on Day 2-3, 15 minutes after the administration of the drugs. OGTT was administered again at the end of the study but in this case, drugs were given 15 hours prior to the OGTT. The 24-hour glucose profile was monitored at day 14-15 of the study.

Results

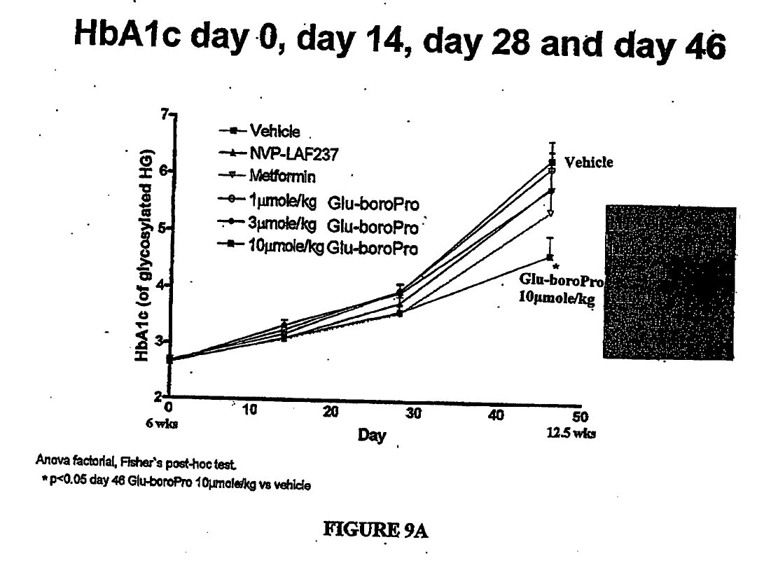

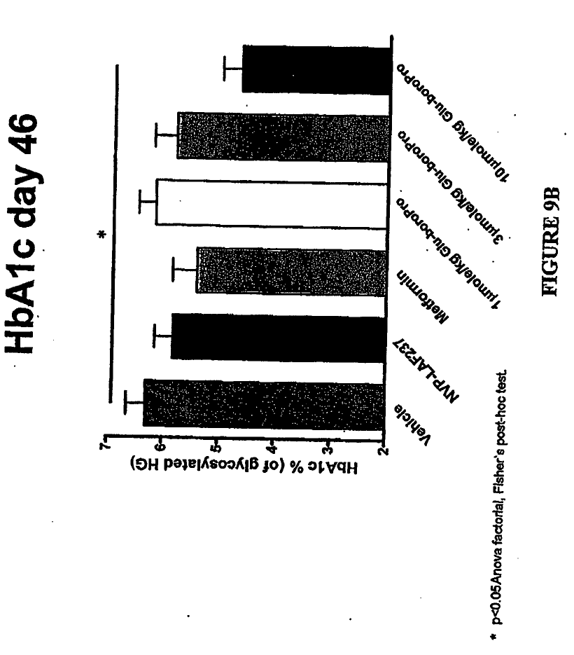

FIG. 9A shows the HbA₁C levels monitored at Day 0 (initiation of the study), Day 7, Day 14, Day 30 and Day 46. There was an increase in HbA₁C level over time in all groups due to the severity and worsening of diabetes in these animals. The decline in the rate of increase in HbA₁C was most prominent in the Glu-boroPro 10 micromoles/kg group.

FIG. 9B shows that at Day 46, the only group to achieve a significant reduction in HbA₁C level compared to the vehicle treated group was the Glu-boroPro 10 micromole/kg dose group.

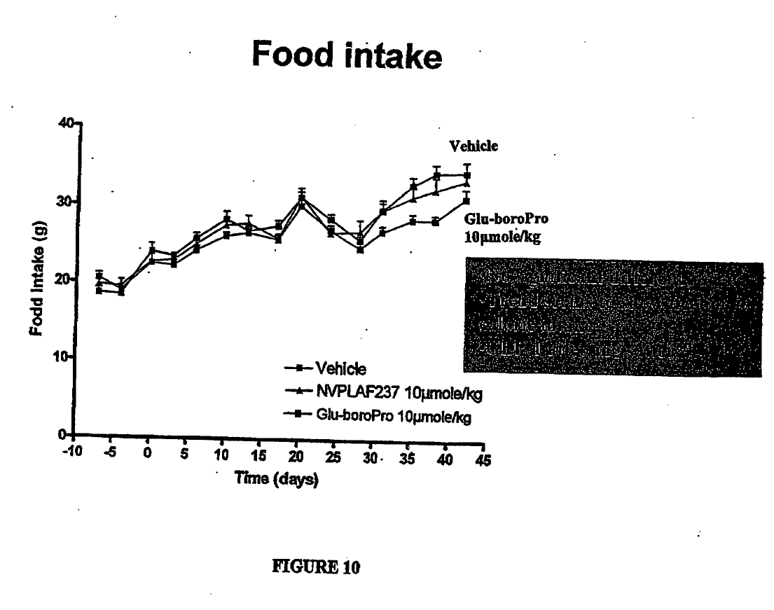

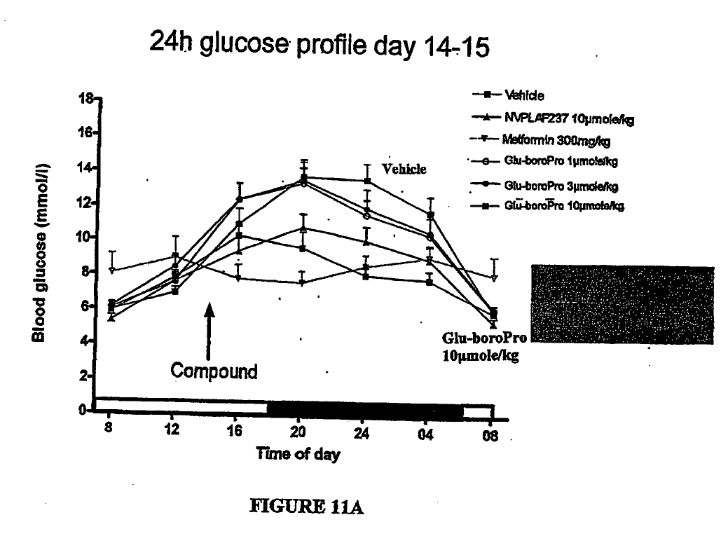

FIG. 10 shows an apparent slight decrease in food intake in the Glu-boroPro 10 micromole/kg group over time. However, the difference with the vehicle treated group did not reach significance.

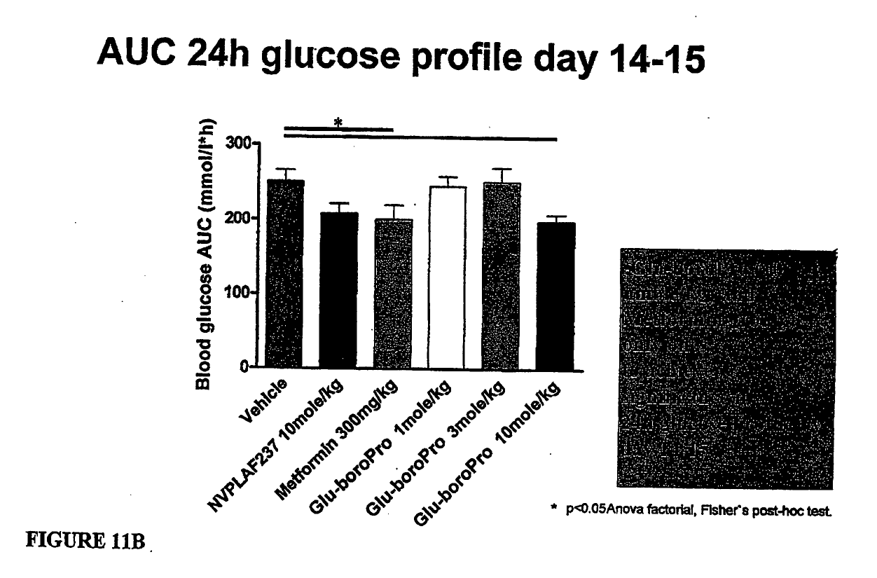

FIG. 11A depicts the 24-hour glucose profile conducted in day 14-15 of the study. The glucose profile in the Glu-boroPro 10 micromole/kg group was improved compared to the metformin and the NVP-LAF237 groups. This is a double baseline plot and, therefore, the last and the first time-points have the same values.

The area under the curve (AUC) measurement for the 24 hour glucose profile (FIG. 11B) shows that the Glu-boroPro (10 μmole/kg) and Metformin groups were the only ones to achieve significance.

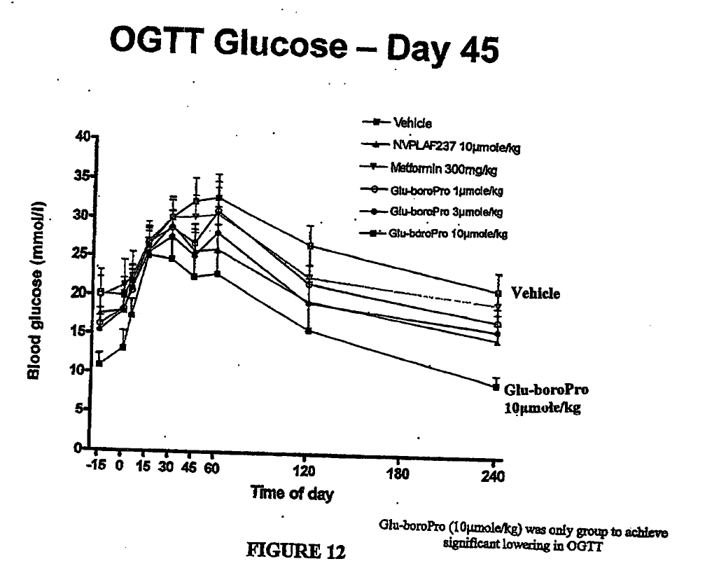

The OGTT given 15 hours after drug administration (FIG. 12) demonstrated that the Glu-boroPro 10 micromole/kg group achieved significant reduction in glucose levels. The other groups failed to reach significance in their reduction compared to the vehicle treated group.

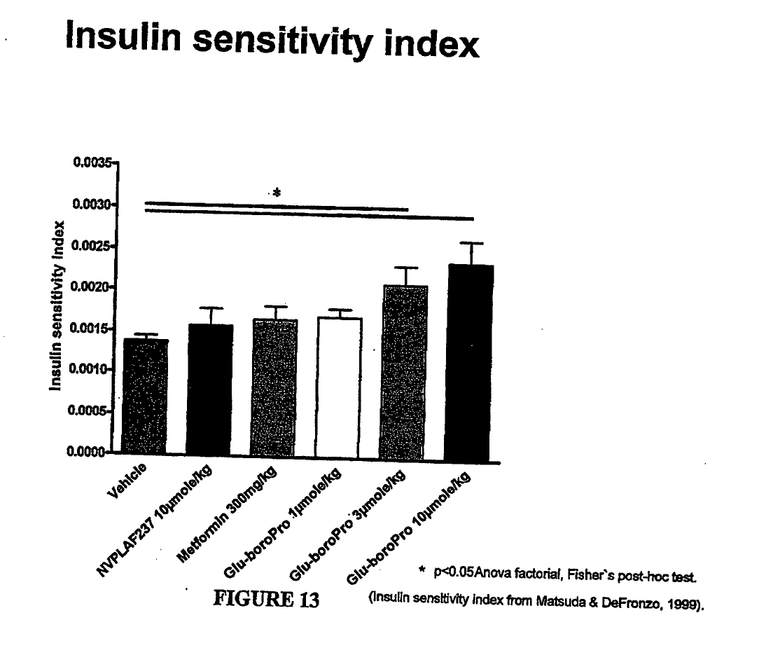

The insulin sensitivity index was calculated at the termination of the study according to the method described by Matsuda and DeFronzo (Diabetes Care. 1999 Sep.;22(9):1462-70). FIG. 13 shows that Glu-boroPro 3 micromole/kg and 10 micromole/kg were the only groups to achieve a significant improvement. There was a dose response relationship for the Glu-boroPro group and the improvements in the insulin sensitivity index.

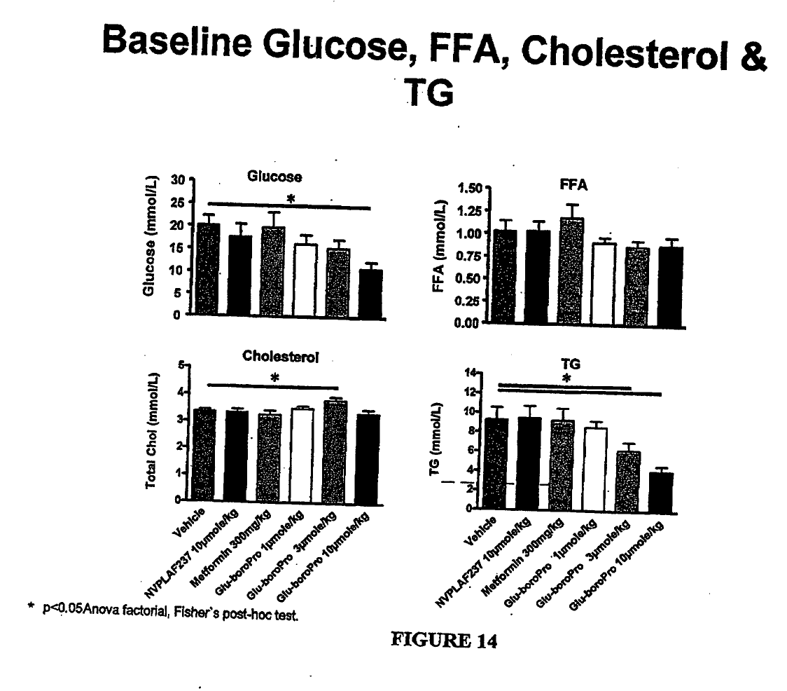

FIG. 14 shows that there was a significant improvement in triglyceride (TG) lowering after 46 days of treatment in the Glu-boroPro 3 micromole/kg and 10 micromole/kg groups. There was also a trend towards free fatty acids (FFA) reduction but this did not reach significance compared to the vehicle treated group within this time point. Cholesterol measurement did not distinguish between HDL and LDL.

### Example 10

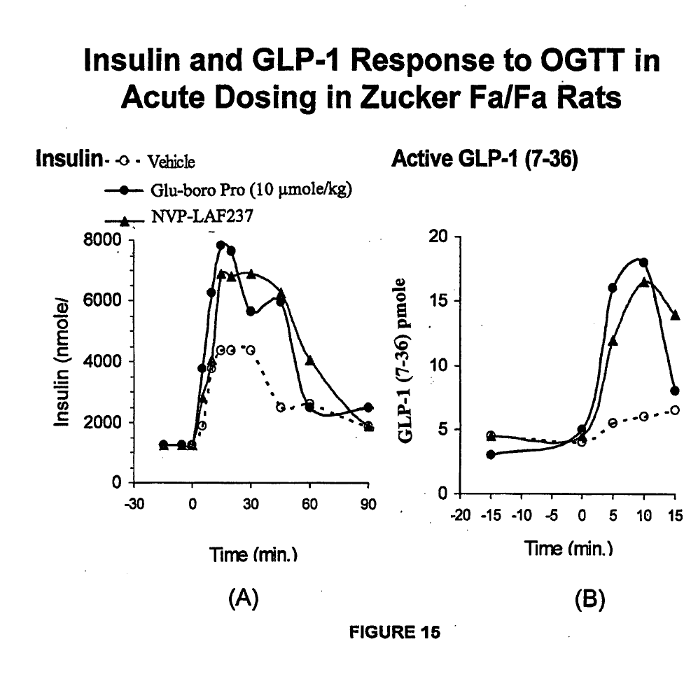

Oral administration of a single dose of Glu-boroPro to Zucker Fa/Fa rats, 15 minutes prior to an oral glucose challenge, resulted in an improved insulin response and increased levels of the un-degraded, active form of GLP-1. The data shown in FIG. 15 are mean values from 6 rats per treatment. The effect of PT-630 was equivalent to that achieved with an optimal dose of the cyano-pyrrolidine (CP) inhibitor of DPP-IV (NVP-LAF237).

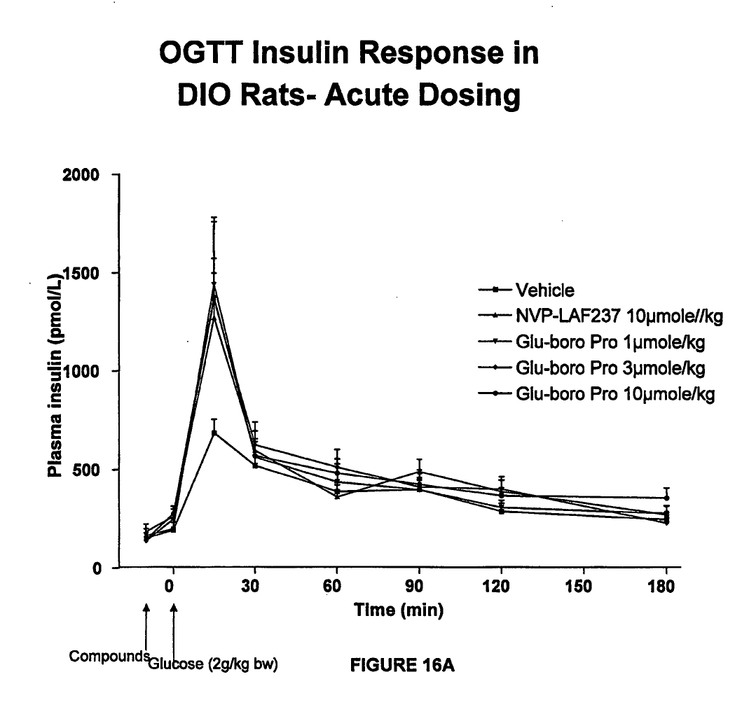

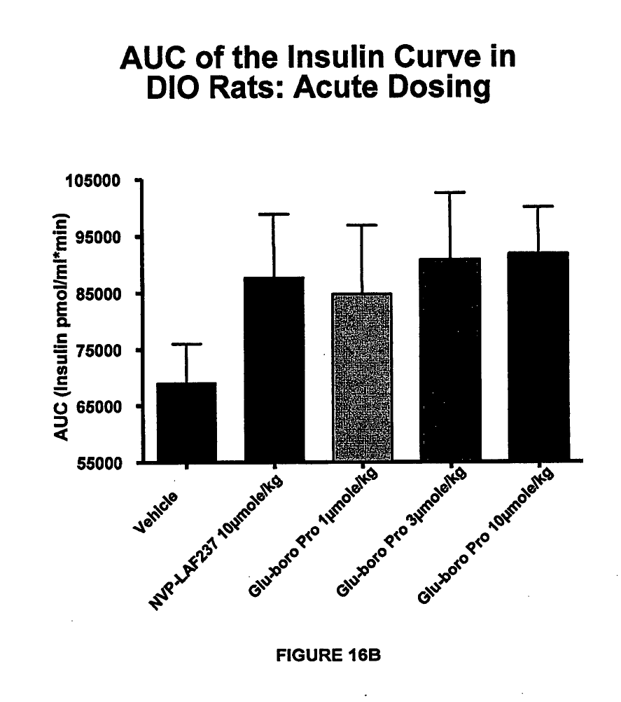

Oral administration of a single dose of Glu-boroPro to DIO rats, 15 minutes prior to an oral glucose challenge, resulted in an improved insulin response. The data shown in FIG. 16A are mean values from 10 rats per treatment. The effect of PT-630 was equivalent to that achieved with an optimal dose of the cyano-pyrrolidine (CP) inhibitor of DPP-IV (NVP-LAF237).

Oral administration of a single dose of PT-630 to DIO rats, 15 minutes prior to oral glucose challenge, resulted in an increased insulin secretion as calculated from AUC of the insulin secretion. The data shown in FIG. 16B are mean values from 10 rats per treatment. The effect of PT-630 was equivalent to that achieved with an optimal dose of the cyano-pyrrolidine (CP) inhibitor of DPP-IV (NVP-LAF237).

### Example 11

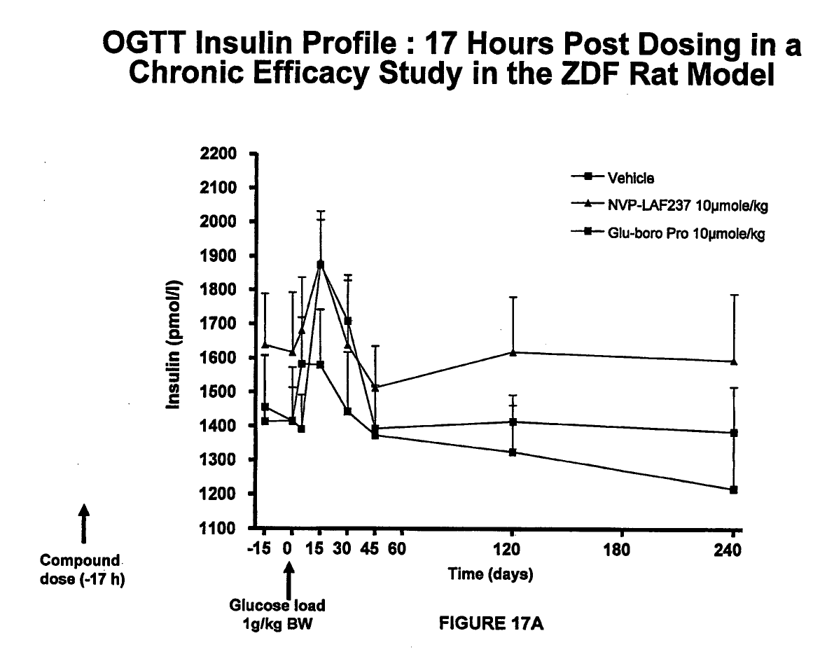

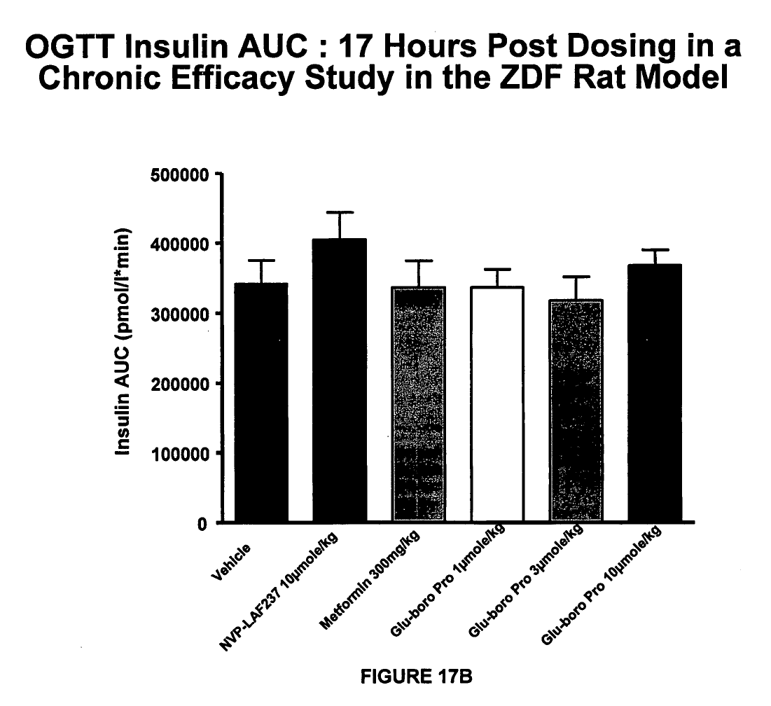

A 6-week long study was conducted which included 60 males (5 weeks of age) Zucker Diabetic Fatty (ZDF) rats obtained from Charles River Laboratories (Wilmington, Mass.). The animals were housed singly and allowed to acclimatize for 1 week before the initiation of the study. Animals were dosed once daily with NVP-LAF237 (10 μmole/kg), Glu-boroPro (10 μmole/kg) or vehicle for 6 weeks. OGTT was administered at the end of the study and drugs were given 15 hours prior to the OGTT. The insulin response during the OGTT is shown in FIG. 17A. The area under the insulin curve is shown in bar graphs in FIG. 17B. The data are mean values from 10 rats per treatment. The data are mean values from 10 rats per treatment.

### REFERENCES

1. Taylor, S. I. Deconstructing type 2 diabetes. Cell, 97: 9-12, 1999.
2. UKPDS UK prospective diabetes study 33: intensive blood glucose control with sulphonylureas or insulin compared with conventional treatment and risk of complications with type 2 diabetes. Lancet, 352: 837-853, 1998.
3. Drucker, D. J. The glucagon-like peptides. Endocrinology, 142: 521-527, 2001.
4. Moller, D. E. New drug targets for type 2 diabetes and metabolic syndrome. Nature, 414: 821-827, 2001.
5. de Meester, I., Lambeir, A. M., Proost, P., and Scharpe, S. Dipeptidyl peptidase IV substrates. An update on in vitro peptide hydrolysis by human DPP-IV. Adv. Exp. Med. Biol., 524: 3-17, 2003.
6. de Meester, I., Korom, S., Van Damme, J., and Scharpe, S. CD26, let it cut or cut it down. Immunol. Today, 20: 367-375, 1999.
7. Drucker, D. J. Biological actions and therapeutic potential of the glucagon-like peptides. Gastroenterology, 122: 531-544, 2002.
8. Marguet, D., Baggio, L., Kobayashi, T., Bernard, A. -M., Pierres, M., Nielsen, P. F., Ribel, U., Watanabe, T., Drucker, D. J., and Wagtmann, N. Enhanced insulin secretion an improved glucose tolerance in mice lacking CD26. Proc. Natl. Acad. Sci. USA, 97: 6874-6879, 2000.
9. Baikan, B., Kwasnik, L., Miserendino, R., Holst, J. J., and Li, X. Inhibition of dipeptidyl peptidase IV with NVP-DPP728 increases plasma GLP-1 (7-36 amide) concentrations and improves oral glucose tolerance in obese Zucker rats. Diabetologia, 42: 1324-1331, 1999.
10. Ahren, B., Holst, J. J., Martensson, H., and Balkan, B. Improved glucose tolerance and insulin secretion by inhibition of dipeptidyl peptidase IV in mice. Eur. J. Pharmacol., 404: 239-245, 2000.
11. Villhauer, E. B., Brinkman, J. A., Naderi, G. B., Burkey, B. F., Dunning, B. E., Prasad, K., Mangold, B. L., Russell, M. E., and Hughes, T. E. 1-[[3-Hydroxy-1-adamantyl) amino]acetyl]-2-cyano-(S)-pyrrolidine: a potent, selective, and orally bioavailable dipeptidyl peptidase IV inhibitor with antihyperglycemic properties. J. Med. Chem., 46: 2774-2789, 2003.
12. Ahren, B. and al, e. Inhibition of dipeptidyl peptidase IV improves metabolic control over a 4-week study period in type 2 diabetes. Diabetes Care, 25: 869-875, 2002.
13. Demuth, H. -U. e. a. Single dose treatment of diabetic patients by the DP-IV inhibitor P32/98. Diabetes, 49(Suppl. 1): A102, 2000.
14. Conarello, S. L., Li, Z., Ronan, J., Roy, R. S., Zhu, L., Jiang, G., Liu, F., Woods, J., Zycband, E., Moller, D. E., Thomberry, N. A., and Zhang, B. B. Mice lacking dipeptidyl peptidase IV are protected against obesity and insulin resistance. Proc. Natl. Acad. Sci. USA, 100: 6825-6830, 2003.
15. Durinx, C., Lambeir, A. M., E., B., Falmagne, J. B., Berghmans, R., A., H., Scharpe, S., and de Meester, I. Molecular characterization of dipeptidyl peptidase activity in serum: soluble CD26/dipeptidyl peptidase IV is responsible for the release of dipeptides. Eur. J. Biochem., 267: 5608-5613, 2000.
16. Jones, B., Adams, S., Miller, G. T., Jesson, M. I., Watanabe, T., and Wallner, B. P. Hematopoietic stimulation by a dipeptidyl peptidase inhibitor reveals a novel regulatory mechanism and therapeutic treatment for blood cell deficiencies. Blood, 102: 1641-1648, 2003.
17. Adams, S., Miller, G. T., Jesson, M. I., Watanabe, T., Jones, B., and Wallner, B. P. PT-100, a small molecule dipeptidyl peptidase inhibitor, has potent anti-tumor effects and augments antibody-mediated cytotoxicity via a novel immune mechanism. Cancer Res., 64: 5471-5480, 2004.
18. Abbott, C. A., Yu, D. M. T., Woollatt, E., Sutherland, G. R., and McCaughan, G. W. Cloning, expression and chromosomal localization of a novel human dipeptidyl peptidase (DPP) IV homolog, DPP8. Eur. J. Biochem., 267: 6140-6150, 2000.
19. Balkan, B., Kwasnik, L., Miserendino, R., Holst, J. J., and Li, X. Inhibition of dipeptidyl peptidase IV with NVP-DPP728 increases plasma GLP-1 (7-36 amide) concentrations and improves oral glucose tolerance in obese Zucker rats. Diabetologia, 42: 1324-1331, 1999.

### EQUIVALENTS

The foregoing written specification is considered to be sufficient to enable one ordinarily skilled in the art to practice the invention. The present invention is not to be limited in scope by examples provided, since the examples are intended as mere illustrations of one or more aspects of the invention. Other finctionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

All references, patents and patent applications that are recited in this application are incorporated by reference herein in their entirety.

What is claimed is:

1. A method for treating a subject having or at risk of developing a glucose-associated condition comprising
administering to a subject in need thereof an agent comprising 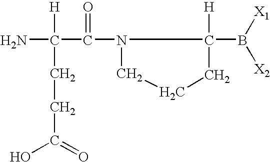
or a prodrug thereof in an effective amount to treat the subject wherein each X₁ and X₂ is, independently, a hydroxyl group or a group capable of being hydrolyzed to a hydroxyl group in aqueous solution at physiological pH.

2. (canceled)

3. The method of claim 1, wherein the agent is administered orally.

4-39. (canceled)

40. The method of claim 1, wherein the agent is a cyclic version of Glu-boroPro.

41. (canceled)

42. The method of claim 1, wherein the agent has a structure 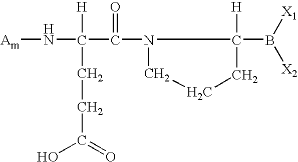
wherein A is any naturally or non-naturally occurring amino acid bonded in either an S- or an R-configuration; m is an integer from 0-100, such that when m is greater than one, each A in Aₘ may be a different amino acid residue from every other A in Aₘ; and each X₁ and X₂ is, independently, a hydroxyl group or a group capable of being hydrolyzed to a hydroxyl group in aqueous solution at physiological pH.

43. The method of claim 1, wherein the agent has a structure 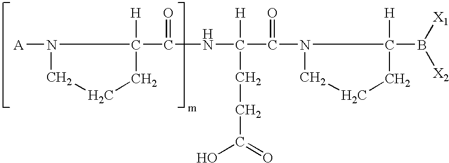
wherein A is any naturally or non-naturally occurring amino acid in an S- or an R-configuration; m is an integer from 0-100, provided that when m is greater than one, A in each repeating bracketed unit can be a different amino acid residue; and each X₁ and X₂ is, independently, a hydroxyl group or a group capable of being hydrolyzed to a hydroxyl group in aqueous solution at physiological pH.

44-48. (canceled)

49. A composition comprising
an agent comprising 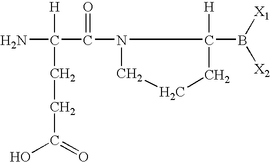
or a prodrug thereof and a second therapeutic agent, wherein each X₁ and X₂ is, independently, a hydroxyl group or a group capable of being hydrolyzed to a hydroxyl group in aqueous solution at physiological pH.

50-84. (canceled)

85. A pharmaceutical composition comprising
an agent comprising 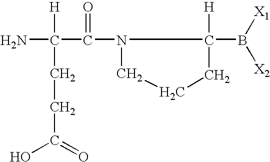
or a prodrug thereof in a pharmaceutically-acceptable carrier and in a unit dosage that is effective for reducing blood glucose in a subject wherein each X₁ and X₂ is, independently, a hydroxyl group or a group capable of being hydrolyzed to a hydroxyl group in aqueous solution at physiological pH.

86-103. (canceled)

104. A method for reducing blood glucose comprising
orally administering to a subject in need thereof prior to a glucose challenge Glu-boroPro having the structure 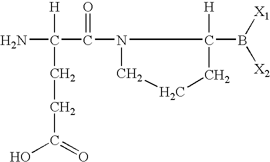
in an effective amount to reduce blood glucose level wherein each X₁ and X₂ is, independently, a hydroxyl group or a group capable of being hydrolyzed to a hydroxyl group in aqueous solution at physiological pH.

105-117. (canceled)

118. A method for treating a subject having or at risk of developing a dyslipidemia comprising
administering to a subject in need thereof an agent having a structure 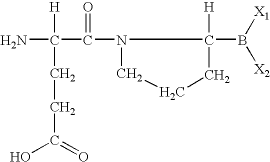
or a prodrug thereof in an effective amount to treat the subject wherein each X₁ and X₂ is, independently, a hydroxyl group or a group capable of being hydrolyzed to a hydroxyl group in aqueous solution at physiological pH.

119-146. (canceled)

147. A method for treating a subject having or at risk of developing metabolic syndrome comprising
administering to a subject in need thereof an agent having a structure 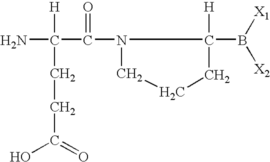
or a prodrug thereof in an effective amount to treat the subject wherein each X₁ and X₂ is, independently, a hydroxyl group or a group capable of being hydrolyzed to a hydroxyl group in aqueous solution at physiological pH.

148-163. (canceled)

164. A method for treating a subject having or at risk of developing a cardiovascular disorder comprising
administering to a subject in need thereof an agent having a structure 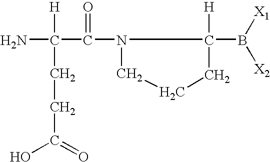
or a prodrug thereof in an effective amount to treat the subject wherein each X₁ and X₂ is, independently, a hydroxyl group or a group capable of being hydrolyzed to a hydroxyl group in aqueous solution at physiological pH.

165-178. (canceled)

179. A method for controlling body weight of a subject comprising
administering to a subject in need thereof an agent having a structure: 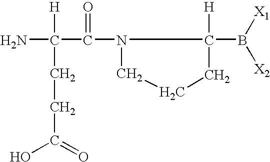
or a prodrug thereof in an effective amount to control body weight of the subject wherein each X₁ and X₂ is, independently, a hydroxyl group or a group capable of being hydrolyzed to a hydroxyl group in aqueous solution at physiological pH.

180-191. (canceled)

192. A method for increasing the insulin sensitivity index in a subject comprising
administering to a subject in need thereof an agent having a structure 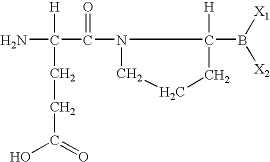
or a prodrug thereof in an effective amount to increase the insulin sensitivity in the subject wherein each X₁ and X₂ is, independently, a hydroxyl group or a group capable of being hydrolyzed to a hydroxyl group in aqueous solution at physiological pH.

193-207. (canceled)

208. A method for lowering free fatty acid levels in a subject comprising
administering to a subject in need thereof an agent having a structure 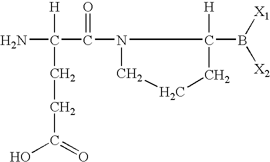
or a prodrug thereof in an effective amount to lower free fatty acid levels in the subject wherein each X₁ and X₂ is, independently, a hydroxyl group or a group capable of being hydrolyzed to a hydroxyl group in aqueous solution at physiological pH.

209-223. (canceled)

224. A method for lowering HbA₁C level in a subject comprising administering to a subject in need thereof an agent having a structure 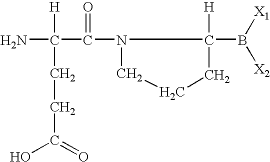
or a prodrug thereof in an effective amount to lower the HbA₁C level in the subject wherein each X₁ and X₂ is, independently, a hydroxyl group or a group capable of being hydrolyzed to a hydroxyl group in aqueous solution at physiological pH.

225-236. (canceled)

237. A method for treating a subject having or at risk of developing an elevated triglyceride level comprising
administering to a subject in need thereof an agent having a structure 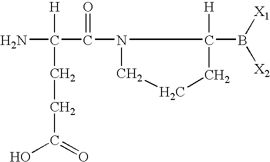
or a prodrug thereof in an effective amount to lower triglyceride level in the subject wherein each X₁ and X₂ is, independently, a hydroxyl group or a group capable of being hydrolyzed to a hydroxyl group in aqueous solution at physiological pH.

238-243. (canceled)

244. A method for lowering an elevated C-reactive protein (CRP) level in a subject comprising
administering to a subject in need thereof an agent having a structure 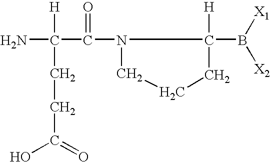
or a prodrug thereof in an effective amount to lower CRP level in the subject wherein each X₁ and X₂ is, independently, a hydroxyl group or a group capable of being hydrolyzed to a hydroxyl group in aqueous solution at physiological pH.

245-250. (canceled)

251. A method for treating a subject having or at risk of developing inflammation or an inflammatory disorder comprising
administering to a subject in need thereof an agent having a structure 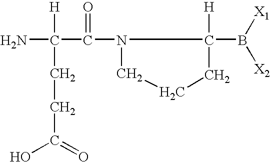
or a prodrug thereof in an effective amount to treat the subject wherein each X₁ and X₂ is, independently, a hydroxyl group or a group capable of being hydrolyzed to a hydroxyl group in aqueous solution at physiological pH.

252-277. (canceled)

278. A method for stimulating insulin release from a pancreatic cell comprising
contacting a pancreatic cell with a Glu-boroPro containing compound in an effective amount to stimulate insulin release from the pancreatic cell and increase insulin level.

279-297. (canceled)

298. A method for increasing adiponectin level in a subject comprising
administering to a subject in need thereof an agent having a structure 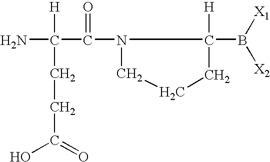
or a prodrug thereof in an effective amount to increase adiponectin level in the subject wherein each X₁ and X₂ is, independently, a hydroxyl group or a group capable of being hydrolyzed to a hydroxyl group in aqueous solution at physiological pH.

299-304. (canceled)


SEQUENCE LISTING (7 sequences)

SEQ ID NO: 1 - protein, 51 aa, Homo sapiens
MKTPWKVLLGLLGAAALVTIITVPVVLLNKGTDDATADSRKTYTLTDYLKN

SEQ ID NO: 2 - protein, 13 aa, Homo sapiens
SRKTYTLTDYLKN

SEQ ID NO: 3 - protein, 12 aa, Homo sapiens
RKTYTLTDYLKN

SEQ ID NO: 4 - DNA, 49 bp, Artificial sequence
  Synthetic oligonucleotide
gtagtcggcccagccggccagtcgcaaaacttacactctaactgattac

SEQ ID NO: 5 - DNA, 47 bp, Artificial sequence
  Synthetic oligonucleotide
gtcggagcggccgcctaaggtaaagagaaacattgttttatgaagtg

SEQ ID NO: 6 - protein, 6 aa, Artificial sequence
  Synthetic peptide
DAAQPR

SEQ ID NO: 7 - protein, 6 aa, Artificial sequence
  Synthetic peptide
DAAQPA